SEQUENCE LISTING (13 sequences)

SEQ ID NO: 1 - DNA, 24 bp, Artificial Sequence
  Primer
cgtccaggagcgcaccatcttctt

SEQ ID NO: 2 - DNA, 24 bp, Artificial Sequence
  Primer
atcgcgcttctcgttggggtcttt

SEQ ID NO: 3 - DNA, 30 bp, Artificial Sequence
  Primer
aggactgggtggcttccaactcccagacac

SEQ ID NO: 4 - DNA, 30 bp, Artificial Sequence
  Primer
agcttctcattgctgcgcgccaggttcagg

SEQ ID NO: 5 - DNA, 22118 bp, Mus musculus
gaattcccctatccctaatccagattggtggaataacttggtatagatgtttgtgcattaaaaaccctgtaggatcttcactctaggtcactgttcagcactggaacctgaattgtggccctgagtgataggtcctgggacatatgcagttctgcacagacagacagacagacagacagacagacagacagacagacgttacaaacaaacacgttgagccgtgtgccaacacacacacaaacaccactctggccataattattgaggacgttgatttattattctgtgtttgtgagtctgtctgtctgtctgtctgtctgtctgtctgtctatcaaaccaaaagaaaccaaacaattatgcctgcctgcctgcctgcctgcctacacagagaaatgatttcttcaatcaatctaaaacgacctcctaagtttgccttttttctctttctttatctttttcttttttcttttcttcttccttccttccttccttccttccttccttcctttctttctttctttctttctttcttactttctttctttccttcttacatttattcttttcatacatagtttcttagtgtaagcatccctgactgtcttgaagacactttgtaggcctcaatcctgtaagagccttcctctgcttttcaaatgctggcatgaatgttgtacctcactatgaccagcttagtcttcaagtctgagttactggaaaggagttccaagaagactggttatatttttcatttattattgcattttaattaaaatttaatttcaccaaaagaatttagactgaccaattcagagtctgccgtttaaaagcataaggaaaaagtaggagaaaaacgtgaggctgtctgtggatggtcgaggctgctttagggagcctcgtcaccattctgcacttgcaaaccgggccactagaacccggtgaagggagaaaccaaagcgacctggaaacaataggtcacatgaaggccagccacctccatcttgttgtgcgggagttcagttagcagacaagatggctgccatgcacatgttgtctttcagcttggtgaggtcaaagtacaaccgagtcacagaacaaggaagtatacacagtgagttccaggtcagccagagtttacacagagaaaccacatcttgaaaaaaacaaaaaaataaattaaataaatataatttaaaaatttaaaaatagccgggagtgatggcgcatgtctttaatcccagctctcttcaggcagagatgggaggatttctgagtttgaggccagcctggtctgcaaagtgagttccaggacagtcagggctatacagagaaaccctgtcttgaaaactaaactaaattaaactaaactaaactaaaaaaatataaaataaaaattttaaagaattttaaaaaactacagaaatcaaacataagcccacgagatggcaagtaactgcaatcatagcagaaatattatacacacacacacacacagactctgtcataaaatccaatgtgccttcatgatgatcaaatttcgatagtcagtaatactagaagaatcatatgtctgaaaataaaagccagaaccttttctgcttttgttttcttttgccccaagatagggtttctctcagtgtatccctggcatccctgcctggaacttcctttgtaggtttggtagcctcaaactcagagaggtcctctctgcctgcctgcctgcctgcctgcctgcctgcctgcctgcctgcctgcctcacttcttctgccacccacacaaccgagtcgaacctaggatctttatttctttctctttctctcttctttctttctttctttctttctttctttctttctttctttctttctttcttattcaattagttttcaatgtaagtgtgtgtttgtgctctatctgctgcctataggcctgcttgccaggagagggcaacagaacctaggagaaaccaccatgcagctcctgagaataagtgaaaaaacaacaaaaaaaggaaattctaatcacatagaatgtagatatatgccgaggctgtcagagtgctttttaaggcttagtgtaagtaatgaaaattgttgtgtgtcttttatccaaacacagaagagaggtggctcggcctgcatgtctgttgtctgcatgtagaccaggctggccttgaacacattaatctgtctgcctctgcttccctaatgctgcgattaaaggcatgtgccaccactgcccggactgatttcttcttttttttttttttggaaaatacctttctttctttttctctctctctttcttccttccttcctttctttctattctttttttctttcttttttctttttttttttttttttaaaatttgcctaaggttaaaggtgtgctccacaattgcctcagctctgctctaattctctttaaaaaaaaacaaacaaaaaaaaaaccaaaacagtatgtatgtatgtatatttagaagaaatactaatccattaataactcttttttcctaaaattcatgtcattcttgttccacaaagtgagttccaggacttaccagagaaaccctgtgttcaaatttctgtgttcaaggtcaccctggcttacaaagtgagttccaagtccgatagggctacacagaaaaaccatatctcagaaaaaaaaaaagttccaaacacacacacacacacacacacacacacacacacacacacacacacacacacacacagcgcgccgcggcgatgaggggaagtcgtgcctaaaataaatatttttctggccaaagtgaaagcaaatcactatgaagaggtactcctagaaaaaataaatacaaacgggctttttaatcattccagcactgttttaatttaactctgaatttagtcttggaaaagggggcgggtgtgggtgagtgagggcgagcgagcagacgggcgggcgggcgggtgagtggccggcggcggtggcagcgagcaccagaaaacaacaaaccccaagcggtagagtgttttaaaaatgagacctaaatgtggtggaacggaggtcgccgccaccctcctcttccactgcttagatgctcccttccccttactgtgctcccttcccctaactgtgcctaactgtgcctgttccctcaccccgctgattcgccagcgacgtactttgacttcaagaacgattttgcctgttttcaccgctccctgtcatactttcgtttttgggtgcccgagtctagcccgttcgctatgttcgggcgggacgatggggaccgtttgtgccactcgggagaagtggtgggtgggtacgctgctccgtcgtgcgtgcgtgagtgccggaacctgagctcgggagaccctccggagagacagaatgagtgagtgaatgtggcggcgcgtgacggatctgtattggtttgtatggttgatcgagaccattgtcgggcgacacctagtggtgacaagtttcgggaacgctccaggcctctcaggttggtgacacaggagagggaagtgcctgtggtgaggcgaccagggtgacaggaggccgggcaagcaggcgggagcgtctcggagatggtgtcgtgtttaaggacggtctctaacaaggaggtcgtacagggagatggccaaagcagaccgagttgctgtacgcccttttgggaaaaatgctagggttggtggcaacgttactaggtcgaccagaaggcttaagtcctaccccccccccccttttttttttttttcctccagaagccctctcttgtccccgtcaccgggggcaccgtacatctgaggccgagaggacgcgatgggcccggcttccaagccggtgtggctcggccagctggcgcttcgggtctttttttttttttttttttttttttcctccagaagccttgtctgtcgctgtcaccgggggcgctgtacttctgaggccgagaggacgcgatgggccccggcttccaagccggtgtggctcggccagctggagcttcgggtctttttttttttttttttttttttttttctccagaagccttgtctgtcgctgtcaccgggggcgctgtacttctgaggccgagaggacgcgatgggtcggcttccaagccgatgtggcggggccagctggagcttcgggtttttttttttcctccagaagccctctcttgtccccgtcaccgggggcgctgtacttctgaggccgagaggacgtgatgggcccgggttccaggcggatgtcgcccggtcagctggagctttggatctttttttttttttttcctccagaagccctctcttgtccccgtcaccgggggcaccttacatctgagggcgagaggacgtgatgggtccggcttccaagccgatgtggcggggccagctggagcttcgggttttttttttttcctccagaagccctctcttgtccccgtcaccgggggcgctgtacttctgaggccgagaggacgtgatgggcccgggttccaggcggatgtcgcccggtcagctggagctttggatcatttttttttttccctccagaagccctctcttgtccccgtcaccgggggcaccgtacatctgaggccgagaggacacgatgggcctgtcttccaagccgatgtggcccggccagctggagcttcgggtctttttttttttttttcctccagaagccttgtctgtcgctgtcacccggggcgctgtacttctgaggccgagaggacgcgatgggcccggcttccaagccggtgtggctcggccagctggagcttcgggtctttttttttttttttttttttcctccagaaaccttgtctgtcgctgtcacccggggcgcttgtacttctgatgccgagaggacgcgatgggcccgtcttccaggccgatgtggcccggtcagctggagctttggatcttttttttttttttttcctccagaagccctctcttgtccccgtcaccgggggcaccttacatctgaggcctagaggacacgatgggcccgggttccaggccgatgtggcccggtcagctggagctttggatcttttttttttttttcttccagaagccctcttgtccccgtcaccggtggcactgtacatctgaggcggagaggacattatgggcccggcttccaatccgatgtggcccggtcagctggagctttggatcttattttttttttaattttttcttccagaagccctcttgtccctgtcaccggtggcacggtacatctgaggccgagaggacattatgggcccggcttccaggccgatgtggcccggtcagctggagctttggatctttttttttttttttcttttttcctccagaagccctctctgtccctgtcaccgggggccctgtacgtctgaggccgagggaaagctatgggcgcggttttctttcattgacctgtcggtcttatcagttctccgggttgtcagggtcgaccagttgttcctttgaggtccggttcttttcgttatggggtcatttttgggccacctccccaggtatgacttccaggcgtcgttgctcgcctgtcactttcctccctgtctcttttatgcttgtgatcttttctatctgttcctattggacctggagataggtactgacacgctgtcctttccctattaacactaaaggacactataaagagaccctttcgatttaaggctgttttgcttgtccagcctattctttttactggcttgggtctgtcgcggtgcctgaagctgtccccgagccacgcttcctgctttcccgggcttgctgcttgcgtgtgcttgctgtgggcagcttgtgacaactgggcgctgtgactttgctgcgtgtcagacgtttttcccgatttccccgaggtgtcgttgtcacacctgtcccggttggaatggtggagccagctgtggttgagggccaccttatttcggctcactttttttttttttttttctcttggagtcccgaacctccgctcttttctcttcccggtctttcttccacatgcctcccgagtgcatttctttttgttttttttctttttttttttttttttttggggaggtggagagtcccgagtacttcactcctgtctgtggtgtccaagtgttcatgccacgtgcctcccgagtgcacttttttttgtggcagtcgctcgttgtgttctcttgttctgtgtctgcccgtatcagtaactgtcttgccccgcgtgtaagacattcctatctcgcttgtttctcccgattgcgcgtcgttgctcactcttagatcgatgtggtgctccggagttctcttcgggccagggccaagccgcgccaggcgagggacggacattcatggcgaatggcggccgctcttctcgttctgccagcgggccctcgtctctccaccccatccgtctgccggtggtgtgtggaaggcaggggtgcggctctccggcccgacgctgccccgcgcgcacttttctcagtggttcgcgtggtccttgtggatgtgtgaggcgcccggttgtgccctcacgtgtttcactttggtcgtgtctcgcttgaccatgttcccagagtcggtggatgtggccggtggcgttgcatacccttcccgtctggtgtgtgcacgcgctgtttcttgtaagcgtcgaggtgctcctggagcgttccaggtttgtctcctaggtgcctgcttctgagctggtggtggcgctccccattccctggtgtgcctccggtgctccgtctggctgtgtgccttcccgtttgtgtctgagaagcccgtgagaggggggtcgaggagagaaggaggggcaagaccccccttcttcgtcgggtgaggcgcccaccccgcgactagtacgcctgtgcgtagggctggtgctgagcggtcgcggctggggttggaaagtttctcgagagactcattgctttcccgtggggagctttgagaggcctggctttcgggggggaccggttgcagggtctcccctgtccgcggatgctcagaatgcccttggaagagaaccttcctgttgccgcagacccccccgcgcggtcgcccgcgtgttggtcttctggtttccctgtgtgctcgtcgcatgcatcctctctcggtggccggggctcgtcggggttttgggtccgtcccgccctcagtgagaaagtttccttctctagctatcttccggaaagggtgcgggcttcttacggtctcgaggggtctctcccgaatggtcccctggagggctcgccccctgaccgcctcccgcgcgcgcagcgtttgctctctcgtctaccgcggcccgcggcctccccgctccgagttcggggagggatcacgcggggcagagcctgtctgtcgtcctgccgttgctgcggagcatgtggctcggcttgtgtggttggtggctggggagagggctccgtgcacacccccgcgtgcgcgtactttcctcccctcctgagggccgccgtgcggacggggtgtgggtaggcgacggtgggctcccgggtccccacccgtcttcccgtgcctcacccgtgccttccgtcgcgtgcgtccctctcgctcgcgtccacgactttggccgctcccgcgacggcggcctgcgccgcgcgtggtgcgtgctgtgtgcttctcgggctgtgtggttgtgtcgcctcgccccccccttcccgcggcagcgttcccacggctggcgaaatcgcgggagtcctccttcccctcctcggggtcgagagggtccgtgtctggcgttgattgatctcgctctcggggacgggaccgttctgtgggagaacggctgttggccgcgtccggcgcgacgtcggacgtggggacccactgccgctcgggggtcttcgtcggtaggcatcggtgtgtcggcatcggtctctctctcgtgtcggtgtcgcctcctcgggctcccggggggccgtcgtgtttcgggtcggctcggcgctgcaggtgtggtgggactgctcaggggagtggtgcagtgtgattcccgccggttttgcctcgcgtgccctgaccggtccgacgcccgagcggtctctcggtcccttgtgaggacccccttccgggaggggcccgtttcggccgcccttgccgtcgtcgccggccctcgttctgctgtgtcgttcccccctccccgctcgccgcagccggtcttttttcctctctccccccctctcctctgactgacccgtggccgtgctgtcggaccccccgcatgggggcggccgggcacgtacgcgtccgggcggtcaccggggtcttgggggggggccgaggggtaagaaagtcggctcggcgggcgggaggagctgtggtttggagggcgtcccggccccgcggccgtggcggtgtcttgcgcggtcttggagagggctgcgtgcgaggggaaaaggttgccccgcgagggcaaagggaaagaggctagcagtggtcattgtcccgacggtgtggtggtctgttggccgaggtgcgtctggggggctcgtccggccctgtcgtccgtcgggaaggcgcgtgttggggcctgccggagtgccgaggtgggtaccctggcggtgggattaaccccgcgcgcgtgtcccggtgtggcggtgggggctccggtcgatgtctacctccctctccccgaggtctcaggccttctccgcgcgggctctcggccctcccctcgttcctccctctcgcggggttcaagtcgctcgtcgacctcccctcctccgtccttccatctctcgcgcaatggcgccgcccgagttcacggtgggttcgtcctccgcctccgcttctcgccgggggctggccgctgtccggtctctcctgcccgacccccgttggcgtggtcttctctcgccggcttcgcggactcctggcttcgcccggagggtcagggggcttcccggttccccgacgttgcgcctcgctgctgtgtgcttggggggggcccgctgcggcctccgcccgcccgtgagcccctgccgcacccgccggtgtgcggtttcgcgccgcggtcagttgggccctggcgttgtgtcgcgtcgggagcgtgtccgcctcgcggcggctagacgcgggtgtcgccgggctccgacgggtggcctatccagggctcgcccccgccgacccccgcctgcccgtcccggtggtggtcgttggtgtggggagtgaatggtgctaccggtcattccctcccgcgtggtttgactgtctcgccggtgtcgcgcttctctttccgccaacccccacgccaacccaccaccctgctctcccggcccggtgcggtcgacgttccggctctcccgatgccgaggggttcgggatttgtgccggggacggaggggagagcgggtaagagaggtgtcggagagctgtcccggggcgacgctcgggttggctttgccgcgtgcgtgtgctcgcggacgggttttgtcggaccccgacggggtcggtccggccgcatgcactctcccgttccgcgcgagcgcccgcccggctcacccccggtttgtcctcccgcgaggctctccgccgccgccgcctcctcctcctctctcgcgctctctgtcccgcctggtcctgtcccacccccgacgctccgctcgcgcttccttacctggttgatcctgccaggtagcatatgcttgtctcaaagattaagccatgcatgtctaagtacgcacggccggtacagtgaaactgcgaatggctcattaaatcagttatggttcctttggtcgctcgctcctctcctacttggataactgtggtaattctagagctaatacatgccgacgggcgctgaccccccttcccggggggggatgcgtgcatttatcagatcaaaaccaacccggtgagctccctcccggctccggccgggggtcgggcgccggcggcttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagtcaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgactggcgatgcggcggcgttattcccatgacccgccgggcagcttccgggaaaccaaagtctttgggttccggggggagtatggttgcaaagctgaaacttaaaggaattgacggaagggcaccaccaggagtgggcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctgcgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcattaaacgggagactgtggaggagcggcggcgtggcccgctctccccgtcttgtgtgtgtcctcgccgggaggcgcgtgcgtcccgggtcccgtcgcccgcgtgtggagcgaggtgtctggagtgaggtgagagaaggggtgggtggggtcggtctgggtccgtctgggaccgcctccgatttcccctccccctcccctctccctcgtccggctctgacctcgccaccctaccgcggcggcggctgctcgcgggcgtcttgcctctttcccgtccggctcttccgtgtctacgaggggcggtacgtcgttacgggtttttgacccgtcccgggggcgttcggtcgtcggggcgcgcgctttgctctcccggcacccatccccgccgcggctctggcttttctacgttggctggggcggttgtcgcgtgtggggggatgtgagtgtcgcgtgtgggctcgcccgtcccgatgccacgcttttctggcctcgcgtgtcctccccgctcctgtcccgggtacctagctgtcgcgttccggcgcggaggtttaaggaccccgggggggtcgccctgccgcccccagggtcggggggcggtggggcccgtagggaagtcggtcgttcgggcggctctccctcagactccatgaccctcctccccccgctgccgccgttcccgaggcggcggtcgtgtgggggggtggatgtctggagccccctcgggcgccgtgggggcccgacccgcgccgccggcttgcccgatttccgcgggtcggtcctgtcggtgccggtcgtgggttcccgtgtcgttcccgtgtttttccgctcccgaccctttttttttcctcccccccacacgtgtctcgtttcgttcctgctggccggcctgaggctacccctcggtccatctgttctcctctctctccggggagaggagggcggtggtcgttgggggactgtgccgtcgtcagcacccgtgagttcgctcacacccgaaataccgatacgactcttagcggtggatcactcggctcgtgcgtcgatgaagaacgcagctagctgcgagaattaatgtgaattgcaggacacattgatcatcgacacttcgaacgcacttgcggccccgggttcctcccggggctacgcctgtctgagcgtcggttgacgatcaatcgcgtcacccgctgcggtgggtgctgcgcggctgggagtttgctcgcagggccaaccccccaacccgggtcgggccctccgtctcccgaagttcagacgtgtgggcggttgtcggtgtggcgcgcgcgcccgcgtcgcggagcctggtctcccccgcgcatccgcgctcgcggcttcttcccgctccgccgttcccgccctcgcccgtgcaccccggtcctggcctcgcgtcggcgcctcccggaccgctgcctcaccagtctttctcggtcccgtgccccgtgggaacccaccgcgcccccgtggcgcccgggggtgggcgcgtccgcatctgctctggtcgaggttggcggttgagggtgtgcgtgcgccgaggtggtggtcggtcccctgcggccgcggggttgtcggggtggcggtcgacgagggccggtcggtcgcctgcggtggttgtctgtgtgtgtttgggtcttgcgctgggggaggcggggtcgaccgctcgcggggttggcgcggtcgcccggcgccgcgcaccctccggcttgtgtggagggagagcgagggcgagaacggagagaggtggtatccccggtggcgttgcgagggagggtttggcgtcccgcgtccgtccgtccctccctccctcggtgggcgccttcgcgccgcacgcggccgctaggggcggtcggggcccgtggcccccgtggctcttcttcgtctccgcttctccttcacccgggcggtacccgctccggcgccggcccgcgggacgccgcggcgtccgtgcgccgatgcgagtcacccccgggtgttgcgagttcggggagggagagggcctcgctgacccgttgcgtcccggcttccctgggggggacccggcgtctgtgggctgtgcgtcccgggggttgcgtgtgagtaagatcctccacccccgccgccctcccctcccgccggcctctcggggaccccctgagacggttcgccggctcgtcctcccgtgccgccgggtgccgtctctttcccgcccgcctcctcgctctcttcttcccgcggctgggcgcgtgtcccccctttctgaccgcgacctcagatcagacgtggcgacccgctgaatttaagcatattagtcagcggaggaaaagaaactaaccaggattccctcagtaacggcgagtgaacagggaagagcccagcgccgaatccccgccgcgcgtcgcggcgtgggaaatgtggcgtacggaagacccactccccggcgccgctcgtggggggcccaagtccttctgatcgaggcccagcccgtggacggtgtgaggccggtagcggccccggcgcgccgggctcgggtcttcccggagtcgggttgcttgggaatgcagcccaaagcgggtggtaaactccatctaaggctaaataccggcacgagaccgatagtcaacaagtaccgtaagggaaagttgaaaagaactttgaagagagagttcaagagggcgtgaaaccgttaagaggtaaacgggtggggtccgcgcagtccgcccggaggattcaacccggcggcgcgcgtccggccgtgcccggtggtcccggcggatctttcccgctccccgttcctcccgacccctccacccgcgcgtcgttcccctcttcctccccgcgtccggcgcctccggcggcgggcgcggggggtggtgtggtggtggcgcgcgggcggggccgggggtggggtcggcgggggaccgcccccggccggcgaccggccgccgccgggcgcacttccaccgtggcggtgcgccgcgaccggctccgggacggccgggaaggcccggtggggaaggtggctcggggggggcggcgcgtctcagggcgcgccgaaccacctcaccccgagtgttacagccctccggccgcgctttcgccgaatcccggggccgaggaagccagatacccgtcgccgcgctctccctctccccccgtccgcctcccgggcgggcgtgggggtgggggccgggccgcccctcccacggcgcgaccgctctcccacccccctccgtcgcctctctcggggcccggtggggggcggggcggactgtccccagtgcgccccgggcgtcgtcgcgccgtcgggtcccggggggaccgtcggtcacgcgtctcccgacgaagccgagcgcacggggtcggcggcgatgtcggctacccacccgacccgtcttgaaacacggaccaaggagtctaacgcgtgcgcgagtcaggggctcgtccgaaagccgccgtggcgcaatgaaggtgaagggccccgcccgggggcccgaggtgggatcccgaggcctctccagtccgccgagggcgcaccaccggcccgtctcgcccgccgcgccggggaggtggagcacgagcgtacgcgttaggacccgaaagatggtgaactatgcttgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatagctggcgctctcgctcccgacgtacgcagttttatccggtaaagcgaatgattagaggtcttggggccgaaacgatctcaacctattctcaaactttaaatgggtaagaagcccggctcgctggcgtggagccgggcgtggaatgcgagtgcctagtgggccacttttggtaagcagaactggcgctgcgggatgaaccgaacgccgggttaaggcgcccgatgccgacgctcatcagaccccagaaaaggtgttggttgatatagacagcaggacggtggccatggaagtcggaatccgctaaggagtgtgtaacaactcacctgccgaatcaactagccctgaaaatggatggcgctggagcgtcgggcccatacccggccgtcgccgcagtcggaacggaacgggacgggagcggccgcgggtgcgcgtctctcggggtcgggggtgcgtggcgggggcccgtcccccgcctcccctccgcgcgccgggttcgcccccgcggcgtcgggccccgcggagcctacgccgcgacgagtaggagggccgctgcggtgagccttgaagcctagggcgcgggcccgggtggagccgccgcaggtgcagatcttggtggtagtagcaaatattcaaacgagaactttgaaggccgaagtggagaagggttccatgtgaacagcagttgaacatgggtcagtcggtcctgagagatgggcgagtgccgttccgaagggacgggcgatggcctccgttgccctcggccgatcgaaagggagtcgggttcagatccccgaatccggagtggcggagatgggcgccgcgaggccagtgcggtaacgcgaccgatcccggagaagccggcgggaggcctcggggagagttctcttttctttgtgaagggcagggcgccctggaatgggttcgccccgagagaggggcccgtgccttggaaagcgtcgcggttccggcggcgtccggtgagctctcgctggcccttgaaaatccgggggagagggtgtaaatctcgcgccgggccgtacccatatccgcagcaggtctccaaggtgaacagcctctggcatgttggaacaatgtaggtaagggaagtcggcaagccggatccgtaacttcgggataaggattggctctaagggctgggtcggtcgggctggggcgcgaagcggggctgggcgcgcgccgcggctggacgaggcgccgccgccctctcccacgtccggggagaccccccgtcctttccgcccgggcccgccctcccctcttccccgcggggccccgtcgtcccccgcgtcgtcgccacctctcttcccccctccttcttcccgtcggggggcgggtcgggggtcggcgcgcggcgcgggctccggggcggcgggtccaaccccgcgggggttccggagcgggaggaaccagcggtccccggtggggcggggggcccggacactcggggggccggcggcggcggcgactctggacgcgagccgggcccttcccgtggatcgcctcagctgcggcgggcgtcgcggccgctcccggggagcccggcgggtgccggcgcgggtcccctccccgcggggcctcgctccacccccccatcgcctctcccgaggtgcgtggcgggggcgggcgggcgtgtcccgcgcgtgtggggggaacctccgcgtcggtgttcccccgccgggtccgccccccgggccgcggttttccgcgcggcgcccccgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccggccccgtcctcgcgtcggggtcggggcacgccggcctcgcgggccgccggtgaaataccactactctcatcgttttttcactgacccggtgaggcgggggggcgagccccgaggggctctcgcttctggcgccaagcgtccgtcccgcgcgtgcgggcgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcaccaagcgttggattgttcacccactaatagggaacgtgagctgggtttagaccgtcgtgagacaggttagttttaccctactgatgatgtgttgttgccatggtaatcctgctcagtacgagaggaaccgcaggttcagacatttggtgtatgtgcttggctgaggagccaatggggcgaagctaccatctgtgggattatgactgaacgcctctaagtcagaatccgcccaagcggaacgatacggcagcgccgaaggagcctcggttggccccggatagccgggtccccgtccgtcccgctcggcggggtccccgcgtcgccccgcggcggcgcggggtctccccccgccgggcgtcgggaccggggtccggtgcggagagccgttcgtcttgggaaacggggtgcggccggaaagggggccgccctctcgcccgtcacgttgaacgcacgttcgtgtggaacctggcgctaaaccattcgtagacgacctgcttctgggtcggggtttcgtacgtagcagagcagctccctcgctgcgatctattgaaagtcagccctcgacacaagggtttgtctctgcgggctttcccgtcgcacgcccgctcgctcgcacgcgaccgtgtcgccgcccgggcgtcacgggggcggtcgcctcggcccccgcgcggttgcccgaacgaccgtgtggtggttgggggggggatcgtcttctcctccgtctcccgaggacggttcgtttctctttccccttccgtcgctctccttgggtgtgggagcctcgtgccgtcgcgaccgcggcctgccgtcgcctgccgccgcagccccttgccctccggccttggccaagccggagggcggaggagggggatcggcggcggcggcgaccgcggcgcggtgacgcacggtgggatccccatcctcggcgcgtccgtcggggacggccggttggaggggcgggaggggtttttcccgtgaacgccgcgttcggcgccaggcctctggcggccgggggggcgctctctccgcccgagcatccccactcccgcccctcctcttcgcgcgccgcggcggcgacgtgcgtacgaggggaggatgtcgcggtgtggaggcggagagggtccggcgcggcgcctcttccattttttcccccccaacttcggaggtcgaccagtactccgggcgacactttgttttttttttttcccccgatgctggaggtcgaccagatgtccgaaagtgtccccccccccccccccccccggcgcggagcggcggggccactctggactcttttttttttttttttttttttttttaaattcctggaacctttaggtcgaccagttgtccgtcttttactccttcatataggtcgaccagtactccgggtggtactttgtctttttctgaaaatcccagaggtcgaccagatatccgaaagtcctctctttccctttactcttccccacagcgattctcttttttttttttttttttggtgtgcctctttttgacttatatacatgtaaatagtgtgtacgtttatatacttataggaggaggtcgaccagtactccgggcgacactttgtttttttttttttttccaccgatgatggaggtcgaccagatgtccgaaagtgtcccgtcccccccctcccccccccgcgacgcggcgggctcactctggactcttttttttttttttttttttttttaaatttctggaaccttaaggtcgaccagttgtccgtctttcactcattcatataggtcgaccggtggtactttgtctttttctgaaaatcgcagaggtcgaccagatgtcagaaagtctggtggtcgataaattatctgatctagatttgtttttctgtttttcagttttgtgttgttttgtgttgttttgtgttgttttgttttgttttgttttgttttgttttgttttgttttgttttgttttgttttgtgttgtgttgtgttgtgttgtgttgggttgggttgggttgggttgggttgggttgggttgggttgggttgggttgtgttgtttggttttgtgttgtttggtgttgttggttttgttttgtttgctgttgttttgtgttttgcgggtcgaacagttgtccctaaccgagtttttttgtacacaaacatgcactttttttaaaataaatttttaaaataaatgcgaaaatcgaccaattatccctttccttctctctcttttttaaaaattttctttgtgtgtgtgtgtgtgtgtgtgtgtgtgtgtgtgcgtgtgtgtgtgtgtgtgcgtgcagcgtgcgcgcgctcgttttataaatacttataataataggtcgccgggtggtggtagcttcccggactccagaggcagaggcaggcagacttctgagttcgaggccagcctggtctacagaggaaccctgtctcgaaaaatgaaaataaatacatacatacatacatacatacatacatacatacatacatacatacatatgaggttgaccagttgtcaatcctttagaattttgtttttaattaatgtgatagagagatagataatagatagatggatagagtgatacaaatataggtttttttttcagtaaatatgaggttgattaaccacttttccctttttaggtttttttttttttcccctgtccatgtggttgctgggatttgaactcaggaccctggcaggtcaactggaaaacgtgttttctatatatataaatagtggtctgtctgctgtttgtttgtttgcttgcttgcttgcttgcttgcttgcttgcttgcttgcttttttttttcttctgagacagtatttctctgtgtaacctggtgccctgaaactcactctgtagaccagcctggcctcaatcgaactcagaaatcctcctgcctcttgtctacctcccaattttggagtaaaggtgtgctacaccactgcctggcattattatcattatcattattaattttattattagacagaacgaaatcaactagttggtcctgtttcgttaattcatttgaaattagttggaccaattagttggctggtttgggaggtttcttttgtttccgatttgggtgtttgtggggctggggatcaggtatctcaacggaatgcatgaaggttaaggtgagatggctcgatttttgtaaagattacttttcttagtctgaggaaaaaataaaataatattgggctacgtttcattgcttcatttctatttctctttctttctttctttctttcagataaggaggtcggccagttcctcctgccttctggaagatgtaggcattgcattgggaaaagcattgtttgagagatgtgctagtgaaccagagagtttggatgtcaagccgtataatgtttattacaatatagaaaagttctaacaaagtgatctttaacttttttttttttttttctccttctacttctacttgttctcactctgccaccaacgcgctttgtacattgaatgtgagctttgttttgcttaacagacatatattttttcttttggttttgcttgacatggtttccctttctatccgtgcagggttcccagacggccttttgagaataaaatgggaggccagaaccaaagtcttttgaataaagcaccacaactctaacctgtttggctgttttccttcccaaggcacagatctttcccagcatggaaaagcatgtagcagttgtaggacacactagacgagagcaccagatctcattgtgggtggttgtgaaccacccaccatgtggttgcctgggatttgaactcaggatcttcagaagacgagtcagggctctaaaccgatgagccatctctccagccctcctacattccttcttaaggcatgaatgatcccagcatgggaagacagtctgccctctttgtggtatatcaccatatactcaataaaataatgaaatgaatgaagtctccacgtatttatttcttcgagctatctaaattctctcacagcacctccccctcccccacactgcctttctccctatgtttgggtggggctgggggaggggtggggtgggggcagggatctgcatgtcttcttgcaggtctgtgaactatttgcgatggcctggttctctgaactgttgagccttgtctatccagaggctgactggctagttttctacctgaagtccctgagtgatgatttccctgtgaattc

SEQ ID NO: 6 - DNA, 175 bp, Mus Musculus
ctcccgcgcggcccccgtgttcgccgttcccgtggcgcggacaatgcggttgtgcgtccacgtgtgcgtgtccgtgcagtgccgttgtggagtgcctcgctctcctcctcctccccggcagcgttcccacggttggggaccaccggtgacctcgccctcttcgggcctggatccg

SEQ ID NO: 7 - DNA, 755 bp, Mus musculus
ggtctggtgggaattgttgacctcgctctcgggtgcggcctttggggaacggcggggtcggtcgtgcccggcgccggacgtgtgtcggggcccacttcccgctcgagggtggcggtggcggcggcgttggtagtctcccgtgttgcgtcttcccgggctcttggggggggtgccgtcgttttcggggccggcgttgcttggcttacgcaggcttggtttgggactgcctcaggagtcgtgggcggtgtgattcccgccggttttgcctcgcgtctgcctgctttgcctcgggtttgcttggttcgtgtctcgggagcggtggtttttttttttttcgggtcccggggagaggggtttttccgggggacgttcccgtcgccccctgccgccggtgggttttcgtttcgggctgtgttcgtttccccttccccgtttcgccgtcggttctccccggtcggtcggccctctccccggtcggtcgcccggccgtgctgccggacccccccttctgggggggatgcccgggcacgcacgcgtccgggcggccactgtggtccgggagctgctcggcaggcgggtgagccagttggaggggcgtcatgcccccgcgggctcccgtggccgacgcggcgtgttctttgggggggcctgtgcgtgcgggaaggctgcgcacgttgtcggtccttgcgagggaaagaggctttttttttttagggggtcgtccttcgtcgtcccgtcggcggtggatccggcct

SEQ ID NO: 8 - DNA, 463 bp, Mus musculus
ggccgaggtgcgtctgcgggttggggctcgtccggccccgtcgtcctccgggaaggcgtttagcgggtaccgtcgccgcgccgaggtgggcgcacgtcggtgagataaccccgagcgtgtttctggttgttggcggcgggggctccggtcgatgtcttcccctccccctctccccgaggccaggtcagcctccgcctgtgggcttcgtcggccgtctccccccccctcacgtccctcgcgagcgagcccgtccgttcgaccttccttccgccttccccccatctttccgcgctccgttggccccggggttttcacggcgccccccacgctcctccgcctctccgcccgtggtttggacgcctggttccggtctccccgccaaaccccggttgggttggtctccggccccggcttgctcttcgggtctcccaacccccggccggaagggttcgggggttccggg

SEQ ID NO: 9 - DNA, 378 bp, Mus musculus
ggattcttcaggattgaaacccaaaccggttcagtttcctttccggctccggccggggggggcggccccgggcggtttggtgagttagataacctcgggccgatcgcacgccccccgtggcggcgacgacccattcgaacgtctgccctatcaactttcgatggtagtcgatgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaagcag

SEQ ID NO: 10 - DNA, 378 bp, Mus musculus
gatccattggagggcaagtctggtgccagcagccgcggtaattccagctccaatagcgtatattaaagttgctgcagttaaaaagctcgtagttggatcttgggagcgggcgggcggtccgccgcgaggcgagtcaccgcccgtccccgccccttgcctctcggcgccccctcgatgctcttagctgagttgtcccgcggggcccgaagcgtttactttgaaaaaattagagttgtttcaaagcaggcccgagccgcctggataccgccagctaggaaataatggaataggaccgcggttcctattttgtttggttttcggaactgagcccatgattaagggaaacggccgggggcattcccttattgcgccccccta

SEQ ID NO: 11 - DNA, 719 bp, Mus musculus
ggatctttcccgctccccgttcctcccggcccctccacccgcgcgtctccccccttcttttcccctctccggaggggggggaggtgggggcgcgtgggcggggtcgggggtggggtcggcgggggaccgcccccggccggcaaaaggccgccgccgggcgcacttcaaccgtagcggtgcgccgcgaccggctacgagacggctgggaaggcccgacggggaatgtggctcggggggggcggcgcgtctcagggcgcgccgaaccacctcaccccgagtgttacagccctccggccgcgctttcgcggaatcccggggccgaggggaagcccgatacccgtcgccgcgcttttcccctccccccgtccgcctcccgggcgggcgtgggggtgggggccgggccgcccctcccacgcccgtggtttctctctctcccggtctcggccggtttgggggggggagcccggttgggggcggggcggactgtcctcagtgcgccccgggcgtcgtcgcgccgtcgggcccggggggttctctcggtcacgccgcccccgacgaagccgagcgcacggggtcggcggcgatgtcggctacccacccgacccgtcttgaaacacggaccaaggagtctaacgcgtgcgcgagtcaggggctcgcacgaaagccgccgtggcgcaatgaaggtgaagggccccgtccgggggcccgaggtgggatcc

SEQ ID NO: 12 - DNA, 685 bp, Mus musculus
cgaggcctctccagtccgccgagggcgcaccaccggcccgtctcgcccgccgcgtcggggaggtggagcacgagcgtacgcgttaggacccgaaagatggtgaactatgcctgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatagctggcgctctcgcaaccttcggaagcagttttatccgggtaaaggcggaatggattaggaggtcttggggccggaaacgatctcaaactatttctcaaactttaaatgggtaaggaagcccggctcgctggcgtggagccgggcgtggaatgcgagtgcctagtgggccacttttggtaagcagaactggcgctgcgggatgaaccgaacgccgggttaaggcgcccgatgccgacgctcatcagaccccagaaaaggtgttggttgatatagacagcaggacggtggccatggaagtcggaatccgctaaggagtgtgtaacaactcacctgccgaatcaactagccctgaaaatggatggcgctggagcgtcgggcccatacccggccgtcgccggcagtcggaacgggacgggacgggagcggccgc

SEQ ID NO: 13 - DNA, 5162 bp, Artificial Sequence
  Chimeric bacterial plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatctgcggccgcgtcgacggaattcagtggatccactagtaacggccgccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccctagagctcgctgatcagcctcgactgtgcctctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2003/0224522 A1
(43) Pub. Date: Dec. 4, 2003

(54) Methods for delivering nucleic acid molecules into cells and assessment thereof

(76) Inventors: Gary de Jong, North Vancouver (CA); Edward Perkins, Duluth, MN (US); Adele Telenius, Vancouver (CA); Neil MacDonald, Delta (CA)

Correspondence Address: Stephanie Seidman, Heller Ehrman White & McAuliffe LLP, 7th Floor, 4350 La Jolla Village Drive, San Diego, CA, 92122, US

(21) Appl. No.: 10/428,653

(22) Filed: May 1, 2003

Related U.S. Application Data
(60) Provisional application No. 60/377,547, filed on May 1, 2002.

(51) Int. Cl. C12N 15/88

(52) U.S. Cl. 435/458

(57) ABSTRACT

Methods for delivering nucleic acid molecules into cells and methods for measuring nucleic acid delivery into cells and the expression of the nucleic acids are provided. The methods are designed for introduction of large nucleic acid molecules, including artificial chromosomes, into cells.

### RELATED APPLICATIONS

Benefit of priority under 35 U.S.C. §119(e) is claimed to U.S. provisional application Serial No. 60/377,547 to Gary De Jong, Edward Perkins, Adele Telenius and Neil MacDonald, filed May 1, 2002, and entitled “METHODS FOR DELIVERING NUCLEIC ACID MOLECULES INTO CELLS AND ASSESSMENT THEREOF.”

This application is related to: U.S. application Ser. No. 09/815,979 entitled “METHODS FOR DELIVERING NUCLEIC ACID MOLECULES INTO CELLS AND ASSESSMENT THEREOF”, filed on Mar. 22, 2001, by De Jong et al.; U.S. application Ser. No. 09/815,981 entitled “METHODS FOR DELIVERING NUCLEIC ACID MOLECULES INTO CELLS AND ASSESSMENT THEREOF”, filed on Mar. 22, 2001, by De Jong et al.; U.S. application Ser. No. 10/086,745 entitled “METHODS FOR DELIVERING NUCLEIC ACID MOLECULES INTO CELLS AND ASSESSMENT THEREOF”, filed on Feb. 28, 2002, by De Jong et al.; PCT International Application No. PCT/US02/09262 entitled “METHODS FOR DELIVERING NUCLEIC ACID MOLECULES INTO CELLS AND ASSESSMENT THEREOF”, filed on Mar. 22, 2002, by De Jong et al.; International PCT application No. PCT/US03/xxxxx, entitled “METHODS FOR DELIVERING NUCLEIC ACID MOLECULES INTO CELLS AND ASSESSMENT THEREOF”, filed on May 1, 2003, by De Jong et al.

The subject matter of each of these applications is incorporated by reference in its entirety.

### FIELD OF THE INVENTION

The present invention relates to methods of delivering nucleic acid molecules into cells and methods for measuring nucleic acid delivery into cells and the expression of the nucleic acids therein.

### BACKGROUND

A number of methods for the delivery of nucleic acid molecules, particularly plasmid DNA and other small fragments of nucleic acid, into cells have been developed. Many of these methods are not ideal for delivery of larger nucleic acid molecules. Thus, there is a need for methods of delivering nucleic acid molecules of increasing size and complexity, such as artificial chromosomes, into cells. Methods are required for use with in vitro and in vivo procedures such as gene therapy and for production of transgenic animals and plants. Furthermore, there is a need for the ability to determine and assess the efficiency of delivery of DNA into cells.

### SUMMARY

In various aspects, methods for delivering nucleic acid molecules, particularly larger molecules, including artificial chromosomes, into cells, particularly synchronous cells, are provided. Methods for assessing delivery are also provided. Selected methods can be used to deliver nucleic acid molecules and/or proteins of a range of sizes, and can be particularly suitable for delivery of larger nucleic acid molecules, such as natural and artificial chromosomes with associated proteins and fragments thereof, into cells. Alternative methods can be designed for in vitro and ex vivo delivery of nucleic acid molecules for applications, including, but not limited to, delivery of nucleic acid molecules to cells for cell-based protein production, transgenic protein production and gene therapy. Methods of protein production in cells and in transgenic animals and plants, methods of introducing nucleic acid into cells to produce transgenic animals and plants, and methods for ex vivo and gene therapy are also provided.

Methods provided herein are designed for delivering a large nucleic acid molecule with or without associated proteins into a cell, but may also be used to deliver smaller molecules. Selected methods provided herein are designed for improving transfer of nucleic acid into the cells by delivering the nucleic acid into cells that are in a pre-selected phase. Cells of a pre-selected phase can be obtain in a number of ways. For example, cells can be isolated at a specific cell cycle stage using mechanical means such as elutriation or by exposing the cell to a cell cycle arrest agent or an agent that facilitates cell cycle synchronization. The agent can be one or more compositions, conditions and/or physical treatments that facilitates cell arrest and/or results in a synchronized population of cells. Selected agents and combinations thereof can include, for example, those that result in the highest transfection efficiency, highest delivery efficiency and/or the greatest amount of intact nucleic acid molecules transferred into the cell nucleus with an acceptable degree of cell survival. Thus, for example, the nucleic acid can be transferred to the cell at a phase or point in a phase of a cell that results in improved transfection efficiency, improved delivery efficiency and/or an improved proportion of intact nucleic acid molecules within the transfected cells (such improvements being relative to cells at a diffent phase or point in a phase of the cell cycle).

The selected methods may vary depending on the target cells (cells into which nucleic acid is delivered), the nucleic acid molecules, and methods used for obtaining cells in selected phases. Exemplary methods for delivery of large nucleic acid molecules into cells provided herein include obtaining a cell that is in a pre-selected phase of the cell cycle then contacting the cell with a large nucleic acid molecule. The pre-selected phase of the cell can be determined by introducing a large nucleic acid into cells at different phases and determining and comparing the efficiency of delivery and/or of transfection of the nucleic acid into the cells at different phases and selecting a phase at which the efficiency of delivery and/or of transfection is increased relative to efficiency of delivery and/or transfection at other phases.

The pre-selected phase can be obtained by exposing one or more cells to a cell cycle arrest agent, exposing the cell(s) to conditions that permit cell cycling, and then selecting a cell that is in the pre-selected phase. Arrest agents may include, but are not limited to anti-microtubule agents such as nocodazole and agents that effect DNA replication such as thymidine. The pre-selected phase can be any phase including G0, G1, S, G2, M, or at the interface of two phases including G0/G1, G2/M, G1/S, and S/G2. In an exemplary method, the phase is one in which the nuclear membrane of the cell is absent.

The methods provided herein can be adapted for delivery of large nucleic acid molecules into cells in a variety of environments for a variety of purposes. For example, nucleic acid molecules greater than about 0.6, 1, and 5 megabase pairs can be delivered into cells using the methods provided herein.

Included among the nucleic acid molecules that can be delivered into cells using alternative embodiments of the methods provided herein are natural chromosomes, artificial chromosomes, artificial chromosome expression systems (ACes), or fragments of any of these chromosomes, such as fragments that are greater than about 0.6 megabase or naked DNA that is greater than about 0.6 megabase.

In particular embodiments of the methods a delivery agent can be used. Delivery agents may include a cationic compound. Cationic compounds include, but are not limited to, a cationic lipid, a cationic polymer, a mixture of cationic lipids, a mixture of cationic polymers, a mixture of a cationic lipid and a cationic polymer and a mixture of a cationic lipid and a neutral lipid, polycationic lipids, non-liposomal forming lipids, activated dendrimers, pyridinium chloride surfactants, ethanolic cationic lipids, and cationic amphiphiles. Examples of cationic lipid compounds include N-[1-(2,3-dioleyloxy)propyl]-N,N,N-trimethylammonium chloride (DOTMA), dioleoylphosphatidylethanolamine (DOPE), 2,3-dioleyloxy-N-[2(spermine-carboxamido)ethyl]-N,N-dimethyl-1-propanaminiumtrifluoroacetate (DOSPA), C₅₂H₁₀₆N₆O₄4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O₄S₂.8CF₃CO₂H, C₄₁H₇₈NO₈P) C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, and (1-methyl-4-(1-octadec-9-enyl-nonadec-10-enylenyl) pyridinium chloride.

In particular embodiments of the methods the nucleic acid molecule can be exposed to an agent that increases contact between the nucleic acid molecule and the cell and the cell can be exposed to an agent that enhances permeability of the cell. The permeability enhancing agent may include energy such as ultrasound or electrical energy.

In alternative embodiments the methods can be used to introduce nucleic acids into a wide range of cell types, eukaryotic and prokaryotic, including cell lines, primary cells, primary cell lines, plant cells, and animal cells, including embryonic cells, nuclear transfer donor cells, stem cells, primary cells, immortalized cells, cells from immortalized cell lines, tumor cells, transformed cells, and cells that are capable of the generation of a specific organ. Animal cells may include mammalian cells, rodent cells or human cells. Exemplary cells for use in the methods provided herein include fibroblasts, synoviocytes, and fibroblast-like synoviocyte.

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, “nucleic acid” refers to a polynucleotide containing at least two covalently linked nucleotide or nucleotide analog subunits. A nucleic acid can be a deoxyribonucleic acid (DNA), a ribonucleic acid (RNA), or an analog of DNA or RNA. Nucleotide analogs are commercially available and methods of preparing polynucleotides containing such nucleotide analogs are known (Lin et al. (1994) Nucl. Acids Res. 22:5220-5234; Jellinek et al. (1995) Biochemistry 34:11363-11372; Pagratis et al. (1997) Nature Biotechnol. 15:68-73). The nucleic acid can be single-stranded, double-stranded, or a mixture thereof. For purposes herein, unless specified otherwise, the nucleic acid is double-stranded, or it is apparent from the context.

The term “nucleic acid” refers to single-stranded and/or double-stranded polynucleotides, such as deoxyribonucleic acid (DNA) and ribonucleic acid (RNA), as well as analogs or derivatives of either RNA or DNA. Also included in the term [symbol] nucleic acid [symbol] are analogs of nucleic acids such as peptide nucleic acid (PNA), phosphorothioate DNA, and other such analogs and derivatives.

As used herein, DNA is meant to include all types and sizes of DNA molecules including cDNA, plasmids and DNA including modified nucleotides and nucleotide analogs.

As used herein, nucleotides include nucleoside mono-, di-, and triphosphates. Nucleotides also include modified nucleotides, such as, but are not limited to, phosphorothioate nucleotides and deazapurine nucleotides and other nucleotide analogs.

As used herein, the term “large nucleic acid molecules” or “large nucleic acids” refers to a nucleic acid molecule of at least about 0.5 megabase pairs (Mbase) in size, greater than about 0.5 Mbase, including nucleic acid molecules at least about 0.6, 0.7, 0.8, 0.9, 1, 2, 3, 4, 5, 6, 7, 8, 9, 10, 20, 30, 40, 50, 60, 70, 80, 87, 90, and 100, 200, 300, or 500 Mbase in size. Large nucleic acid molecules can include proteins associated with the molecule. Large nucleic acid molecules typically can be on the order of about 10 to about 450 or more Mbase, and can be of various sizes, such as, for example, from about 250 to about 400 Mbase, about 150 to about 200 Mbase, about 90 to about 120 Mbase, about 60 to about 100 Mbase and about 15 to 50 Mbase.

Examples of large nucleic acid molecules include, but are not limited to, natural chromosomes and fragments thereof, especially mammalian chromosomes and fragments thereof which retain a centromere and telomeres, artificial chromosome expression systems (ACes; also called satellite DNA-based artificial chromosomes (SATACs); see U.S. Pat. Nos. 6,025,155 and 6,077,697), mammalian artificial chromosomes (MACs), plant artificial chromosomes, insect artificial chromosomes, avian artificial chromosomes and minichromosomes (see, e.g., U.S. Pat. Nos. 5,712,134, 5,891,691 and 5,288,625). The large nucleic acid molecules may include a single copy of a desired nucleic acid fragment encoding a particular nucleotide sequence, such as a gene of interest, or may carry multiple copies thereof or multiple genes or different heterologous sequences of nucleotides. For example, ACes can carry 40 or even more copies of a gene of interest. Large nucleic acid molecules can be associated with proteins. The protein can be any protein, including but not limited to, for example, chromosomal proteins, such as those that typically function to regulate gene expression and/or participate in determining overall structure and/or proteins that are not typically associated with chromosomes in a cell. Examples of a protein not typically associated with a chromosome include, but are not limited to, recombinant proteins that have been combined with a nucleic acid, for example a large nucleic acid, within or outside of a cell.

As used herein, a “functional fragment” of a chromosome refers to a fragment of a chromosome that retains the basic functional attributes of a chromosome. Thus, for example, a functional fragment of a chromosome can stably replicate and segregate in a cell in a manner similar to a chromosome from which the fragment is derived. A functional fragment of a chromosome can be generated in a number of ways. For example, a functional fragment can be generated by treating a complete, intact chromosome in such a way as to delete a portion or portions of the nucleic acid of the chromosome that are not required for the basic functioning of the chromosome in a cell.

As used herein, an artificial chromosome is a nucleic acid molecule that can stably replicate and segregate alongside endogenous chromosomes in a cell. It has the capacity to act as a gene delivery vehicle by accommodating and expressing foreign genes contained therein. A mammalian artificial chromosome (MAC) refers to chromosomes that have an active mammalian centromere(s). Plant artificial chromosomes, insect artificial chromosomes and avian artificial chromosomes refer to chromosomes that include plant, insect and avian centromeres, respectively. A human artificial chromosome (HAC) refers to chromosomes that include human centromeres. For exemplary artificial chromosomes, see, e.g., U.S. Pat. Nos. 6,025,155; 6,077,697; 5,288,625; 5,712,134; 5,695,967; 5,869,294; 5,891,691 and 5,721,118 and published International PCT application Nos, WO 97/40183 and WO 98/08964. An artificial chromosome can include proteins that are associated with the nucleic acid molecule.

As used herein, the term “satellite DNA-based artificial chromosome (SATAC)” is interchangeable with the term “artificial chromosome expression system (ACes)”. These artificial chromosomes are substantially all neutral non-coding sequences (heterochromatin) except for foreign heterologous, typically gene-encoding nucleic acid, that is interspersed within the heterochromatin for the expression therein (see U.S. Pat. Nos. 6,025,155 and 6,077,697 and International PCT application No. WO 97/40183). Foreign genes contained in these artificial chromosome expression systems can include, but are not limited to, nucleic acid that encodes traceable marker proteins (reporter genes), such as fluorescent proteins, such as green, blue or red fluorescent proteins (GFP, BFP and RFP, respectively), other reporter genes, such as a-galactosidase and proteins that confer drug resistance, such as a gene encoding hygromycin-resistance. Other examples of heterologous DNA include, but are not limited to, DNA that encodes therapeutically effective substances, such as anti-cancer agents, enzymes and hormones, and DNA that encodes other types of proteins, such as antibodies.

As used herein, the terms “heterologous” and “foreign” with reference to nucleic acids, such as DNA and RNA, are used interchangeably and refer to nucleic acid that does not occur naturally as part of a genome or cell in which it is present or which is found in a location(s) and/or in amounts in a genome or cell that differ from the location(s) and/or amounts in which it occurs in nature. It can be nucleic acid that is not endogenous to the cell and has been exogenously introduced into the cell. Examples of heterologous DNA include, but are not limited to, DNA that encodes a gene product or gene product(s) of interest introduced into cells, for example, for purposes of gene therapy, production of transgenic animals or for production of an encoded protein. Other examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies.

As used herein, “delivery,” which is used interchangeably with “transfection,” refers to the process by which exogenous nucleic acid molecules are transferred into a cell such that they are located inside the cell. Delivery of nucleic acids is a distinct process from expression of nucleic acids.

As used herein, “expression” refers to the process by which nucleic acid is translated into peptides or is transcribed into RNA, which, for example, can be translated into peptides, polypeptides or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

As used herein, cell recovery refers to a “total cell yield” after a specified time frame, which for purposes herein is twenty-four hours, and when used with reference to calculation of the clonal fraction.

As used herein, cell recovery time refers to a time frame in order for a cell to equilibrate to new conditions.

As used herein, cell survival refers to cell viability after a cytotoxic event, such as a delivery procedure.

As used herein, control plating efficiency (CPE) refers to the fraction of untreated cells, under standard optimal growth conditions for the particular cells, that survive a plating procedure. Plating efficiency refers to the fraction of treated cells that survive a plating procedure.

As used herein, clonal fraction is a measurement of cell recovery after delivery of exogenous nucleic acids into cells and the plating efficiency of the cells.

As used herein, transfer or delivery efficiency is the percentage of the total number of cells to which nucleic acids are delivered that contain delivered nucleic acid.

As used herein, transfection efficiency is the percentage of the total number of cells to which nucleic acids including a selectable marker are delivered that survive selection and/or express the selectable marker.

As used herein, index of potential transfection efficiency means the theoretical maximum transfection efficiency for a particular cell type under particular conditions, for example particular concentrations or amounts of particular delivery agents.

As used herein, the term “cell” is meant to include cells of all types, of eukaryotes and prokaryotes, including animals and plants.

As used herein, the term “cell cycle” includes the reproductive cycles through which a cell grows and divides to yield two or more daughter cells of the same or different ploidy. The cell cycle can be viewed as having several distinct phases, including, for example, in a eukaryotic organism, the G1, S, G2 and M phases. The M phase is the mitotic phase which includes segregation of the pairs of chromosomes, the production of two separate nuclei and division of the cell into two daughter cells. The M phase can be further divided into prophase, prometaphase, metaphase, anaphase, telophase and cytokinesis. In meiosis, similar phases occur in both the first and second cell divisions that typically yield four daughter cells. Typically, the G1, S and G2 phases are referred to as forming a period known as interphase during which the cell grows and progresses toward mitosis. During the S phase, DNA synthesis can occur in preparation for cell division.

As used herein, “phase” with reference to a cell refers to a state of a cell relative to the cell cycle. Thus, for example, the phase of a cell can be a phase within the active cell cycle (e.g., G1, S, G2 and M) or a phase out of the cell cycle, such as the resting phase referred to as GO in which the cell is quiescent and not actively passing through the cell cycle.

As used herein, “cell cycle arrest agent” refers to any means chemical, physical or others used to stop a cell from passing a set point or points in the growth cycle of a cell.

As used herein, “pre-selected phase” refers to a phase or point within a phase of a cell that specifically has been selected. The selection of a particular phase or point within a phase of a cell can be based on a determination of a characteristic of that point or phase. Thus, for example, the selection of a phase or point within a phase of a cell can be based on a determination that cells at that point or phase provide for improved transfer of a nucleic acid, particularly a large nucleic acid, into the cells relative to the transfer of the nucleic acid into cells at a different point or phase. Improved transfer can be, for example, any qualitative and/or quantitative improvement in any aspect of process and/or result of the transfer of the nucleic acid. Thus, improved transfer can be, for example, an increase in the efficiency of delivery and/or transfection of the nucleic acid into the cell, an increase in expression of the delivered nucleic acid into the cell and/or an increase in the integrity or intactness of the delivered nucleic acid in the cell.

As used herein, “conditions that permit cell cycling” refers to conditions that allow for growth of cells.

As used herein, a “synchronous population” with reference to a population of cells is one in which more cells in the population are in the same single phase than in an equivalent population of cells that has not been synchronized. Synchronization is effected by exposing a population of cells to conditions that result in an increase in the number of cells in the population that are in the same single phase. Such conditions are known by those of skill in the art and include but are not limited to natural and artificial treatments, such as, for example, changing any internal or external parameter of the cells, arresting cycling of the cells and releasing the cells from arrest, and physical separation of cells that are in different phases. An asynchronous population of cells is one which has not been exposed to any conditions that render the cells synchronous.

As used herein, “delivery agent” refers to compositions, conditions or physical treatments to which cells and/or nucleic acids can be exposed in the process of transferring nucleic acids to cells in order to facilitate nucleic acid delivery into cells. Delivery agents include compositions, conditions and physical treatments that enhance contact of nucleic acids with cells and/or increase the permeability of cells to nucleic acids. In general, nucleic acids are not directly treated with energy, such as sonoporation.

As used herein, cationic compounds are compounds that have polar groups that are positively charged at or around physiological pH. These compounds facilitate delivery of nucleic acid molecules into cells; it is thought this is achieved by virtue of their ability to neutralize the electrical charge of nucleic acids. Exemplary cationic compounds include, but are not limited to, cationic lipids or cationic polymers or mixtures thereof, with or without neutral lipids, polycationic lipids, non-liposomal forming lipids, ethanolic cationic lipids and cationic amphiphiles. Contemplated cationic compounds also include activated dendrimers, which are spherical cationic polyamidoamine polymers with a defined spherical architecture of charged amino groups which branch from a central core and which can interact with the negatively charged phosphate groups of nucleic acids (e.g., starburst dendrimers).

Cationic compounds for use as delivery agents also include mixtures of cationic compounds that include peptides and protein fragments. The additional components can be non-covalently or covalently bound to the cationic compound or otherwise associated with the cationic compound.

As used herein, ultrasound energy is meant to include sound waves (for external application) and lithotripter-generated shock waves (for internal application).

As used herein, electrical energy is meant to include the application of electric fields to cells so as to open pores in membranes for the delivery of molecules into the cell, e.g., electroporation techniques.

As used herein, cavitation compound is meant to include contrast agents that are typically used with ultrasound imaging devices and includes gas encapsulated and nongaseous agents. These cavitation compounds enhance the efficiency of energy delivery of acoustic or shock waves.

As used herein, “pharmaceutically acceptable” refers to compounds, compositions and dosage forms that are suitable for administration to the subject without causing excessive toxicity, irritation, allergic response or other undesirable complication.

As used herein, embryonic stem cells are primitive, immature cells that are precursors to stem cells.

As used herein, stem cells are primitive, immature cells that are precursors to mature, tissue specific cells.

As used herein, nuclear transfer donor cells are cells that are the source of nuclei, which are transferred to enucleated oocytes during the process of nuclear transfer.

As used herein, the term “subject” refers to animals, plants, insects, and birds into which the large DNA molecules can be introduced. Included are higher organisms, such as mammals and birds, including humans, primates, rodents, cattle, pigs, rabbits, goats, sheep, mice, rats, guinea pigs, cats, dogs, horses, chicken and others.

As used herein, “administering to a subject” is a procedure by which one or more delivery agents and/or large nucleic acid molecules, together or separately, are introduced into or applied onto a subject such that target cells which are present in the subject are eventually contacted with the agent and/or the large nucleic acid molecules.

As used herein, “applying to a subject” is a procedure by which target cells present in the subject are eventually contacted with energy such as ultrasound or electrical energy. Application is by any process by which energy can be applied.

As used herein, gene therapy involves the transfer or insertion of nucleic acid molecules, and, in particular, large nucleic acid molecules, into certain cells, which are also referred to as target cells, to produce specific gene products that are involved in correcting or modulating diseases or disorders. The nucleic acid is introduced into the selected target cells in a manner such that the nucleic acid is expressed and a product encoded thereby is produced. Alternatively, the nucleic acid may in some manner mediate expression of DNA that encodes a therapeutic product. This product can be a therapeutic compound, which is produced in therapeutically effective amounts or at a therapeutically useful time. It may also encode a product, such as a peptide or RNA, that in some manner mediates, directly or indirectly, expression of a therapeutic product. Expression of the nucleic acid by the target cells within an organism afflicted with a disease or disorder thereby provides a way to modulate the disease or disorder. The nucleic acid encoding the therapeutic product can be modified prior to introduction into the cells of the afflicted host in order to enhance or otherwise alter the product or expression thereof.

For use in gene therapy, cells can be transfected in vitro, followed by introduction of the transfected cells into the body of a subject. This is often referred to as ex vivo gene therapy. Alternatively, the cells can be transfected directly in vivo within the body of a subject.

As used herein, flow cytometry refers to processes that use a laser based instrument capable of analyzing and sorting out cells and or chromosomes based on size and fluorescence.

As used herein, a reporter gene includes any gene that expresses a detectable gene product, which can be RNA or protein. Preferred reporter genes are those that are readily detectable. Examples of reporter genes include, but are not limited to nucleic acid encoding a fluorescent protein, CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7: 725-737); bacterial luciferase (Engebrecht and Silverman (1984), PNAS 1: 4154-4158; Baldwin et al. (1984), Biochemistry 23: 3663-3667); and alkaline phosphatase (Toh et al. (1989) Eur. J. Biochem. 182: 231-238, Hall et al. (1983) J. Mol. Appl. Gen. 2: 101).

As used herein, a reporter gene construct is a DNA molecule that includes a reporter gene operatively linked to a transcriptional control sequence. The transcriptional control sequences include a promoter and other optional regulatory regions, such as enhancer sequences, that modulate the activity of the promoter, or control sequences that modulate the activity or efficiency of the RNA polymerase that recognizes the promoter, or control sequences that are recognized by effector molecules, including those that are specifically induced by interaction of an extracellular signal with a cell surface protein. For example, modulation of the activity of the promoter can be effected by altering the RNA polymerase binding to the promoter region, or, alternatively, by interfering with initiation of transcription or elongation of the mRNA. Such sequences are herein collectively referred to as transcriptional control elements or sequences. In addition, the construct can include sequences of nucleotides that alter translation of the resulting mRNA, thereby altering the amount of reporter gene product.

As used herein, promoter refers to the region of DNA that is upstream with respect to the direction of transcription of the transcription initiation site. It includes the RNA polymerase binding and transcription imitation sites and any other regions, including, but not limited to repressor or activator protein binding sites, calcium or cAMP responsive sites, and any such sequences of nucleotides known to those of skill in the art to alter the amount of transcription from the promoter, either directly or indirectly.

As used herein, a promoter that is regulated or mediated by the activity of a cell surface protein is a promoter whose activity changes when a cell is exposed to a particular extracellular signal by virtue of the presence of cell surface proteins whose activities are affected by the extracellular protein.

B. Cell Cycle

1. Cell Cycle Phases

In the reproductive cycle of a cell, the cell undergoes specific phases of DNA synthesis and division over a defined period of time. In a somatic mammalian cell, this time course is conventionally divided into four phases: G1 (first gap) phase, S (DNA synthesis) phase, G2 (second gap) phase, and M (mitotic) phase. Ninety percent or more of the total cell-cycle time course is dominated by the G1, S and G2 phases, collectively termed interphase. The remaining time is devoted to the M phase, during which the cell actually divides. For example, in rapidly proliferating cells of higher eukaryotes, M phases generally occur only once every 16 to 24 hours, and each M phase itself lasts only 1 to 2 hours. Cells in different tissues, in different species, and at different stages of embryonic development have division cycles that vary greatly in duration, from less than an hour (for example, in the early frog embryo) to more than a year (for example, in the adult human liver). Although all phases of the cell cycle vary to some extent, by far the greatest variation occurs in the duration of G1, which can be, in some cases, practically non-existant, or for such a length of time that the cell appears to have altogether ceased progressing through the divisional cycle and to have withdrawn into a quiescent state. Cells in such a quiescent G1 state are often said to be in the G0 state.

During the M phase or mitosis, the phase of cell division, the cell transitions through four substages: prophase, metaphase, anaphase, and telophase. In meiosis, similar substages occur in each of the two cellular division cycles that typically yield four daughter cells. Prophase involves the condensation of chromosomes and breakdown of nuclear membrane such that the nuclear contents are no longer separated from the cytoplasm. Metaphase is characterized by the migration of chromosomes to the equatorial plane of the cell. In anaphase, the chromosomes migrate to opposite poles of the cell segregating half sets of chromosomes to the two ends of the dividing cell. In late anaphase, constriction through the equator of the cell begins and results in the pinching off of two daughter cells in telophase. The daughter cells then begin interphase of a new cycle.

Meiosis typically includes two successive nuclear divisions with only one round of DNA replication. Four stages can be described for each nuclear division, similar to the stages of mitosis. The first division of meiosis can be divided into the following substages: prophase 1, when each chromosome dupicates and remains closely associated (called sister chromatids); metaphase 1, when homologous chromosomes align at the equatorial plate; anaphase 1, when homologous pairs separate with sister chromatids remaining together; and, telophase 1, when two daughter cells are formed with each daughter containing only one chromosome of the homologous pair. Similarly, The second division of meiosis can be divided into the following substages:prophase 2, when DNA does not replicate; metaphase 2, when chromosomes align at the equatorial plate; anaphase 2, when centromeres divide and sister chromatids migrate separately to each pole; and, telophase 2, when cell division is complete. The following detailed discussion relates largely to the mechanics of mitotic division, although it applies mutatis mutandis to cells undergoing meiosis.

Interphase starts with the G1 phase, in which the biosynthetic activities of the cells, which proceed very slowly during mitosis, resume at a high rate. During G1, the cell is subject to stimulation by extracellular mitogens and growth factors. In response to these stimuli, the cell passes through G1 and proceeds with DNA synthesis in S phase. The S phase begins when DNA synthesis starts, and ends when the DNA content of the nucleus has doubled and the chromosomes have replicated. The cell then enters the G2 phase, which continues until mitosis starts, initiating the M phase.

2. Cell Cycle Control

The regulation of the cell-cycle must ensure that the events in each phase are complete before moving to the next. To ensure completion of each phase, checkpoints for monitoring the integrity of DNA are strategically placed in G1 and at the G2/M interface to prevent progression and propagation of mutated or damaged cells. The G1 checkpoint can be described as the transition from early G1, when cells have the option of withdrawing from the cell cycle into the G0 state, and late G1, when cells are committed to another round of genome replication and division. In mammalian cells, this transition is also referred to as the “restriction point”. In response to growth and/or mitotic signals, the cell traverses the restriction point and commits to proceeding through the cell cycle. In the absence of such signals, the cell may undergo differentiation, apoptosis, or enter the quiescent state (G0). Once committed, the cell cycle is propelled by a series of cyclin/cyclin dependent protein kinase (CDK) complexes. Thus, the transition between cell cycle stages is delineated by the synthesis and subsequent proteolytic degradation of various cyclins.

For example, the mammalian cycle begins in G1 with increased expression of the D cyclins (D1, D2, D3). D cyclin activity has been linked to cell size and external growth-regulatory signals coupling cell size with entry into a new cell cycle. The D cyclins associate with CDK4 and CDK6 resulting in phosphorylation and activation of the CDKs. Activated CDK4/6 mediates a signal cascade resulting in the phosphorylation of the retinoblastoma protein and subsequent release of E2F transcription proteins and transcription of responder genes (including cyclin E). The responder proteins are required for G1 progression through the restriction point.

As the cell progresses through late G1, Cyclin E/CDK2 facilitates processes associated with the transition from G1 into S, primarily by shutting down the various braking systems that suppress S phase CDK activity in G1 . Cyclin E/CDK2 also initiates other early cell-cycle events, such as the duplication of the centrosome. Increased expression of cyclin A begins at the G1/S transition and continues through S phase. Cyclin A binding to CDK2 stimulates DNA synthesis. In late S phase cyclin A binds CDK1. As the cell approaches M phase, cyclin B expression increases and peaks at metaphase. Cyclin B/CDK1 along with Cyclin A/CDK1 activity propels the cell through mitosis. The degradation of these cyclins result in exit from mitosis and cytokinesis.

3. Chromosome Replication and Control

The synthesis of genomic DNA in S phase is only one component of the chromosomal replication process. A number of important processes throughout the cell cycle and specifically cell division are coupled to the DNA replication process and the proteins and factors which drive this process. In addition, many of the proteins and factors that are crucial for progression through the various cell cycle phases can be useful targets for cell arrest agents.

In eukaryotic cells, the DNA molecules of the chromosomes begin replication or synthesis at multiple start points or replication origins distributed along the individual chromosomes. Synthesis begins bidirectionally at these points and proceeds until the replication fork of adjacent start points meet. Although replication origins can vary greatly depending on cell type, they can be determined, in part, by the binding of the origin recognition complex (ORC), which has been conserved in evolution from yeast to humans. In most cells, one or more subunits of the ORC remain bound to origins through most of the cell cycle. For example, in multicellular eukaryotes, some ORC subunits remain bound throughout G1, S and G2 phases. Early in the G1 phase of the cell cycle, proteins required for DNA synthesis, including Cdc6, Cdt1 and six related proteins known as the MCM2-7 complex, are recruited to the ORC to form the prereplicative complex (pre-RC). By blocking the recruitment of such proteins, the cell can be arrested in G1/G0 phase. In late G1 phase, the pre-RC matures into a pre-initiation complex (pre-IC) by recruitment of additional factors including Cdc45, Sld3 and other factors involved in initiation and elongation during DNA synthesis.

At the end of G1, when the initiation complex at each replication origin is complete, a specific cyclin\CDK complex triggers the initiation of DNA synthesis and the S phase. In mammalian cells, this complex is cyclin A/CDK2. Another essential S phase promoting kinase is Cdc7 which interacts directly with the replication origin and/or ORC complex. After CDK and Cdc7 activation, origins are unwound and the heterotrimeric single-stranded DNA binding protein RPA is recruited. After early unwinding in early S phase, DNA polymerase á-primase complex is recruited to facilitate early synthesis. After initiation of early synthesis, additional DNA polymerases and auxiliary factors such as PCNA (proliferating cell nuclear antigen) are recruited to complete synthesis of each parental strand. As chromosomal DNA unwinds during synthesis, the topology of the DNA changes. Unwinding generates positive supercoils ahead of the replication fork and precatenanes in the newly replicated DNA behind the fork. The topology continues to change through mitosis. Agents which block the action or recruitment of required DNA synthesis machinery, such as, for example, DNA polymerase, ribonucleotide reductase or PCNA, will arrest the cell in S phase. PCNA also plays a central role in coupling many other cell cycle processes to the replication fork via direct protein protein interactions such as chromatid cohesion in late S phase and/or G2 phase. DNA damage and/or stalled replication forks trigger a global genome integrity checkpoint and recruitment of a number of kinases and other factors. Activation of this checkpoint prevents the firing of additional replication origins and prevents entry into mitosis, thus agents that target this checkpoint can be used to arrest cells in S and/or G2 phase.

In G2 phase, after DNA synthesis, the two daughter DNA molecules remain tightly associated with each other in a process known as sister chromatid cohesion. Cohesion is believed to occur discontinuously across the chromatids at specific and numerous cohesion sites, including centromeres. In the G2 phase, cohesion may help facilitate post-replicative repair of any double stranded breaks. Cohesion is also important for events occurring in M phase. For example, after assembly of a bipolar mitotic spindle in metaphase, controlled proteolysis of cohesions allows the two sister chromatids to be pulled to opposite poles and initiate anaphase. Attachment of the chromatids to microtubules (which form the mitotic spindle) from opposite poles by kinetochores is a crucial step in the segregation process and, therefore, factors which facilitate this process are useful targets for M phase cell arrest agents. Chromosome segregation coincides with cyclin B degradation which drives cytokinesis and mitotic exit.

C. Cell Phase-Based Delivery of Nucleic Acid into Cells

Provided herein are methods of delivering nucleic acids into cells in which the recipient cell is selected to be in a particular phase. The methods are particularly well suited for the delivery of large nucleic acids, including, for example, chromosomes and fragments thereof, and artificial chromosomes into cells. The methods utilize host cells that are in a phase that provide for transfer, and in particular improved transfer, of intact large nucleic acids into the cells. The phases or cycles of the cells in a host cell population that can be used in the methods can be synchronized to provide for an increased number of the cells that are in the same single phase for delivery of large nucleic acids into the cells. Methods for determining a cell phase for delivery of large nucleic acids into cells and methods for obtaining a cell or plurality of cells in a particular phase of the cell are also described herein.

1. Cells

The methods provided herein can be used in the delivery of nucleic acids into any cells, including, but not limited to, any eukaryotic and prokaryotic cells. Examples of cells that can be used in the methods include, but are not limited to, cell lines, primary cells, primary cell lines, plant cells and animal cells, including stem cells and embryonic cells. For example, fibroblasts, including lung and skin fibroblasts, fibroblast-like cells, synoviocytes, fibroblast-like synoviocytes, stem cells, including embryonic and adult stem cells, such as mesenchymal stem cells, myoblasts, lymphoblasts, carcinoma and hepatoma cells are among the many cells into which nucleic acids, and in particular large nucleic acids and artificial chromosomes, can be delivered and monitored using the methods provided herein. Particular cells include mammalian cells, for example, A9 cells (mouse fibroblasts, HPRT-; ATCC Accession no. CCL-1.4), CHO-S cells and DG44 cells (Chinese hamster ovary cells), V79 cells (Chinese hamster lung fibroblasts; ATCC Accession no. CCL-39), LMTK-cells (mouse fibroblasts; ATCC Accession No. CCL-1.3), skin fibroblasts (including primary human foreskin fibroblasts), L8 cells (rat myoblasts; ATCC Accession No. CRL-1769), CCD1043 SK cells (human fibroblasts; ATCC Accession No. CRL-2056), adult-derived mesenchymal stem cells (e.g., derived from human bone marrow; Cambrex Biosciences, East Rutherford, N.J.), synoviocytes (rat and human), Detroit 551 cells (human embryonic skin fibroblasts; ATCC Accession No. CCL-110), NSO (murine myeloma, ECACC Accession No. 85110503), 293 cells (human embryonic kidney cells transformed by type 5 (Ad 5) DNA (ATCC Accession No. CRL-1573), P46-Fl (bovine lymphocyte-like cell line), DT40 (chicken lymphoblasts), EJ30 cells (human bladder carcinoma), HepG2 cells (human hepatoma) and murine and bovine embryos.

In particular embodiments, the methods of delivery of nucleic acids into cells provided herein can be used in delivering nucleic acids into cells in order to treat a disease or disorder, e.g., in gene therapy applications. In gene therapy applications, the nucleic acid to be delivered into a cell may encode a therapeutic molecule, e.g., a protein. In a particular example, the protein can be Factor VIII. In many instances, successful gene therapy applications are complicated by a requirement that large nucleic acids be delivered into cells. It may also be desired to provide multiple copies of nucleic acid encoding one or more therapeutic molecules. Compounding the difficulties in gene therapy methods is the challenge that cells preferred for use in gene therapy applications are often not readily transfectable. The methods provided herein are particularly well suited for delivery of large nucleic acids, which can be in the form of artificial chromosomes or fragments thereof, into cells as can be used in therapeutic applications.

2. Nucleic Acids

Although any nucleic acid can be used in the cell phase-based methods described herein, the methods are particularly well suited for delivery of large nucleic acids into cells. In particular embodiments, the large nucleic acids are chromosomes, fragments thereof and artificial chromosomes and any other large and/or complex nucleic acid. In further particular embodiments, the large nucleic acid is associated with one or more proteins through any type of binding or other interaction. The methods described herein provide for improved transfer of such nucleic acids into cells. For example, the cell phase-based methods described herein may provide for delivery of large nucleic acid molecules into cells with greater transfection efficiency and greater integrity of the molecules than methods that do not include use of a cell that is in a pre-selected phase. The efficiency of transfection of large nucleic acid molecules, and in particular chromosomes, into cells using these cell phase-based methods is at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5 or at least 10-fold greater than the efficiency of transfection of the nucleic acid molecule to cells in an asynchronous population of cells.

In particular embodiments, the methods provide for the delivery of chromosomes, fragments thereof or artificial chromosomes into cells. Artificial chromosomes include Artificial Chromosome Expression System (ACes; also called satellite DNA-based artificial chromosomes (SATACs)) chromosomes, which are described, for example, in U.S. Pat. Nos. 6,025,155 and 6,077,697 and PCT International Patent Application Publication No. WO 97/40183, mammalian artificial chromosomes (MACs), plant artificial chromosomes, insect artificial chromosomes, avian artificial chromosomes and minichromosomes (see, e.g., U.S. Pat. Nos. 5,712,134, 5,891,691 and 5,288,625). For other exemplary artificial chromosomes, see, e.g., U.S. Pat. Nos. 5,695,967; 5,869,294; and 5,721,118 and published International PCT application No. WO 98/08964.

In methods for delivering chromosomes, functional fragments thereof or artificial chromosomes into cells as provided herein, the phase of the chromosome can be a consideration. During the cell cycle, chromosomes undergo morphological and other changes. For example, the topology of chromosomal DNA changes as it is unwound during DNA replication in the S phase of the cell cycle. Unwinding generates positive supercoils ahead of the replication fork and precatenanes in the newly replicated DNA behind the fork. During mitosis, chromosomes undergo condensation and decondensation with accompanying changes in appearance detectable through microscopy. Typically, chromosomes begin to condense during prophase and to decondense during telophase of mitosis. Thus, the condition of a chromosome differs at different phases of the cell cycle.

The condition of a chromosome can be a consideration first in isolating a chromosome from a cell and handling of the chromosome for subsequent transfer into a particular recipient cell. For example, condensation level can be a significant factor in the isolation and handling of chromosomes. Furthermore, the condition of a chromosome may impact its retention in a cell into which it is delivered. In particular methods provided herein for delivery of chromosomes or fragments thereof into cells, the phase of the chromosome or fragment thereof is the same or simliar to that of the recipient cell at the time of delivery. In further embodiments, the phase of the chromosome and of the recipient cell is the M phase, and, in particular, metaphase.

3. Determining Cell Cycle Phase to be Used in a Method for Delivering Nucleic Acid to a Cell

In methods provided herein for delivering nucleic acids to cells, a recipient cell is in a pre-selected phase. The pre-selected phase can be one that can provide for improved transfer of a nucleic acid, such as a large nucleic acid, into the cell. The pre-selected phase can differ for different types of recipient cells and for different types of nucleic acids and can be determined as described herein and using procedures known in the art. Improved transfer can take into account a number of factors. For example, delivery efficiency, transfection efficiency, integrity of the transferred molecules and cell viability after delivery are major considerations in determining improved transfer. Thus, for example, improved transfer can be an increased delivery or transfection efficiency attainable while still retaining intact transferred nucleic acids in a certain percentage of the recipient cells.

(a) Assessing Cell Phase and Chromosome Phase

One way to determine the phase of the cell cycle that a recipient cell should be in for delivery of the large nucleic acid into the cell involves determining and comparing the efficiencies of delivery and/or transfection, or comparing any other factor of relevance in achieving improved transfer, of nucleic acid into the cell in different phases of the cell cycle and selecting the cycle in which improved transfer of the nucleic acid is obtained. In order to compare the delivery and/or transfection efficiencies, or any other relevant parameter of transfer, the cell phases of the recipient cells are determined and the nucleic acid is contacted with the cells of the different phases.

Methods of assessing the phase of a cell or population of cells are described herein and are known in the art [see, e.g., Darzynkiewicz et al. (2001) Seminars Hematol. 38:179-193 and Nunez (2001) Curr. Issues Mol. Biol. 3:67-70]. Cell phase can be assessed in a variety of ways including cytometric analysis, microscopic analysis, gradient centrifugation, elutriation and fluorescence techniques including immunofluorescence (which can be used in combination with, for example, any of the preceding techniques). Cytometric techniques include exposing the cell to an agent or stain, such as DNA-binding dyes, e.g., propidium iodide (PI), and analyzing cellular DNA content by flow cytometry. Immunofluorescence techniques include detection of specific cell cycle indicators such as, for example, PCNA, thymidine analogs and cyclins, with fluorescent antibodies.

(i) Cytometric/Immunocytochemical Methods

Cell phase analysis by cytometry can be used to identify the distribution of cells in particular phases of the cell cycle, to determine the kinetics of progression through the phases and to monitor the molecular and functional mechanisms associated with the cell cycle, for example by immunohistochemical detection of components such as cyclins, inhibitors of cyclin-dependent kinases, cell cycle-associated protooncogenes and tumor suppressor genes. In one method of cell phase analysis using flow cytometry, the nuclear DNA content of a cell can be quantitatively measured at high speed as an indicator of cell cycle phase. DNA content is a marker of cell phase because the DNA content of a cell changes between the several phases of the cell cycle. Cells in G0/1 phase have DNA content set equal to 1 unit of DNA; cells in S phase duplicate DNA, increasing its content in proportion to progression through S; and upon entering G2 and then M phases, cells have twice the G0/1 phase DNA content (i.e., 2 units of DNA). Thus, S phase cells have a DNA content that is intermediate between that of cells in G1 and G2/M (which have twice as much DNA as cells in G1). Univariate analysis of cellular DNA content allows discrimination of G0/1, S and G2/M phase cells.

Flow cytometry measurement of cellular DNA content typically involves addition of a dye that binds stoichiometrically to DNA to a suspension of permeabilized cells or nuclei. Generally, cells are fixed or permeabilized, e.g., with a detergent, and then stained with a DNA-binding dye. Examples of such dyes include, but are not limited to, a nucleic acid-specific fluorochrome, propidium iodide (PI) or 4′ 6′-diamidino-2-phenylindole (DAPI). PI stains RNA in addition to DNA; thus, to avoid inclusion of measurement of fluorescence due to RNA in determining DNA content of a cell, it can be necessary to remove RNA by incubation with RNase. The DNA-bound PI emits red fluorescence when excited with blue light (488 nm). The DAPI-DNA complex can be excited by UV light (360 nm) and emits blue fluorescence. DNA can also be stained in live cells with the UV light-excitable fluorochrome Hoeschst 33242 which also emits blue fluorescence. Other DNA-binding dyes include, but are not limited to, Hoechst 33258, 7-AAD, LDS 751, and SYTO 16 (see, e.g., Molecular Probes Handbook of Fluorescent Probes and Research Chemicals, Haugland, Sixth Ed.; chapters 8 and 16 in particular).

In general, the DNA-binding dye is contacted with a cell (e.g., in concentrations ranging from about 1 lg/ml to about 5 lg/ml) and taken up passively by the cell. The dye is allowed to incubate for some period of time which can depend in part on the particular dye being used and can be determined empirically. Once inside the cell, the dye binds to DNA, for example by intercalation, although in some cases the dye can be a major or minor groove binding compound).

The stained material incorporates an amount of dye proportional to the amount of DNA. The stained material is then measured in the flow cytometer and the emitted fluorescent signal yields an electronic pulse with a height (amplitude) proportional to the total fluorescence emission from the cell. The results of fluorescence measurements can also be displayed as cellular DNA content frequency histograms which show the proportions of cells in the various phases of the cycle based on differences in fluorescence intensity. Software containing mathematical models that fit the DNA histogram of a singlet have been developed to calculate the percentages of cells occupying the different phases of the cell cycle. Several manufacturers provide software for cell cycle analysis, including, for example, Becton Dickinson (CellFit™).

Frequency histograms do not provide information about the rate of cell cycle progression or cell kinetics. The duration of a phase of the cell cycle (S;T_{S}), however, can be estimated if the duration of the cell cycle (T_{C}) or the doubling time of cells in culture is known. For example, the following equation can be used to estimate the duration of the S phase: T_{S}/T_{C}=In(F_{S}+1)/In2, where T_{S} is the duration of the S phase, T_{C} is duration of the cell cycle (or cell doubling timne in culture) and F_{S} is a fraction of the S phase cells estimated from the DNA content frequency histogram. This equation applies to cells growing exponentially, when both daughter cells subsequently divide.

Univariate DNA content analysis also cannot be used to distinguish noncycling, quiescent G0 cells (e.g., normal quiescent cells that have not been stimulated by a mitogenic signal to enter the cell cycle and stem cells that can remain in G0 for long periods of time) because G0 cells have the same DNA content as cycling G1 cells. One way to distinguish cells in G0 and G1 phases is based on differing RNA contents of cells in these phases. G0 cells have a low RNA content reflecting a low number of ribosomes in the cells. Furthermore, cells in early and late stages of the G1 phase contain differing amounts of RNA. Cells in early G1 or G1A, i.e., G1 cells in the growth phase, accumulate RNA and proteins but have an amount of RNA that is less than a threshold amount required in order for a cell to enter S phase. Thus, cells in the early G1 phase are referred to as a sub-threshold subpopulation. Cells in late G1 or G1B, which can enter the S phase without additional RNA or protein accumulation, have supra-threshold amounts of RNA.

Bivariate cytometric analysis of DNA versus RNA content of cells allows discrimination between G1 and G0 cells, as well as between G1A and G1B cells, based on differences in RNA content. One method for differentially staining DNA and RNA exploits the metachromatic property of the cationic fluorochrome acridine orange (AO), which is excitable by blue light. Acridine orange interacts with DNA under specific staining conditions (proper concentration, ionic strength and pH) such that the DNA-AO intercalative complexes in the cell fluoresce green (530 nm). Under the same conditions, the RNA-AO complexes emit re (>640 nm) fluorescence. Acridine orange staining thus differentially stains DNA and RNA (green vs. red, respectively), and can be used in conjunction with analysis by flow cytometers having a single-laser (488 nm) excitation. If a two-laser excitation cytometer is used in the analysis of cells for varying RNA content, DNA and RNA can be distinguished by differential staining with a combination of Hoechst 33242 and pyronin Y [see, e.g., Shapiro (1998) Cytometry 2:143-150].

Another way to distinguish noncycling and cycling cells is based on differential expression of numerous proteins, such as, for example, proliferation-associated proteins. Antibodies against these marker proteins can be used for immunocytochemical identification of noncycling and cycling cells. For example, the antibody Ki-67 detects a protein that is ubiquitous in cycling but frequently absent in noncycling cells [see, e.g., EndI et al. (2001) Meth. Cell Biol. 63:399-418]. The expression of protein reactive with Ki-67 varies during the cell cycle such that it is minimal in G1 cells and increases rapidly during S and G2 phases to reach a maximal amount in M cells. Proliferating cell nuclear antigen (PCNA), which is a co-factor of DNA polymerase Ä, is another proliferation-associated marker protein in cells. PCNA is specifically expressed in S-phase nuclei and is in the cytosol during G1 and G2/M [see, e.g., Larsen et al. (2001) Meth. Cell Biol. 63:419-432]. Cytometric analysis of an immunocytochemically detectable marker protein can be used in conjunction with DNA content analysis to reveal the cell cycle phase specificity of marker expression.

Univariate DNA content analysis also cannot be used to distinguish cells in G2 and M phases of the cell cycle, which have the same DNA content. There are, however, numerous markers for mitotic cells that have been adapted to cytometric methods that can be used to distinguish such cells from G2 phase cells. For example, histone H3 phosphorylation, which varies throughout the cell cycle, is a useful marker for mitotic cells. From prophase until telophase nearly all histone H3 molecules in chromatin are phosphorylated on serine-10, while during the remainder of the cell cycle only a small fraction of nucleosomes have phosphorylated H3. Antibodies that specifically recognize the phosphorylated epitope of histone H3 are available and can be used in immmunocytochemical detection of phosphorylated H3 in conjunction with flow cytometric methods [see, e.g., Juan et al. (2001) Meth. Cell Biol. 63:343-354]. Bivariate analysis of DNA content versus phosphorylated histone H3 provides for identification of all four phases of the cell cycle.

In another method of cell phase analysis using flow cytometry, cellular levels of cyclin proteins can serve as the basis for determining the phase of a cell and for distinguishing cells in different phases. Progression through the cell cycle is maintained by timely, sequential phosphorylation and dephosphorylation of several intracellular proteins. Cyclins are proteins involved in the cell cycle control system in cells which works by phosphorylation of proteins to active or inactive forms. cyclins bind to and activate cyclin-dependent kinases (cdks). Cyclin-dependent protein kinases influence the progression of the cell cycle by phosphorylating serine and threonine residues on select proteins that control the synthesis of cellular elements required for reproduction. Mammalian cells appear to contain separate cdks proteins that are specific for the G1 and G2 checkpoints through which a cell passes prior to progressing into the S and M phases, respectively.

Distinct classes of cyclins are synthesized during the G1 and G2 phases. In the normal vertebrate cell cycle control system, at least eight cyclins (cyclins A-H) and eight cdks proteins (cdc2 and CDK 2-8) are known. G1 cyclins (e.g., cyclins D and E) accumulate and bind cdks proteins during the G1 phase, and a different class of cyclins, mitotic cyclins, accumulate and bind cdks proteins during the G2 phase. For example, cell cycle progression can begin with induction of cyclin D; passage of the G1 checkpoint requires the binding of CDK2 protein with cyclin E; activation of DNA synthesis requires CDK2 protein binding with cyclin A, and entry into M phase requires formation of a complex between cdc2 and cyclin B [see, e.g., Merrill (1998) Meth. Cell Biol. 57:229-249].

The cellular level of cdks proteins is substantially invariable during the cell cycle. In contrast, cyclins are synthesized and degraded as the cell progresses through the cell cycle. Following synthesis, cyclins bind to cdks proteins promoting the phosphorylation of target proteins by the cdks. Subsequently, the cyclins are degraded, the cdks are released and target protein phosphorylation is reduced or ceased. The rate-limiting step in the binding reactions between cdks and cyclins appears to be the synthesis of the appropriate cyclin. Thus, the cellular content of some of the cyclins, for example, cylcins D, E, A and B, oscillates detectably during the cell cycle. Cyclin expression can be analyzed immunocytochemically (e.g., using fluorescently labeled anti-cyclin antibodies) by flow cytometry and used in distinguishing cells in different phases of the cell cycle based on bivariate analysis of cyclin expression versus DNA content. Correlation of DNA content with changes in cyclins permits visualization of nine subdivisions of the cell cycle [see, e.g., Darzynkiewicz et al. (1996) Cytometry 25:1-13].

Bivariate analysis of DNA content versus 5-bromo-2′-deoxyuridine 20 (BrdU) incorporation is another cytometric assay for assessing cell phase [see, e.g., Dolbeare et al. (1983) Proc. Natl. Acad. Sci. U.S.A. 80:5573-5577; Moran et al. (1985) J. Histochem. Cytochem. 33:821-827; Beisker et al. (1 987) Cytometry 8:235-239; Gray et al. (1990) “Quantitative cell cycle analysis,” in Flow Cytometry and Sorting, Melamed, Lindmo and Mendelsohn, eds., pp. 445-467; and Current Protocols in Cytometry (1997), Unit 7.7, John Wiley & Sons, Inc.]. BrdU is a thymidine analog that is incorporated into DNA during replication in cells exposed to the analog. DNA that has incorporated the analog can be detected immunocytochemically using fluorescein-tagged anti-BrdU antibodies. DNA content can be assessed, for example, by counterstaining with a red fluorescing intercalating fluorochrome such as, for example, PI or 7-aminoactinomycin D (7-AAD). Bivariate analysis of DNA content versus immunofluorescence of anti-BrdU antibody distinguishes S phase cells on the basis of their difference in DNA content from G1 or G2/M cells and also based on incorporation of the green fluorescing anti-BrdU antibodies.

(ii) Centrifugation/Elutriation-Based Methods

Centrifugation and centrifugal elutriation can be used to fractionate cells according to their size. Because cells in different phases differ in size, these methods can also be used to sort cells by cell phase and to thereby assess the phase of a cell. For example, early G1 phase cells are about half the size of mitotic or late G2 cells.

(iii) Chromosome Phase

Chromosomes undergo morphological, ultrastructural and topological changes during progression of the cell cycle. Thus, chromosomes from cells in different phases of the cell cycle can be distinctive. The topology of a chromosome differs at different phases of the cell cycle. Interphase chromosomal DNA exists in various decondensed states to facilitate gene expression. Chromatin in chromosomal regions that is not being transcribed exist predominently in the condensed form while regions being transcribed assume an extended form. Within S phase, chromosomal DNA is further dispersed as it unwinds during the replication process. Upon conclusion of S phase cohesion occurs to keep extended sister chromatids tightly associated. Typically, chromosomes begin to condense during prophase, undergoing several orders of supercoiling guided by histones and other facilitator proteins. Chromosomes are most dense during metaphase and begin to decondense again during telophase as the sister cells divide and normal transcription levels resume. Due to the various states in which chromosomal DNA exists throughout the cell cycle, when delivering DNA, particularly chromosomes, functional fragments thereof or artificial chromosomes into cells as provided herein, the phase of the isolated chromosome as well as the recipient cell chromosome can be a consideration in providing for improved transfer into the cells. Chromosomal phase phase can be determined by the methods described herein.

(b) Arresting Cell Cycle

(i) Agents and Methods of Arresting Cell Cycle

Cell arrest agents can be used to increase the amount of cells in a particular cell cycle phase within a population of cells or to arrest a particular cell at a particular phase. The agent may then be removed to allow the cell(s) to proceed through the cell cycle. Upon release from the agent, a population of cells will proceed more or less simultaneously through about one cell cycle. Arrest agents for use in the methods herein include compositions, conditions and physical treatments that arrest the cell cycle in a particular phase. Such agents include, but are not limited to, compounds and chemical compositions, peptides, proteins, temperature, light, pH, radiation and pressure.

Physical conditions, for example, may include but are not limited to starvation techniques such as nutrient, growth factor or hormone deprivation. Such techniques lead to an accumulation of cells in a particular stage, usually the G1 or G0 stage. Re-addition of nutrients or growth factors then allows the cells to re-enter the cell cycle in synchrony for about one generation. Most proliferating cell lines are known to respond to nutrient, growth factor or hormone deprivation in this manner. Two examples include the L929 mouse fibroblast and Nb2 rat lymphoma cell line. Culturing the L929 mouse fibroblast under serum starvation conditions (Glasgow Modified Minimal Essential Medium with 0.5% newborn calf serum and without glutamine) results in arrest of cells G0 phase. Upon replacing the starvation medium with normal growing medium, all cells progressed through the cell cycle in a synchronized manner (see Marenzi et al. (1999) Molec. Biol. Reports 26:261-267). The Nb2 rat lymphoma cell line is absolutely dependent on prolactin for stimulation of proliferation (see Gout et al. (1980) Cancer Res. 40:2433-2436). Culturing the cells in prolactin-deficient medium for 18-24 hours leads to arrest of proliferation, with cells accumulating early in the G1 phase of the cell cycle. Upon addition of prolactin, all the cells progress through the cell cycle until M phase at which point greater than 90% of the cells would be in mitosis. Deprivation techniques can be combined with additional arrest agents including other physical conditions, compounds or compositions, to increase the percentage of synchronized cells in the population. One or more agents can be used simultaneously or in succession to affect cell arrest. Temperature is another physical cell cycle arrest agent. For example, maintaining V79 cells at 25-32° C. results in an accumulation of cells in G0/G1. A further physical arrest agent is incubation under pressurized nitrous oxide which results in arrest at M phase (Brinkley and Rao (1973) J. Cell Biol. 58:96-106).

Cell arrest compounds for use in the methods provided herein are available commercially or can be synthesized by those of skill in the art. Cell arrest compounds typically target specific phases of the cell cycle. S phase arrest agents include, among others, agents that affect DNA replication or earlier phases through action on DNA polymerase and ribonucleotide reductase. Aphidicolin, 5-aminouracil (a thymidine analogue) thymidine, and hydroxyurea are examples of S phase arrest agents. Hydroxylurea disrupts S phase, for example, by destroying an essential tyrosine-free radical on the ribonucleotide reductase enzyme. Thymidine causes overaccumulation of thymidine triphosphate, which allosterically inhibits the reduction of cytidine diphosphate by ribonucleotide reductase, thus starving the replication forks for deoxycytidine triphosphate. Aphidicolin blocks replication by directly biding and inhibiting DNA polymerase. M phase cell arrest can be mediated by agents that affect specific mitotic events such as microtubule polymerization and spindle arrest. M phase arrest agents include, for example, nocodazole, colchicine, demecolcine (colcemide), oryzalin, propyzamide, trifluralin, 8-hydroxyquinoline, paradichlorobenzene, vinblastine, and a number of other glucosides, alkaloids, and coumarins (see Sharma, AK (1 956) Bot Rev 22:665-695; Sharma AK and Sharma A (1999) Plant Chromosomes: Analysis, Manipulation, and Engineering; Sharma AK (1999) Methods Cell Sci 21:73-78). Other arrest compounds for use in the methods provided herein include but are not limited to 1-â-D-Arabinofuranosylcytosine, olomoucine, roscovitine, 2-[N-(2-hydroxyethyl)]-N-(4-methoxybenzenesulfonyl)]amino-N-(4-chlorocinnamyl)-N-methylbenzylamine), daidzein, camptothecin, 5,6-Dichloro-1- [symbol] -D-ribofuanozylbenzimidazole, 5-fluorouracil, dexamethasone, acycloguanosine, calpain inhibitor I, cytosine arabinoside, CeReS-18, trichostatin A, indole-3-methanol, Staurosporine, tryprostatin B, and verruculogen.

Any cell arrest agent or combination of arrest agents can be used for enhancing a particular method of delivering nucleic acid molecules, such as DNA, into a particular cell type using the provided methods. The amount of agent and duration of exposure of a cell to the agent is dependent on the agent and cell type. These parameters can be readily determined for any agent and any cell by the methods provided herein.

In a particular embodiment of the method, one arrest agent is applied in an optimal amount and for an optimal duration to affect cell arrest. In another embodiment, one arrest agent is applied and removed in successive rounds or applied in different concentrations at different time points throughout the cell cycle to effect cell arrest. For example, using a “double block” protocol, cells can be incubated in replication-inhibiting levels of an arrest agent, such as thymidine, for sufficient time to let G2, M, and G1 cells collect in early S, then cells are incubated in low levels of agent for sufficient time to let all S phase cells complete replication, and finally cells are incubated again in high levels of agent for sufficient time to allow all the newly generated G2, M, and G1 cells to collect in early S phase. In another embodiment two or more arrest agents are applied simultaneously to effect cell arrest. In a further embodiment, two or more agents are applied successively to effect cell arrest. For example, to boost the yield of cells arrested in M phase, cells can be first exposed to an S phase arrest agent such as aphidicolin or thymidine. The S phase block can then be removed and the cells further incubated under mitosis-arresting conditions. After arresting with the S phase agent, a greater number of the cells in the population will be in a relatively late cell cycle phase, thus a greater number of cells will collect in M phase during the latter incubation.

One of skill in the art by using the provided optimization methods can readily determine which of the cell arrest agents or combination of agents are best suited for facilitating cell arrest and enhancing delivery of specific nucleic acid molecules, such as DNA, into a specific target cell type. These methods include determining the mean generation time, lengths of each phase of the cell cycle, fraction of cells proliferating in a cell population, arrest agent concentration, arrest agent incubation time, growth conditions, and nutrient conditions

Cell arrest protocols may include contacting cells with arrest agents for periods of time related to the duration of specific cell cycle phases, for example, the double block method described above. Thus, optimization may require knowledge of the duration of each cell cycle phase. To determine the duration of each cell cycle phase, determination of the mean generation time (length of one cell cycle) can be required. The mean generation time of cells in a population can be calculated from the change in cell number with incubation time. For example, cell number as a function of incubation time can be plotted for several different time points on semilog paper. A best fit line can be drawn through the data points and the time take for cell number to double can be calculated, yielding the mean generation time.

If the mean generation time is known, the duration of G1, S, G2, and M phase can be estimated. The duration of each cell cycle phase can be estimated, for example, by microscopy or flow cytometry. Microscopic methods may include autoradiography of labeled mitotic figures after pulse/chase labeling with [3H]thymidine (see e.g., Quastler and Sherman (1959) Exp. Cell Res. 17:420-438). When using flow cytometry, asynchronously growing cells can be stained with a DNA-intercalating dye such as propidium iodide, and fluorescence intensity can be used to determine the frequency of cells with a 2n, intermediate, or 4N complement of DNA. From a histogram of the DNA values, the phases can be determined manually or by computer algorithm (see e.g., Merrill, G. (1998) Methods Cell Biology; Cell Synchronization 57:229-249).

In addition, knowing the fraction of cells that are proliferating in the cell population can be useful for optimizing cell arrest conditions. Many methods of analyzing the average duration of a cell division cycle and its component phases assume that most cells (>95%) in the population are actively proliferating and that cell death is low (<5%). If a significant fraction of a population is quiescent, then the amount of quiescent cells may need to be considered when determining cell cycle kinetics of a cell population. To determine the fraction of quiescent cells in a population, the number of cells that fail to replicate their DNA during an interval corresponding to twice the mean generation time can be calculated. To determine the replicative fraction, [3H]thymidine incorporation followed by autoradiography or bromodeoxy-uridine(BUdr)incorporation followed by immunostaining can be used.

The optimum arrest agent concentration is the concentration that results in the greatest percentage of synchronized cells while maintaining acceptable levels of viability. Optimum arrest agent concentration can be determined by monitoring the dose response of a specific cell to a specific arrest agent. Dose response can be determined by titrating cells with an arrest agent and detecting cellular response. Cellular response can be detected, for example, by monitoring cell cycle phase and cell viability for each dose point over a period of one generation. Cell cycle phases can be monitored using cytometric analysis. In a specific example, for each dose point, cells are exposed to an agent or stain such as propidium iodide and the proportion of cells in G0 -G1, S, G2, and M are determined by flow cytometry. PI binds to the DNA within a cell and the amount of DNA contained in a cell is determined by comparing experimental PI measurements with control PI measurements. Any dye that incorporates into DNA can be used in a similar manner to PI. A cell that has not undergone DNA synthesis (S phase of the cell cycle) has 1 copy of the genome and is therefore in G0/G1. Once S phase is initiated the amount of DNA is greater than one copy and at the end of S phase the amount of DNA is at 2 copies and the cell is then in the G2/M stage of the cell cycle. Following nuclear and cellular division (M phase), each cell again has a single copy of the genome.

Arrest agent incubation time is a key factor for optimizing cell arrest. Excessively long incubations lead to poor synchrony or low viability. Insufficiently long incubations lead to poor synchrony because not all cells in the population reach the arrest state. Optimum arrest agent incubation time can be determined by exposing the cell to the agent and monitoring cell cycle phase over a period of one generation as described above. The optimum arrest agent incubation time is the time point at which the majority of the cells are arrested in the same phase while maintaining acceptable levels of viability.

Growth and nutrient conditions are dependent on cell type. Methods of determining growth and nutrient conditions are well known to those of skill in the art.

Meiosis regulating agents may also be used to obtain cells in a preselected phase. Meiosis regulating compounds are for example disclosed in U.S. Pat. Nos. 6,486,145; 6,407,086; and 5,716,777 (all of which are incorporated herein by reference).

(ii) Assessing Cell Cycle Arrest

Extent of cell cycle arrest can be determined using any methods of assessing cell phase as described herein or known in the art.

(c) Releasing Cells from Cell Cycle Arrest

In the methods provided herein for introducing nucleic acid molecules into cells, the nucleic acid molecules can be introduced into cells whose growth has been arrested, or into cells at a defined time point after release from cell cycle arrest. Release of cells from cell cycle arrest results in a synchronous population of cells, thus the defined time point corresponds to a particular phase of the cell cycle in the synchronous population of cells. Methods for releasing cells from cell cycle arest involve the restoration of normal growth conditions. Such methods are dependent on the cell arrest agent and type of cell used. For example, cells arrested by physical conditions such as nutrient, growth factor or hormone deprivation, are released from arrest by re-addition of the nutrient, growth factor, or hormone to the growth media. Cells arrested with a chemical agent can be released by removing the agent by washing and/or replacing the media as described, for example, in Example 10, or by the addition of an agent that counteracts or inhibits the arrest agent, for example, adding 2′-deoxycytidine 5 ′mono-phosophate (dCMP) to release cells from thymidine arrest. Upon restoration of normal growth conditions the cells re-enter the cell cycle in synchrony for about one generation depending on the cell type. The cells then gradually return to their logarithmically growing state. Re-entry into the cell cycle can be monitored by the methods described herein for monitoring cell cycle phase.

(d) Assessing Delivery Efficiency

Rapid Assessment Methods

Rapid delivery assessment methods based on automated, sensitive and accurate analysis procedures, such as flow cytometry, can be used to evaluate delivery efficiency. Such methods are less time-consuming and more accurate then manual detection of individual transfected cells by microscopic techniques. Using such rapid methods, it is possible to analyze nucleic acid molecule delivery data within 48 hours after transfection. Microscopic and colony formation analysis methods that can be used in evaluating stable nucleic acid molecule delivery rely on manual visualization or measurement of nucleic acid molecules (e.g., a selectable marker gene) expression, which is a distinct process from delivery. Such methods are associated with time delays in obtaining an assessment of the delivery method.

Data collected by flow cytometry analysis are statistically superior due to the ease at which large numbers of events, e.g., nucleic acid molecule transfer, are collected. The positive values obtained in these methods are instrument derived and therefore not as susceptible to judgment errors. Thus, these methods provide for greater accuracy in assessing nucleic acid molecule delivery. In contrast, microscopic analysis is limited by the time involved for scoring positive events and sample size is restrictive.

Because the methods of monitoring nucleic acid molecule delivery detect labeled nucleic acid molecules, such as DNA, and not a reporter gene expression product, it is possible to measure absolute values of nucleic acid molecules transferred, within twenty-four hours, without being hindered by cell autofluorescence and by the problems of differentiating wild-type cells from cells expressing low levels of reporter gene products (see, e.g., Ropp et al. (1995) Cytometry 21:309-317).

In rapid methods for monitoring nucleic acid molecule delivery, the nucleic acid molecules, such as DNA, to be delivered are labeled to allow for detection of the nucleic acid molecules in recipient cells after transfer into the cells. The nucleic acid molecules can be labeled by incorporation of nucleotide analogs. Any nucleic acid molecule analog that can be detected in a cell can be used in these methods. The analog is either directly detectable, such as by radioactivity, or can be detected upon binding of a detectable molecule to the analog that specifically recognizes the analog and distinguishes it from nucleotides that make up the endogenous nucleic acid molecules, such as DNA, within a recipient cell. Analogs that are directly detectable have intrinsic properties that allow them to be detected using standard analytical methods. Analogs may also be detectable upon binding to a detectable molecule, such as a labeled antibody that binds specifically to the analogs. The label on the antibody is one that can be detected using standard analytical methods. For example, the antibody can be fluorescent and be detectable by flow cytometry or microscopy.

In particular embodiments of these methods, the nucleic acid molecules, such as DNA, to be transferred is labeled with thymidine analogs, such as iododeoxyuridine (IdUrd) or Bromodeoxyuridine (BrdU). In preferred embodiments, IdUrd is used to label the nucleic acid molecules, such as DNA, to be transferred. The transferred IdUrd-labeled nucleic acid molecules, such as DNA, can be immunologically tagged using an FITC-conjugated anti-BrdU/IdUrd antibody and quantified by flow cytometry. Thus, the transfer of the labeled nucleic acid molecules, such as DNA, into recipient cells can be detected within hours after transfection.

Microscopy-Based Assessment Methods

Microscopic techniques for visualizing chromosome or plasmid transfer using bromodeoxyuridine (BrdU) (see. e.g., Pittman et al. J Immunol Methods 103:87-92 (1987)) are known in the art. These methods typically involve use of a large sample size to detect any low levels of transfer and further involve manual scoring.

Factors to Consider in Addressing Delivery of Nucleic Acids

Delivery of nucleic acids, including DNA, into cells is a process in which nucleic acids are transferred to the interior of a cell. Methods for the delivery of nucleic acids can be assessed in a variety of ways, including the following.

(i) Transfer or Delivery Efficiency

A delivery method can be assessed by determining the percentage of recipient cells in which the nucleic acids, including DNA, is present (i.e., the transfer efficiency). When evaluating a delivery method for the ultimate goal of generating cells that express the transferred nucleic acid, there are additional factors beyond mere presence of the nucleic acid in recipient cells that should be considered. Included among these additional factors is integrity of the nucleic acids and cell viability. When assessing a proliferating cell population, clonogenicity is the method of choice to measure viability. When the target cells population is non-dividing or slow growing, metabolic integrity can be monitored.

(ii) Clonogenicity

Clonogenicity represents a measure of the survivability of cells with respect to a delivery procedure, growth conditions and cell manipulations (e.g., plating). It is important to assess clonogenicity to determine whether a delivery procedure results in a sufficient number of viable cells to achieve a desired number of cells containing the transferred nucleic acid.

Clonogenicity can be expressed as a clonal fraction. The clonal fraction is an index that is calculated by multiplying two separate fractions and normalizing to a control plating efficiency correction factor (CPE). The two separate fractions that are multiplied in this calculation Oare the fraction of cells that survive a delivery procedure (population cell yield) and the fraction of cells that survive a plating procedure. The calculation is thus as follows:

$$Clonal Fraction = \frac{\# viable colonies after plating}{\# cell s plated} \times \frac{\# cell post \text{-} transfection}{\# cell transfected \times CPE}$$

The values used in this calculation for the number of cells post-transfection (i.e., post-delivery) and the number of colonies post-plating is based on cell or colony numbers at certain times in the process. For instance, the value for the number of cells post-transfection is representative of the number of cells at a time after nucleic acid delivery that is sufficient for the delivery process to be completed. This time can be determined empirically. Typically this time ranges from 4-48 hours and generally is about one day after transfection. Likewise, the value of the number of viable colonies post-plating is representative of the number of colonies at a time after nucleic acid delivery that is sufficient for the non-viable cells to be eliminated and the viable cells to be established as colonies. This time can be determined empirically. Typically this time ranges from that in which the average colony is made up of approximately 50 cells or generally is a time at which five cell cycles have passed.

A correction factor is included to take into account the plating efficiency of control wells, which is the ratio determined by the number of colonies counted divided by the number cells initially plated (typically 600-1000 cells). For LM(tk−) and V79-4 cells, the value of the correction factor typically ranges from about 0.7 to about 1.2 and can be, for example, 0.9.

The number of cells plated should remain constant at 1000 (simplified plating efficiency assay) done in duplicate, except in the case where the CPE is below 0.3, then number of cells seeded should be increased to a range of 5,000-50,000. If the CPE is below 0.1-0.2, then a viable fraction analysis should be considered.

(iii) Viable Fraction

If the target cell population is non-dividing or slowly dividing then reproductive or clonogenicity assays are not relevant. Less direct measurements of cell viability must be used to measure cell killing that monitor metabolic death rather than loss of reproductive capacity. These procedures include, for example: (1) membrane integrity as measured by dye exclusion, (2) inhibition of nucleic acid synthesis as measured by incorporation of nucleic acid precursors, (3) radioactive chromium release, and (4) MTT ASSAY (3-[4,5-dimethylthiazol-2-yl]-2,5-diphenyl-tetrazolium bromide). These methods are different from measurements of loss of proliferative capacity, as they reflect only immediate changes in metabolism, which can be reversed or delayed and hence lead to errors in estimation of cell viability. To minimize these errors, correlation of duplicate procedures is suggested.

(iv) Potential Transfection Efficiency (PTE) and Determination of Chromos Index (CI)

In assessing a delivery method used to transfer nucleic acids to cells with the goal of expression of the nucleic acids, including DNA, therein, it is desirable to obtain an indication of the theoretical maximum percentage of cells that are viable and contain the nucleic acid out of the total number of cells into which nucleic acids were delivered. This is referred to as the potential transfection efficiency and can be calculated from existing or historical experimental data sets and is determined as follows:follows:

$$\begin{matrix}Potential Transfection Efficiency ( PTE ) = Transfer Efficiency \times \\ ( Clonal Fraction or Viable Fraction ) \times \\ correction factor ( CF )\end{matrix}$$

The Chromos Index (C.I.) is an effective and rapid method to determine the Potential Transfection Efficiency of a proliferating population by using experimental values of % labeled nucleic acid, such as ACes, delivery to measure transfer efficiency and clonal fraction measured using a simplified clonogenicity assay.

Chromos Index (CI)=% labeled ACes delivery×estimated Clonal fraction×CF

The values of the transfer efficiency and of the clonal fraction and viable fraction are calculated as described above. The correction factor (CF) takes into account sample size, sample time and control plating efficiency. If all these factors are constant for each variable i.e., sampling time and size then the correction factor will approach the inverse of the value for the C.P.E., i.e., such that the clonal fraction or transfer efficiency can still approach 100% even with a low CF, or in other words, if delivery and viability are 100%, then the maximum potential transfection efficiency will equal the plating efficiency of the control cells. The calculation of C.I. allows for determination of each variable optimization, with the goal being for parameters, such as transfer efficiency, clonal fraction, and CF to approach one (or 100%). If sample size or time varies for either clonal fraction or transfer efficiency, then CF represents the extrapolated value based on slope or rate of change. An application of this assessment is provided in the EXAMPLES.

A stable transfection efficiency of about 1% is in the range (1-100%) that is considered useful for the introduction of large nucleic acid molecules into target cells. It is possible, using methods provided herein, to predict which delivery methods have to be selected for achieving desired transfection efficiencies without having to grow transfectants for extended times under selective conditions and determine numbers of cells surviving selection marker expression. This analysis involves calculation of the Chromos Index (CI) which integrates a “biological” value (the clonal fraction) with a measurement of chromosomal “uptake” or transfer efficiency (percentage of cells containing delivered ACes).

(e) Assessing Integrity of Transferred Nucleic Acid

Assessment of delivery of nucleic acid molecules may also be combined with an assessment of nucleic acid molecule, such as DNA, expression and integrity in recipient cells to provide even further information concerning the overall process of nucleic acid molecule transfer for purposes of expression.

It is of interest to evaluate the stability of the nucleic acid molecule, such as DNA, under the selected delivery conditions. Some delivery conditions and agents may have adverse effects on nucleic acid molecule structure. Furthermore, the labeling techniques used in certain methods of monitoring nucleic acid molecules, such as DNA, delivery may also impact nucleic acid molecules, such as DNA, structure and function.

The effects of delivery conditions on nucleic acid molecules can be assessed in a variety of ways, including microscopic analysis. In a particular exemplary analysis of the stability of artificial chromosomes, e.g., ACes, the chromosomes are exposed to the conditions of interest, e.g., IdU labeling, and analyzed under a fluorescent microscope for the ability to remain intact and condensed after incorporation of nucleotide analogs.

Analysis of the transfected cells can be performed to identify and assess the integrity of transferred nucleic acids. For example, the cells can be analyzed for indications of transferred nucleic acids such as artificial chromosomes or chromosomal segments, the presence of structures that may arise in connection with such nucleic acids and/or the presence of such nucleic acids. The size and structure of a transferred nucleic acid such as an artificial chromosome or ACes may also be determined. Analysis of the cells typically involves methods of visualizing nucleic acid structure and particularly chromosome structure, including, but not limited to, G- and C-banding and fluorescent in-situ hybridization (FISH) analyses using techniques described herein and/or known to those of skill in the art. Such analyses can employ specific labelling of particular nucleic acids, such as satellite DNA sequences, heterochromatin, rDNA sequences and heterologous nucleic acid sequences.

(f) Assessing Expression of Transferred Nucleic Acid

To facilitate analysis of nucleic acid molecules, such as DNA, expression, it is desirable to include in the transferred nucleic acid molecules, such as DNA, a reporter gene that encodes a readily detected product. For direct detection, such reporter gene products include, but are not limited to green fluorescent proteins (GFP), Red Fluorescent protein (RFP), luciferases, and CAT. For indirect detection, reporter gene products include, but are not limited, to â-galactosidase and cell surface markers.

By using, for example, artificial chromosomes such as ACes containing a GFP reporter gene, such as, but are not limited to, GFP coding sequences in combination with labeling of the ACes with DNA analogs, such as IdU, delivery and expression can be rapidly and accurately monitored. For example, following the delivery of IdU-labeled GFP gene-containing ACes to target cells by any of the described methods, the cells containing the ACes are split into two populations. One population is fixed and stained for IdU and analyzed by flow cytometry to determine percentage delivery. The other population is allowed to go through 4-5 cell divisions (approximately 72 hours), and the GFP fluorescence is measured as an indication of expression.

Such studies have revealed that incorporation of the analog label does not affect GFP protein expression, which indicates that the methods may be combined to monitor delivery and early expression of the ACes, thus providing more information to rapidly evaluate the efficiency of delivery methods. The combined methods can also be used to map the biological events between the initial stages of delivery and early gene expression.

(g) Cell Cycle Synchronization

The methods described herein provide for the transfection of a single cell or a population of two or more cells. To transfect a population of cells, it is often desirable to use a synchronous population of cells. A synchronous population of cells refers to a population in which a greater number of cells are in the same single phase compared to a corresponding asynchronous population. An asynchronous population of cells can be a population of cells in conditions that allow for normal exponential growth that have not been exposed to conditions that effect synchronization. Any method for obtaining a synchronous population of cells can be used in the methods described herein.

Methods for obtaining a synchronous population of cells may include isolating cells at a specific cell cycle stage using mechanical means such as mitotoic detachment or elutriation or chemical means such as exposing the cell to a cell cycle arrest agent. Mitotic detachment (or mitotic shake-off) is a minimally disruptive way of synchronizing monolayer cells. When most monolayer cells enter mitosis, they round up and become only slightly attached to the substratum of the culture plate. Because of their size and loose attachment, mitotic cells can be isolated by shaking and transferring the medium containing the detached cells to a fresh culture vessel. To generate large numbers of cells, a greter number of mother culture plates or vessels can be used.

Elutriation methods include sorting cells based upon morphological or biochemical features such as cell size, chromosome morphology, DNA content, base content, protein content (including cyclins), or other indicators of cell cycle phase such as enzymes or other factors that are expressed, degraded, activated or inactivated at defined phases of the cell cycle. Cells may also be sorted using combinations of the above cell cycle phase indicators. Combinations of cell cycle phase indicators can be used simultaneously or in consecutive sorting procedures.

Centrifugal elutriation is one method of sorting cells based on size and density. The system includes a specialized centrifuge rotor in which the centrifugal force and opposing bulk medium flow create a gradient, with smaller cells at the top and larger cells at the bottom. The rotor speed or medium flow is manipulated such that the gradient of size-separated cells is pushed toward the top and the small cells at the top of the gradient are eventually pushed out of the elutreation chamber and into a collection vessel. With further manipulation of the rotor speed and medium flow, progressively larger cells are pushed out of the elutriation chamber. Since G1 cells are roughly half the size of mitotic or late G2 cells, centrifugal elutreation can be used to fractionate cells according to their position in the cell cycle. Unlike the mitotic shake-off procedure, centrifugal elutriation can be used to synchronize both monolayer and suspension culture cells.

Cell phase sorting can also be accomplished through the use of a flow cytometer and sorter such as the fluorescence-activated cell sorter (FACS) as described herein. After elutriation the number and volume of cells in different fractions can be assessed using a multichannel cell analyzer. An advantage of using physical means to create a synchronized population of cells is that the cells do not spend any time in a growth-arrested state and therefore, anabolic activity remains relatively constant. Such methods also avoid the possible side-effects from treament of cells with chemicals.

Alternatively, cell synchronization agents can be used alone or in combination with physical sorting means to enhance a synchronous population of cells. Cell cycle arrest agents, as described herein, act by blocking the progression at a defined stage of the cell cycle which results in the accumulation of cells within the defined stage. Upon release from the arrest agent, the cells progress through the cell cycle in synchrony. When used in combination with cell sorting techniques, the cells can be sorted prior to or after release from the arrest agent.

The percentage of cell cycle synchronization within a population can be determined at different time points by the methods described above. The percentage of cell cycle synchronization is a measure of the percentage of cells cycling together in any one given phase at any one time point. The maximum percentage of cell cycle synchronization achievable is 100% of the cell population cycling together in any one phase. The percentage of cell cycle synchronization achievable using the cell cycle sorting and/or arrest and release methods described herein is at least 50%, at least 60%, at least 70%, at least 75%, at least 80%, at least 85%, at least 90%, at least 95% of cells cycling together in a particular phase.

D. Methods for the Delivery of DNA into Cells

In methods provided herein for delivering nucleic acids to cells, a recipient cell in a pre-selected phase is contacted with a nucleic acid molecule, such as a large nucleic acid molecule. The contacting can be conducted in a variety of ways under a variety of conditions. Conditions and procedures can be determined, for example, by comparing the delivery and/or transfection efficiencies (as can be calculated using methods described herein) achieved under various conditions.

The conditions used for delivering particular nucleic acid molecules, such as DNA, to recipient cells can depend on the particular nucleic acid molecule being transferred and the particular recipient cell. Particular conditions are those that result in the greatest amount of nucleic acid molecules, such as DNA, transferred into the cell nucleus with an acceptable degree of cell survival. Suitable conditions for delivery of particular pairings of nucleic acid molecules, such as DNA, and recipient cells can be determined using methods of monitoring nucleic acid molecules, such as DNA, delivery and methods of screening agents and conditions as provided herein or can be determined empirically using methods known to those of skill in the art.

In determining which conditions to use for contacting a nucleic acid with a cell in a pre-selected phase, a number of parameters can be considered. A method for detection of delivered nucleic acid is provided. This method, which can be used for assessing delivery of any nucleic acid molecule, can be used as a rapid screening tool to optimize nucleic acid, e.g., chromosome, transfer conditions.

In particular, delivery conditions can first be assessed for the ability to transfer nucleic acid molecules, such as DNA, into cells and to identify methods that provide a sufficient number of viable cells that express the transferred nucleic acid molecules, such as DNA. Once such conditions are identified, they can be optimized using delivery monitoring methods provided herein or known in the art and then assessed for the ability to provide for expression of the transferred nucleic acid molecules.

1. Naked DNA

“Naked” nucleic acid can be contacted with the recipient cell in the absence of any delivery agent or other procedures designed to facilitate transfer of nucleic acids into cells.

2. Microinjection

Nucleic acids can be delivered into cells through microinjection. Procedures for microinjection are known in the art.

3. Delivery Agents

Delivery agents include compositions, conditions and physical treatments that enhance contact of nucleic acid molecules, such as DNA, with cells and/or increase the permeability of cells to nucleic acid molecules, such as DNA. Such agents include, but are not limited to, cationic compounds, peptides, proteins, energy, for example ultrasound energy and electric fields, and cavitation compounds.

Delivery agents for use in the methods provided herein include compositions, conditions or physical treatments to which cells and/or nucleic acid molecules, such as DNA, can be exposed in the process of transferring nucleic acid molecules, such as DNA, to cells in order to facilitate nucleic acid molecules, such as DNA, delivery into cells. For example, compounds and chemical compositions, including, but not limited to, calcium phosphate, DMSO, glycerol, chloroquine, sodium butyrate, polybrene and DEAE-dextran, peptides, proteins, temperature, light, pH, radiation and pressure are all possible delivery agents.

(a) Cationic Compounds

Cationic compounds for use in the methods provided herein are available commercially or can be synthesized by those of skill in the art. Any cationic compound can used for delivery of nucleic acid molecules, such as DNA, into a particular cell type using the provided methods. One of skill in the art by using the provided screening procedures can readily determine which of the cationic compounds are best suited for delivery of specific nucleic acid molecules, such as DNA, into a specific target cell type.

(i) Cationic Lipids

Cationic lipid reagents can be classified into two general categories based on the number of positive charges in the lipid headgroup; either a single positive charge or multiple positive charges, usually up to 5. Cationic lipids are often mixed with neutral lipids prior to use as delivery agents. Neutral lipids include, but are not limited to, lecithins; phosphatidylethanolamine; phosphatidylethanolamines, such as DOPE (dioleoylphosphatidylethanolamine), DPPE (dipalmitoylphosphatidylethanolamine), POPE (palmitoyloleoylphosphatidylethanolamine) and distearoylphosphatidylethanolamine; phosphatidylcholine; phosphatidylcholines, such as DOPC (dioleoylphosphatidylcholine), DPPC (dipalmitoylphosphatidylcholine), POPC (palmitoyloleoylphosphatidylcholine) and distearoyl-phosphatidylcholine; fatty acid esters; glycerol esters; sphingolipids; cardiolipin; cerebrosides; and ceramides; and mixtures thereof. Neutral lipids also include cholesterol and other 3 [symbol] OH-sterols.

Other lipids contemplated herein, include: phosphatidylglycerol; phosphatidylglycerols, such as DOPG (dioleoylphosphatidylglycerol), DPPG (dipalmitoylphosphatidylglycerol), and distearoylphosphatidylglycerol; phosphatidylserine; phosphatidylserines, such as dioleoyl- or dipalmitoylphosphatidylserine and diphosphatidylglycerols.

Examples of cationic lipid compounds include, but are not limited to: Lipofectin (Life Technologies, Inc., Burlington, Ont.)(1:1 (w/w) formulation of the cationic lipid N-[1-(2,3-dioleyloxy)propyl]-N,N,N-trimethylammonium chloride (DOTMA) and dioleoylphosphatidylethanolamine (DOPE)); LipofectAMINE (Life Technologies, Burlington, Ont., see U.S. Pat. No. 5,334,761) (3:1 (w/w) formulation of polycationic lipid 2,3-dioleyloxy-N-[2(spermine-carboxamido)ethyl]-N,N-dimethyl-1-propanaminiumtrifluoroacetate (DOSPA) and dioleoylphosphatidylethanolamine (DOPE)), LipofectAMINE PLUS (Life Technologies, Burlington, Ont. see U.S. Pat. Nos. 5,334,761 and 5,736,392; see, also U.S. Pat. No. 6,051,429) (LipofectAmine and Plus reagent), LipofectAMINE 2000 (Life Technologies, Burlington, Ont.; see also International PCT application No. WO 00/27795) (Cationic lipid), Effectene (Qiagen, Inc., Mississauga, Ontario) (Non liposomal lipid formulation), Metafectene (Biontex, Munich, Germany) (Polycationic lipid), Eu-fectins (Promega Biosciences, Inc., San Luis Obispo, Calif.) (ethanolic cationic lipids numbers 1 through 12: C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀2N₆O₃.4CF₃CO₂H, C₄₄H₈₉N₅O₃.2CF₃CO₂H, C₁₀₀H₂₀₆N₁₂O₄S₂.8CF₃CO₂H, C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, C₄₁H₇₈NO₈P); Cytofectene (Bio-Rad, Hercules, Calif.) (mixture of a cationic lipid and a neutral lipid), GenePORTER (Gene Therapy Systems Inc., San Diego, Calif.) (formulation of a neutral lipid (Dope) and a cationic lipid) and FuGENE 6 (Roche Molecular Biochemicals, Indianapolis, Ind.) (Multi-component lipid based non-liposomal reagent).

(ii) Non-Lipid Cationic Compounds

Non-lipid cationic reagents include, but are not limited to SUPERFECT [symbol] (Qiagen, Inc., Mississauga, ON) (Activated dendrimer (cationic polymer:charged amino groups) and CLONfectin [symbol] (Cationic amphiphile N-t-butyl-N′-tetradecyl-3-tetradecyl-aminopropionamidine) (Clontech, Palo Alto, Calif.).

Pyridinium amphiphiles are double-chained pyridinium compounds, which are essentially nontoxic toward cells and exhibit little cellular preference for the ability to transfect cells. Examples of a pyridinium amphiphiles are the pyridinium chloride surfactants such as SAINT-2 (1-methyl-4-(1-octadec-9-enyl-nonadec-10-enylenyl) pyridinium chloride) (see, e.g., van der Woude et al. (1997) Proc. Natl. Acad. Sci. U.S.A. 94:1160). The pyridinium chloride surfactants are typically mixed with neutral helper lipid compounds, such as dioleoylphosphatidylethanolamine (DOPE), in a 1:1 molar ratio. Other Saint derivatives of different chain lengths, state of saturation and head groups can be made by those of skill in the art and are within the scope of the present methods.

(b) Energy

Delivery agents also include treatment or exposure of the cell and/or nucleic acid molecules, but generally the cells, to sources of energy, such as sound and electrical energy.

(i) Ultrasound

For in vitro and in vivo transfection, the ultrasound source should be capable of providing frequency and energy outputs suitable for promoting transfection. For example, the output device can generate ultrasound energy in the frequency range of 20 kHz to about 1 MHz. The power of the ultrasound energy can be, for example, in the range from about 0.05 w/cm2 to 2 w/cm2, or from about 0.1 w/cm2 to about 1 w/cm2. The ultrasound can be administered in one continuous pulse or can be administered as two or more intermittent pulses, which can be the same or can vary in time and intensity.

Ultrasound energy can be applied to the body locally or ultrasound-based extracorporeal shock wave lithotripsy can be used for “in-depth” application. The ultrasound energy can be applied to the body of a subject using various ultrasound devices. In general, ultrasound can be administered by direct contact using standard or specially made ultrasound imaging probes or ultrasound needles with or without the use of other medical devices, such as scopes, catheters and surgical tools, or through ultrasound baths with the tissue or organ partially or completely surrounded by a fluid medium. The source of ultrasound can be external to the subject's body, such as an ultrasound probe applied to the subject's skin which projects the ultrasound into the subject 's body, or internal, such as a catheter having an ultrasound transducer which is placed inside the subject 's body. Suitable ultrasound systems are known (see, e.g., International PCT application No. WO 99/21584 and U.S. Pat. No. 5,676,151).

When the cationic compound and nucleic acid molecules, such as DNA, are administered systemically, the ultrasound can be applied to one or several organs or tissues simultaneously to promote nucleic acid molecule delivery to multiple areas of the subject's body. Alternatively, the ultrasound can be applied selectively to specific areas or tissues to promote selective uptake of the nucleic acid molecules, such as DNA.

The transfection efficiency of the ultrasound can also be enhanced by using contrast reagents, which serve as artificial cavitation nuclei, such as Albunex (Molecular Biosystems, San Diego, Calif.), Imagent (Alliance Pharmaceutical, San Diego, Calif.), Levovist-SHU (Schering AG, Berlin, Germany), Definity (E.I. du Pont de Nemour, Wilmington, Del.), STUC (Washington University, St Louis, Mo.) and the introduction of gaseous microbubbles. A contrast reagent can be introduced locally, such as a joint; introduced systematically, with the enhancement of cavitation efficiency by focusing lithotripter shock waves at a defined area; or by targeting a contrast reagent to a particular site and then enhancing cavitation efficiency by focusing lithotripter shock waves.

(ii) Electroporation

Electroporation temporarily opens up pores in a cell's outer membrane by use of pulsed rotating electric fields. Methods and apparatus used for electroporation in vitro and in vivo are well known (see, e.g., U.S. Pat. Nos. 6,027,488, 5,993,434, 5,944,710, 5,507,724, 5,501,662, 5,389,069, 5,318,515). Standard protocols can be employed.

E. In Vitro and Ex Vivo Delivery of Nucleic Acids to Cells

1. In vitro Delivery

Nucleic acid molecules, such as DNA, can be added to synchronized cells in vitro either separately or mixed with one or more delivery agents and with or without the application of ultrasound or electrical energy. In general, if energy is applied, it is applied prior to contacting the cells with the nucleic acid molecule.

In general, nucleic acid molecules, such as DNA, mixed with cationic lipids/compounds can be added to synchronized cells as described in the EXAMPLES. Parameters important for optimization of the delivery of nucleic acid molecules, such as DNA, into target cells will be apparent to those of skill in this art. These parameters include, for example, the cell cycle phase during which nucleic acid molecule is added, the delivery method, the nucleic acid molecules, such as DNA, the concentration of nucleic acid molecules, the cell growth medium, the cell culture conditions, the length of time cells are exposed to the nucleic acid, the toxicity of the delivery method to the target cell type, and the amount and time of use of ultrasound or electroporation among other parameters. It can be necessary to optimize these parameters for different nucleic acid molecules, such as DNA, and target cell types. Such optimization is routine employing the guidance provided herein. In addition, the rapid screening method can provide direction as to what parameters may need to be adjusted to optimize delivery (see EXAMPLES). Alteration of culture conditions, time, reagent concentrations and other parameters, for use with different combinations of cationic compounds and target cell types and to optimize delivery, can be empirically determined. If ultrasound energy is required to be used to enhance transfection efficiency, it can be applied as described below and in the EXAMPLES. Electroporation can be performed as described below or by any suitable protocol known to those of skill in this art.

The contacting of cells with cationic compounds and nucleic acid molecules, such as DNA, in separate and distinct steps can be generally carried out as described in the EXAMPLES. Those of skill in the art can readily vary the order of the application of the components to the target cell based on the disclosure herein.

2. Ex vivo Gene Therapy

Delivery of nucleic acid molecules, such as DNA, is carried out as described above in in vitro delivery. After selection has been completed, cells harboring the nucleic acid molecules, such as DNA, are introduced into the subject target by a variety of means, including injection, such as subcutaneous, intramuscular, intraperitoneal, intravascular and intralymphatic and intra-articular injection. The cells can be administered with or without the aid of medical devices such as arthroscopes, other scopes or various types of catheters.

3. Gene Therapy in Connective Tissue and Rheumatic Diseases

Rheumatoid arthritis (RA) is a chronic inflammatory disease characterized by joint inflammation and progressive cartilage and bone destruction. Treatment of RA is problematic with current strategies since relatively high systemic doses are necessary to achieve therapeutic levels of anti-rheumatic drugs in the joints. In addition, the available treatments are associated with significant untoward side effects. Gene therapy is thus a more efficient system for delivery of therapeutic molecules to the site of inflammation in the treatment of connective tissue diseases, rheumatic diseases and chronic erosive joint diseases such as RA, osteoarthritis, ankylosing spondylitis and juvenile chronic arthritis.

In a diarthrodial movable joint, smooth articulation is ensured by the macromolecular structure of the articular cartilage which covers the ends of the bones. The cavity or joint space that occurs at the location of adjacent bones is lined by a tissue referred to as the synovium. The synovium contains macrophage-like type A cells (presumably derived from macrophage/monocyte precursors and exhibiting phagocytic activity) and fibroblast-like type B cells (more fibroblast in appearance and associated with production of hyaluronic acid and other components of the joint fluid). Underlying the synovium is a sparsely cellular subsynovium which can be fibrous, adipose or areolar in nature. Fibroblast-like synoviocytes (FLS) are distinguishable from normal fibroblast cells in the subintimal synovium by differential gene expression patterns. FLS have been shown to express high levels of uridine diphosphoglucose dehydrogenase (UDPGD), high levels of vascular cell adhesion molecule-1 (VCAM-1), intercellular adhesion molecule-1 (ICAM-1) as well as CD44 (hyaluronic acid receptor), fibronectin receptor and β3 integrins. Sublining fibroblasts or fibroblasts from other sources do not express these markers or express them at lower levels [see, e.g., Edwards (1995) Ann. Rheum. Dis. 54:395-397; Firestein (1996) Arthritis Rheum. 39:1781-1790; Edwards (2000) Arthritis Res. 2:344-347].

Disease progression in RA involves the thickening of the synovial lining due to the proliferation of fibroblast-like synoviocytes (FLS) and infiltration by inflammatory cells (e.g., lymphocytes, macrophages and mast cells). The normal biology of synoviocytes is also altered in the pathological process of RA, including invasion and destruction of articular cartilage and bone. In addition to the production of elastase and collagenase, synoviocytes mediate the pathophysiological process of RA by expression of cell surface proteins involved in the recruitment and activation of lymphocytes and macrophages within the synovium. Proliferation of synovial cells leads to a pannus tissue that invades and overgrows cartilage, leading to bone destruction and destruction of joint structure and function. Proinflammatory cytokines, for example, tumor necrosis factor-á (TNF-á) and interleukin-1 (IL-1) play key roles in inflammation and joint damage associated with RA. Pathological effects caused by these cytokines include leukocytic infiltration leading to synovial hyperplasia, cell activation, cartilage breakdown and inhibition of cartilage matrix synthesis.

Nucleic acid transfer to rheumatoid synovial tissue may result in the production of mediators that inhibit inflammation or hyperplasia or provide toxic substances that specifically destroy the diseased synovium. Retroviral delivery of nucleic acid encoding interleukin-1 receptor antagonist (IL1-RA) ex vivo and transduction of synoviocytes has been used in gene therapy of RA in humans to inhibit inflammation [see, e.g., Evans (1996) Human Gen. Ther. 7:1261-1280 and Del Vecchio et al. (2001) Arthritis Res. 3:259-263]. Adenoviral vectors have been proposed for delivery of nucleic acid encoding an IL-1 receptor antagonist to synoviocytes in in vivo transduction methods [see, e.g., U.S. Pat. No. 5,747,072 and PCT Application Publication No. WO 00/52186].

Artificial chromosomes provide advantages over virus-based systems for gene therapy. For example, artificial chromosome expression systems (ACes), and other artificial chromosomes as described in U.S. Pat. Nos. 6,025,155 and 6,077,697 and PCT Application No. W097/401 83, serve as non-integrating, non-viral vectors with a large capacity for delivering large nucleic acids and/or multiple copies of a particular nucleotide sequence into cells, such as synoviocytes, both in vitro and in vivo. Such artificial chromosome systems offer further advantages in that they allow stable and predictable expression of genes producing single or multiple proteins over long periods of time.

The methods provided herein can be used to introduce large nucleic acids, such as, for example, artificial chromosomes, into primary cells, such as, for example, synoviocytes (e.g., fibroblast-like synoviocytes) and skin fibroblasts, and skeletal muscle fibroblast cell lines. Thus, included among the methods provided herein is a method for introducing heterologous nucleic acid into a synoviocyte by introducing in a chromosome, such as for example an artificial chromosome, into the synoviocyte. In one such embodiment, the artificial chromosome is an ACes. The synoviocyte can be, for example, a fibroblast-like synoviocyte.

A particular method provided herein for introducing a large nucleic acid molecule into a synoviocyte includes steps of obtaining a synoviocyte that is in a pre-selected phase and contacting the synoviocyte with the nucleic acid molecule. In one method, the nucleic acid molecule is contacted with a synchronous population of synoviocytes in which the population has been synchronized to be in a pre-selected phase at the time of contact.

Such methods can further include steps of exposing the nucleic acid molecule to a delivery agent and contacting the synoviocyte with the nucleic acid molecule. In a particular embodiment of this method, the delivery agent is not energy. In one embodiment, the large nucleic acid molecule is a chromosome. For example, the nucleic acid can be an artificial chromosome, such as an ACes. In a particular embodiment, the synoviocyte is a fibroblast-like synoviocyte. Any delivery agents, such as described herein, can be used in such methods. For example, the delivery agent can be one that includes a cationic compound.

Also provided are methods for introducing a large nucleic acid molecule into a synoviocyte that include a step of obtaining a synoviocyte that is in a pre-selected phase and contacting the synoviocyte with the nucleic acid molecule. Such methods can further include steps of exposing the nucleic acid molecule to a delivery agent, exposing the synoviocyte to a delivery agent and contacting the synoviocyte with the nucleic acid molecule, whereby the nucleic acid molecule is delivered into the synoviocyte, and wherein the steps are performed sequentially in any order or simultaneously. In some embodiments of the method, if the delivery agent is energy, it is not applied to the nucleic acid molecule and it is not applied to the synoviocyte after contacting the synoviocyte with the nucleic acid molecule. The nucleic acid can be any nucleic acid. In particular embodiments, the nucleic acid is a large nucleic acid, chromosome, artificial chromosome or ACes. In a further particular embodiment, the synoviocyte is a fibroblast-like synoviocyte. Delivery agents, such as described herein, can be used in such methods. For example, the delivery agent can be one that includes a cationic compound.

Another method for delivering a nucleic acid molecule into a synoviocyte provided herein includes steps of contacting the synoviocyte in the presence or absence of the nucleic acid molecule with a delivery agent, and applying ultrasound energy or electrical energy to the synoviocyte, wherein the contacting and applying are performed sequentially or simultaneously, and then contacting the synoviocyte with the nucleic acid molecule, whereby the nucleic acid molecule is delivered into the synoviocyte. The nucleic acid can be any nucleic acid. In particular embodiments, the nucleic acid is a large nucleic acid, chromosome, artificial chromosome or ACes. In a further particular embodiment, the synoviocyte is a fibroblast-like synoviocyte. Numerous delivery agents, including agents such as those described herein, can be used in such methods. For example, the delivery agent can be one that includes a cationic compound. In one embodiment, the energy is ultrasound.

Thus, provided herein are methods of delivering nucleic acids, in particular, large nucleic acids, such as chromosomes, including artificial chromosomes, e.g., ACes, into primary cells, including synoviocytes and fibroblasts. These methods can be used in vitro and in vivo.

Also provided herein is a synoviocyte in a pre-selected phase comprising a large heterologous nucleic acid, a heterologous chromosome or portion thereof, or an artificial chromosome. In one embodiment, the artificial chromosome is an ACes. Such synoviocytes include fibroblast-like synoviocytes. The synoviocytes can be from any species, including, but not limited to mammalian species. For example, synoviocytes containing large nucleic acids, such as for example artificial chromosomes (e.g., ACes) include primate synoviocytes, as well as rodent, rabbit, monkey, dog, horse and human synoviocytes.

The ability to achieve delivery of large nucleic acids into such cells demonstrates the usefulness of the methods in gene therapy applications as well as in the testing in animal models of disease of possible therapeutic molecules for use in gene therapy methods. Thus, provided herein are methods of treating diseases or modulating disease processes which include steps of introducing a large nucleic acid molecule, chromosome or portion thereof, or artificial chromosome into a subject who has the disease.

A method for treating or modulating a rheumatic disease process in a subject is provided herein. In one embodiment, the method includes steps of introducing a large nucleic acid into the subject, wherein the large nucleic acid contains nucleic acid that is or that encodes an agent that modulates a rheumatic disease process. For example, the nucleic acid can be or can encode a molecule that has an anti-rheumatic effect. Processes associated with rheumatic diseases are known in the art and are described herein. For example, one such process is an inflammatory process that includes processes of cell activation, infiltration, proliferation and recruitment. In a particular embodiment of this method, the disease is rheumatoid arthritis. The nucleic acid can be, for example, a chromosome or portion thereof or an artificial chromosome, e.g., an ACes. In particular embodiments, the large nucleic acid is introduced into a site of inflammation in the subject. One possible site of inflammation is a joint.

Also provided is a method for treating a rheumatic disease in a subject in which a large nucleic acid is introduced into the subject, wherein the large nucleic acid contains nucleic acid that is or that encodes a therapeutic agent. For example, the nucleic acid can be or can encode a molecule that has an anti-rheumatic effect. In a particular embodiment of this method, the disease is rheumatoid arthritis. The nucleic acid can be, for example, a chromosome or portion thereof or an artificial chromosome, e.g., an ACes. In an embodiment of this method, the large nucleic acid is introduced into a site of inflammation in the subject. One possible site of inflammation is a joint.

In the methods for modulating a rheumatic disease process or treating a rheumatic disease, the method can be practiced in any format, including ex vivo and in vivo formats. Thus, for example, the nucleic acid can be introduced into a cell in vitro and then transferred into the subject. Alternatively, the nucleic acid can be introduced into a cell in vivo. In a particular embodiment, the nucleic acid is introduced into a synoviocyte, which can be, for example, a fibroblast-like synoviocyte. The nucleic acid that is introduced can comprise any nucleic acid that is or that encodes a molecule that has an anti-rheumatic effect in the subject. For example, the molecule may alter, counteract or diminish a process of the disease. The molecule may ameliorate symptoms of the disease. Molecules that provide anti-rheumatic effects in subjects with RA are known in the art [see, e.g., Vervoordeldonk and Tak (2001) Best Prac. Res. Clin. Rheumatol. 15:771-788 and WO 00/52186]. Such molecules include anti-inflammatory or immunomodulatory molecules. For example, interleukin-1 receptor antagonists, soluble interleukin-1 receptor, soluble tumor necrosis factor receptor, interferon-β, interleukin-4, interleukin-10, interleukin-13, transforming growth factor β, dominant negative IkappaB-kinase, FasL, Fas-associated death domain protein or CTLA-4 are among molecules that can have anti-rheumatic effects.

Also provided is a method of identifying, evaluating or testing a nucleic acid as a potential therapeutic agent in the treatment of a connective tissue or rheumatic disease by introducing into an animal model of a connective tissue or rheumatic disease a large nucleic acid molecule. The nucleic acid molecule can be one that includes nucleic acid that is or encodes a candidate therapeutic agent. The method may include a step of determining if the nucleic acid molecule has any effects, and in particular any anti-rheumatic effects, on the animal. For example, in determining if the nucleic acid molecule has any effects on the animal, it can be evaluated whether one or more conditions of the disease is effected, such as, for example, amelioration of or reduction in an adverse condition. In a particular embodiment, the disease is a rheumatic disease, such as, for example rheumatoid arthritis. The animal is any animal in which the disease can be modeled. For example, the animal can be a mammal. In particular embodiments the animal is a monkey, rodent, rabbit, dog, cat, horse, cow, pig or primate. The large nucleic acid can be, for example, a chromosome, or portion thereof, or an artificial chromosome, for example, an ACes. In particular embodiments, the nucleic acid molecule is in a synoviocyte, such as, for example, a fibroblast-like synoviocyte. In further embodiments, the nucleic acid is introduced into a joint of the animal. The nucleic acid molecule can be introduced into the animal using in vitro or in vivo formats. For example, the nucleic acid can be introduced into a cell in vitro and then be transferred into the animal. In another embodiment, the nucleic acid is introduced into a cell in vivo.

Animal models include, for example, animal models of RA. Several animal models of RA, and methods for generating such models, are known in the art. Such models include adjuvant-induced arthritis (AA) [see, e.g., Kong et al. (1999) Nature 4023:304-309] and collagen type II-induced arthritis [see, e.g., Tak et al. (1 999) Rheumatology 38:362-369; Han et al. (1998) Autoimmunity 28:197-208; Gerlag et al. (2000) J. Immunology 165:1652-1658]. For example, experimental induction of adjuvant-induced arthritis in Lewis rats leads to severe inflammation in the bone marrow and soft tissues surrounding joints accompanied by extensive local bone and cartilage destruction, loss of bone mineral density and crippling [see, e.g., Bendele et al. (1 999) Arthritis Rheum. 42:498-506].

The following examples are included for illustrative purposes only and are not intended to limit the scope of the invention.

### EXAMPLE 1

Preparation of Artificial Chromosomes

A. GFP Chromosome Contained in A9 Cell Line

Plasmids

Plasmid pIRES-EGFP (see SEQ ID No. 13, plasmid obtained from Clontech, Calif., and is well known, see, e.g., U.S. Pat. Nos. 6,034,228, 6,037,133, 5,985,577, 5,976,849, 5,965,396, 5,976,796, 5,843,884, 5,962,265, 5,965,396; see, also, U.S. Pat. No. 4,937,190). This plasmid contains the internal ribosome entry site (IRES; Jackson (1990) Trends Biochem. 15:477-483; Jang et al. (1988) J. Virol. 62:2636-2643) of the encephalomyocarditis virus (ECMV) between the MCS and the enhanced green fluorescent protein (EGFP) coding region. This permits the gene of interest (cloned into the MCS) and the EGFP gene to be translated from a single bicistronic mRNA transcript. Plasmid pIRES2-EGFP is designed for selection, by flow cytometry and other methods, of transiently transfected mammalian cells that express EGFP and the protein of interest. This vector can also be used to express EGFP alone or to obtain stably transfected cell lines without drug and clonal selection.

Enhanced GFP (EGFP) is a mutant of GFP with a 35-fold increase in fluorescence. This variant has mutations of Ser to Thr at amino acid 65 and Phe to Leu at position 64 and is encoded by a gene with optimized human codons (see, e.g., U.S. Pat. No. 6,054,312). EGFP is a red-shifted variant of wild-type GFP (Yang et al. (1996) Nucl. Acids Res. 24:4592-4593; Haas et al. (1996) Curr. Biol. 6:315-324; Jackson et al. (1990) Trends Biochem. 15:477-483) that has been optimized for brighter fluorescence and higher expression in mammalian cells (excitation maximum=488 nm; emission maximum=507 nm). EGFP encodes the GFPmut1 variant (Jackson (1990) Trends Biochem. 15:477-483) which contains the double-amino-acid substitution of Phe-64 to Leu and Ser-65 to Thr. The coding sequence of the EGFP gene contains more than 190 silent base changes which correspond to human codon-usage preferences (Jang et al. (1988) J. Virol. 62:2636-2643). Sequences flanking EGFP have been converted to a Kozak consensus translation initiation site (Huang et al. (1990) Nucleic Acids Res. 18: 937-947) to further increase the translation efficiency in eukaryotic cells.

Plasmid pIRES-EGFP was dervied from PIRESneo (orignally called pCIN4) by replacing the neo gene downstream of the IRES sequence with the EGFP coding region. The IRES sequence permits translation of two open reading frames from one mRNA transcript. The expression cassette of pIRES-EGFP contains the human cytomegalovirus (CMV) major immediate early promoter/enhancer followed by a multiple cloning site (MCS), a synthetic intron (IVS; Huang et al. (1990) Nucleic Acids Res. 18: 937-947), the EMCV IRES followed by the EGFP coding region and the polyadenylation signal of bovine growth hormone.

Location of Features (with reference to SEQ ID No. 13):

Human cytomegalovirus (CMV) immediate early promoter: 232-820;

MCS 909-974;

IVS 974-1269;

IRES of ECMV 1299-1884;

Enhanced green fluorescent protein (EGFP) gene 1905-2621;

fragment containing the bovine polyA signal 2636-2913;

Col E1 origin of replication 3343-4016; and

Ampicillin resistance gene 5026-4168

Propagation in E. coli

Suitable host strains: DH5a, HB101, and other general purpose strains. Single-stranded DNA production requires a host containing an F plasmid such as JM101 or XL1-Blue.

Selectable marker: plasmid confers resistance to kanamycin (30 μg/ml) to E. coli hosts.

E. coli replication origin: pUC

Copy number: ˜500

Plasmid incompatibility group: pMB1/ColE1

pCHEGFP2

Plasmid pCHEGFP2 was constructed by deletion of the Nsi1/SmaI fragment from pIRES-EGFP. Plasmid pIRES-EGFP contains the coding sequence for a 2.1 kB Nru 1/Xho fragment of pCHEGFP2 containing the CMV promoter, synthetic intron, EGFP coding sequence and bovine growth hormone polyadenylation signal. Digestion of pIRES-EGFP with Nru 1 and Sma 1, yielded a 2.1 kb fragment. Digested DNA was fractionated by agarose gel electrophoresis, the separated band was excised and then eluted from the gel using the Qiaex 11 gel purification system (Qiagen, Mississauga, Ontario).

pFK161

Cosmid pFK161 was obtained from Dr. Gyula Hadlaczky and contains a 9 kb NotI insert derived from a murine rDNA repeat (see clone 161 described in PCT Application Publication No. WO97/40183 by Hadlaczky et al. for a description of this cosmid). This cosmid, referred to as clone 161 contains sequence corresponding to nucleotides 10,232-15,000 in SEQ ID NO. 16. It was produced by inserting fragments of the megachromosome (see, U.S. Pat. No. 6,077,697 and International PCT application No. (WO 97/40183); for example, H1D3, which was deposited at the European Collection of Animal Cell Culture (ECACC) under Accession No. 96040929, is a mouse-hamster hybrid cell line carrying this megachromosome) into plasmid pWE15 (Stratagene, La Jolla, Calif.) as follows. Half of a 100 μl low melting point agarose block (mega-plug) containing isolated SATACs was digested with NotI overnight at 37° C. Plasmid pWE15 was similarly digested with NotI overnight. The mega-plug was then melted and mixed with the digested plasmid, ligation buffer and T4 ligase. Ligation was conducted at 16° C. overnight. Bacterial DH5α cells were transformed with the ligation product and transformed cells were plated onto LB/Amp plates. Fifteen to twenty colonies were grown on each plate for a total of 1 89 colonies. Plasmid DNA was isolated from colonies that survived growth on LB/Amp medium and was analyzed by Southern blot hybridization for the presence of DNA that hybridized to a pUC19 probe. This screening methodology assured that all clones, even clones lacking an insert but yet containing the pWE15 plasmid, would be detected.

Liquid cultures of all 189 transformants were used to generate cosmid minipreps for analysis of restriction sites within the insert DNA. Six of the original 189 cosmid clones contained an insert. These clones were designated as follows: 28 (˜9-kb insert), 30 (˜9-kb insert), 60 (˜4-kb insert), 113 (˜9-kb insert), 157 (˜9-kb insert) and 161 (˜9-kb insert). Restriction enzyme analysis indicated that three of the clones (113, 157 and 161) contained the same insert.

For sequence analysis the insert of cosmid clone no. 1 61 was subcloned as follows. To obtain the end fragments of the insert of clone no. 161, the clone was digested with NotI and BamHI and ligated with NotI/BamHI-digested pBluescript KS (Stratagene, La Jolla, Calif.). Two fragments of the insert of clone no. 161 were obtained: a 0.2-kb and a 0.7-kb insert fragment. To subclone the internal fragment of the insert of clone no. 161, the same digest was ligated with BamHI-digested pUC19. Three fragments of the insert of clone no. 161 were obtained: a 0.6-kb, a 1.8-kb and a 4.8-kb insert fragment.

The insert corresponds to an internal section of the mouse ribosomal RNA gene (rDNA) repeat unit between positions 7551-15670 as set forth in GENBANK accession no. X82564, which is provided as SEQ ID NO. 5. The sequence data obtained for the insert of clone no. 161 is set forth in SEQ ID NOS. 6-12. Specifically, the individual subclones corresponded to the following positions in GENBANK accession no. X82564 (i.e., SEQ ID NO. 5) and in SEQ ID NOs. 6-12:

| | in X82564 | | | |
|---|---|---|---|---|
| Subclone | Start | End | Site | SEQ ID No. |
| 161k1 | 7579 | 7755 | NotI, BamHI | 6 |
| 161m5 | 7756 | 8494 | BamHI | 7 |
| 161m7 | 8495 | 10231 | BamHI | 8 (shows only sequence |
| | | | | corresponding to nt. |
| | | | | 8495-8950), |
| | | | | 9 (shows only sequence |
| | | | | corresponding to nt. |
| | | | | 9851-10231) |
| 161m12 | 10232 | 15000 | BamHI | 10 (shows only sequence |
| | | | | corresponding to nt. |
| | | | | 10232-10600), |
| | | | | 11 (shows only sequence |
| | | | | corresponding to nt. |
| | | | | 14267-15000) |
| 161k2 | 15001 | 15676 | NotI, BamHI | 12 |

The sequence set forth in SEQ ID NOs. 6-12 diverges in some positions from the sequence presented in positions 7551-15670 of GENBANK accession no. X82564. Such divergence can be attributable to random mutations between repeat units of rDNA.

For use herein, the rDNA insert from the clone was prepared by digesting the cosmid with NotI and BglII and was purified as described above. Growth and maintenance of bacterial stocks and purification of plasmids were performed using standard well known methods (see, e.g., Sambrook et al. (1989) Molecular Cloning: A Laboratory Manual, 2nd Edition, Cold Spring Harbor Laboratory Press), and plasmids were purified from bacterial cultures using Midi- and Maxi-preps Kits (Qiagen, Mississauga, Ontario).

B. Preparation of the GFP, Murine A9 Cell Line

Cell Culture and Transfection

The murine A9 cell line was obtained from ATCC and cells were thawed and maintained as described below. Briefly, cells were plated at a density of 2×10⁶ cells per 15 cm tissue culture dish (Falcon, Becton Dickinson Labware, Franklin Lakes, N.J.) in growth medium containing of 90% DMEM (Canadian Life Technologies Burlington, ON) and 10% FBS (Can Sera, Rexdale ON), and were maintained at 37° C., 5% CO₂. Cultures were routinely passaged when cells reached 70%-80% confluence. Sub culturing was carried out as follows: medium was removed by aspiration, 10 ml of 1× trypsin-EDTA (Canadian Life Technologies Burlington, ON) was dispensed onto the cell monolayer and the dish gently swirled to distribute the trypsin-EDTA. Finally, the bulk of the trypsin-EDTA was removed by aspiration, and the dish placed at 37° C. for 5 minutes. To quench the trypsin-EDTA, 10 ml of growth medium was added to the dish, and the single cell suspension was transferred to a 50 ml conical tube. Cell counts were performed using a cell counting apparatus (Beckman-Coulter, Hialeah Fla.). The cells were diluted and re-plated as described above. For cryo-storage, cultures were harvested by treatment with trypsin-EDTA, counted and the cell suspension then centrifuged at 500×g for 5 minutes in a swinging bucket centrifuge. The cell pellet was resuspended in freezing medium containing 90% DMEM, 20% FBS and 10% DMSO (Sigma-Aldrich, Oakville, ON) at a density of 1×10⁷ cells/ml. One ml aliquots of the cell suspension were then dispensed into cryo-vials (NUNC, Rochester N.Y.), frozen over night in an isopropanol filled container (NUNC, Rochester N.Y.) and placed at −70° C. and then transferred to the gas phase of a liquid nitrogen freezer for long-term storage.

A9 cells were transfected using the Ca₂PO₄ co-precipitation method (see, e.g., Graham et al. (1978) Virology 52:456-457; Wigler et al. (1979) Proc. Natl. Acad. Sci. U.S.A. 76:1373-1376; and (1990) Current Protocols in Molecular Biology, Vol. 1, Wiley Inter-Science, Supplement 14, Unit 9.1.1-9.1.9). One day prior to transfection, A9 cells were plated at a density of 2×10⁶ cells per 10 cm dish and 3 hours before transfection the medium was replaced with fresh growth medium. 140 μg of the 9 kb rDNA, NotI and 5 μg of the 2.1 kB CMV-EGFP XhoI/NruI fragments were mixed, co-precipitated and used to prepare the Ca₂PO₄ co-precipitate (Calcium Phosphate Transfection System, (Canadian Life Technologies Burlington, ON) which was distributed onto 2 10-cm dishes of subconfluent A9 cells. The DNA-Ca₂PO₄ complexes were left on the cells for 18 hours, after which the precipitate was removed by aspiration and cells were subjected to glycerol shock for 1.5 minutes. After glycerol shock, the cell monolayers were gently washed with 2×10 ml of dPBS (Canadian Life Technologies Burlington, ON), followed by addition of 10 ml pre-warmed growth medium. Finally dishes were returned to the incubator and were maintained at 37° C., 5% CO₂. After 3 hours recovery, each dish was passaged onto 3×15 cm tissue dishes

GFP fluorescence of cultures was monitored visually during culture using an inverted microscope equipped with epifluorescence illumination (Axiovert 25; Zeiss, (North York ON) and #41017 Endow GFP filter set (Chroma Technologies, Brattleboro, Vt.). Enrichment of GFP expressing populations was carried out as described below.

Enrichment of GFP Expressing Cell Populations by Fluorescence Activated Cell Sorting

Cell sorting was carried out using a FACS Vantage flow cytometer (Becton Dickinson Immunocytometry Systems, San Jose, Calif.) equipped with turbo-sort option and 2 Innova 306 lasers (Coherent, Palo Alto Calif.). For cell sorting a 70 μm nozzle was used. The sheath buffer was changed to PBS (maintained at 20 p.s.i.). GFP was excited with a 488 nm Laser beam and excitation detected in FL1 using a 500 EFLP filter. Forward and side scattering was adjusted to select for viable cells. Only viable cells were then analyzed for GFP fluorescence. Gating parameters were adjusted using wild type A9 cells as negative control and GFP CHO cells as positive control.

For the first round of sorting, A9 cells were harvested 4 days post-transfection, resuspended in 10 ml of growth medium and sorted for GFP expressing populations using parameters described above. GFP positive cells were dispensed into a volume of 5-10 ml of growth medium supplemented with 1× penicillin/streptomycin (Canadian Life Technologies Burlington, ON) while non-expressing cells were directed to waste. The expressing cells were further diluted to 50 ml using the same medium, plated onto 2×15 cm dishes and cultured as described in the previous section. When the sorted populations reached confluence they were re-sorted to enrich for GFP expressing cells. A total of 4 sequential sorts were carried out, achieving enrichments of as high as 89% GFP expressing cells after the final sort. The final GFP expressing populations were expanded for cryo-preservation and for fluorescence in-situ hybridization screening (see below). Single cell clones were established from populations of interest by using the flow cytometer to direct GFP expressing single cells to individual wells of 96 well plates. These were cultured as described above.

Fluorescence In-Situ Hybridization

Fluorescence In-Situ Hybridization (FISH) screening was carried out on GFP enriched populations and single cell clones to detect amplification and/or artificial chromosome formation. Preparation of metaphase spreads and hybridizations were performed (see, Telenius et al. (1999) Chromosome Res 7:3-7). Probes used include pSAT 1, which recognizes the mouse major repeat (see, e.g., Wong et al. (1988) Nucl. Acids Res. 16:11645-11661), pFK161, which hybridizes to the mouse rDNA-containing regions and a PCR generated probe against the mouse minor repeat.

Thus, in one method provided herein for generating an artificial chromosome, such as an ACes, heterologous nucleic acid that includes a selectable marker, e.g., nucleic acid encoding a fluorescent protein or other protein that can be readily detected using flow cytometry-based methods or other methods, including, for example, fluorimetry, cell imaging or fluorescence spectroscopy, is introduced into a cell. For example, rDNA and DNA encoding enhanced green fluorescent protein (EGFP) can be introduced into cells, e.g., A9 cells. The transfected cells can be selected on the basis of properties detectable by flow cytometry-based methods, or other methods, including, for example, fluorimetry, cell imaging or fluorescence spectroscopy, e.g., fluorescent properties. For example, cells containing a fluorescent protein can be isolated from nontransfected cells using a fluorescence-activated cell sorter (FACS). If the sorting is conducted prior to chromosomal analysis of the cells for the presence of artificial chromosomes, it provides a population of transfected cells that can be enriched for artificial chromosomes and thus facilitates any subsequent chromosomal analysis of the cells and identification and selection of cells containing an artificial chromosome, e.g., ACes. For example, the cells can be analyzed for indications of amplification of chromosomal segments, the presence of structures that may arise in connection with amplification and de novo artificial chromosome formation and/or the presence of artificial chromosomes, such as ACes. Analysis of the cells typically involves methods of visualizing chromosome structure, including, but not limited to, G- and C-banding and FISH analyses using techniques described herein and/or known to those of skill in the art. Such analyses can employ specific labelling of particular nucleic acids, such as satellite DNA sequences, heterochromatin, rDNA sequences and heterologous nucleic acid sequences, that can be subject to amplification. During analysis of transfected cells, a change in chromosome number and/or the appearance of distinctive, for example, by increased segmentation arising from amplification of repeat units, chromosomal structures will also assist in identification of cells containing artificial chromosomes.

C. Purification of Artificial Chromosomes by Flow Cytometry and Preparation of DNA from Flow Sorted Chromosomes

Artificial chromosomes were purified from the host cell by flow cytometry (see de Jong (1999) Cytometry 35:129-133). Briefly, purification was performed on FACS Vantage flow cytometer (Becton Dickinson Immunocytometry Systems, San Jose, Calif.) equipped with a Trubo-Sort Option and two Innova 306 lasers (Coherent, Palo Alto, Calif.). The Turbo Sort Option modification include increasing the maximum system pressure from 20 lb/in² to 60 lb/in², increasing the drop drive frequency from 50,000 drops/s to a maximum of 99,000 drops/s and increasing the deflection plate voltages from a maximum 6,000 V to 8,000 V. Other modifications are made to the instrument to accommodate the higher pressures. Hoechst 35258 was excited with the primary UV laser beam, and excitation detected in FLI by using 420 nm hand-pass filter. Chromomycin A3 was excited by the second laser set at 458 nm and fluorescence detected in FL 4 by using a 475 nm long-pass filter. Both lasers had an output of 200 mW. Bivariate distributions (1,024×1024 channels) were accumulated during each sort. For all chromosome sorts, the sheath pressure was set at 30 lb/in² and a 50 μm diameter nozzle was installed. A drop delay profile was performed every morning and repeated after any major plug. Alignment of the instrument was performed daily by using 3.0 μm diameter Sphero rainbow beads (Spherotech, Libertyville, Ill.). Alignment was considered optimized when a CV of 2.0% or less was achieved for FL1 and FL4.

Condensing agents (hexylene glycol, spermine and spermidine) were added to the sheath buffer to maintain condensed chromosomes after sorting. The sheath buffer contains 15 nM Tris HCl, 0.1 mM EDTA, 20 mM NaCl, 1 % hexylene glycol, 100 mM glycine, 20 μM spermine and 50 μM spermidine. The sorted chromosomes were collected in 1.5 ml screw-capped Eppendorf tubes at 4° C. at a concentration of approximately 1×10⁶ chromosomes/ml, which were then stored at 4° C.

For preparation of purified genomic DNA, sorted chromosome samples were brought to 0.5% SDS, 50 mm EDTA and 100 μg/ml Proteinase K, then incubated for 18 hours at 50° C. 1 μl of a 20 mg/ml glycogen solution (Boehringer Mannheim) was added to each sample, followed by extraction with an equal volume of Phenol: Chloroform: Isoamyl Alcohol (25:24:1). After centrifugation at 21,000×g for 10 min, the aqueous phases were transferred to fresh microfuge tubes and were re-extracted as above. 0.2 volumes of 10 M NH₄OAC, 1 μl of 20 mg/ml glycogen and 1 volume of iso-propanol were added to the twice extracted aqueous phases which were then vortexed and centrifuged for 15 minutes at 30,000×g (at room temperature). Pellets were washed with 200 μl of 70% ethanol and re-centrifuged as above. The washed pellets were air-dried then resuspended in 5 mM Tris-Cl, pH 8.0 at 0.5-2×10⁶ chromosome equivalents/μl.

PCR was carried out on DNA prepared from sorted chromosome samples essentially as described (see, Co et al. (2000) Chromosome Research 8:183-191) using primers sets specific for EGFP and RAPSYN. Briefly, 50 μl PCR reactions were carried out on genomic DNA equivalent to 10,000 or 1000 chromosomes in a solution containing 10 mM Tris-Cl, pH 8.3, 50 mM KCl, 200 μM dNTPs, 500 nM of forward and reverse primers, 1.5 mM MgCl₂, 1.25 units Taq polymerase (Ampli-Taq, Perkin-Elmer Cetus, Calif.). Separate reactions were carried out for each primer set. The reaction conditions were as follows: one cycle of 10 min. at 95° C., then 35 cycles of 1 min. at 94° C., 1 min. at 55° C., 1 min at 72° C. and finally one cycle of 10 min at 72° C. After completion the samples were held at 4° C. until analyzed by agarose gel electrophoresis using the following primers (SEQ ID Nos. 1-4, respectively):

| | |
|---|---|
| EGFP | 5′-cgtccaggagcgcaccatcttctt-3′; |
| forward | |
| primer | |
| EGFP | 3′-atcgcgcttctcgttggggtcttt-3′; |
| reverse | |
| primer | |
| RAPSYN | 5′-aggactgggtggcttccaactcccagacac-3′; and |
| forward | |
| primer | |
| RAPSYN | 5′-agcttctcattgctgcgcgccaggttcagg-3′. |
| reverse | |
| primer | |

All primers were obtained from Canadian Life Technologies, Burlington, ON.

### EXAMPLE 2

Preparation of Cationic Vesicles

Vesicles were prepared at a lipid concentration of 700 nmoles/ml lipid (cationic lipid/DOPE 1:1) as follows. In a glass tube (10 ml) 350 nmol cationic lipid (SAINT-2) was mixed with 350 nmol dioleoylphosphatidylethanolamine (DOPE), both solubilized in an organic solvent (Chloroform, Methanol or Chloroform/Methanol 1:1, v/v). Dioleoylphosphatidylethanolamine (DOPE; Avanti Polar Lipids, Alabaster, AL) forms inverse hexagonal phases in a membrane and weakens the membrane. Other effectors that can be used are cis-unsaturated phosphatidylethanolamines, cis-unsaturated fatty acids and cholesterol. Cis-unsaturated phosphatidylcholines are less effective.

The solvent was evaporated under a stream of nitrogen (15 min/250 μl solvent at room temperature). The remaining solvent was removed totally by drying the lipid for 15 min in an desiccator under high vacuum from a vacuum pump. To the dried mixture was added 1 ml ultrapure water. This was vortexed vigorously for about 5 min. The resulting solution was sonicated in an ultrasonication bath (Laboratory Supplies Inc. NY) until a clear solution was obtained. The resulting suspension contained a population of unilamellar vesicles with a size distribution between 50 to 100 nm.

### EXAMPLE 3

Preparation of Cationic Vesicles via Alcoholic Injection

In a glass tube (10 ml) 350 nmol cationic lipid (Saint-2) was mixed with 350 nmol DOPE, both solubilized in an organic solvent (chloroform, methanol or chloroform/methanol 1/1). The solvent was evaporated under a stream of nitrogen (15 min/250 μl solvent at room temperature). The remaining solvent was removed totally by drying the lipid for 15 min under high vacuum. This was then reconstituted in 100 μl pure ethanol.

### EXAMPLE 4

Transfection of beta ACes into V79-4 Cell Line

Transfection Procedure for Various Transfection Agents

All compounds were tested in a Chinese Hamster lung fibroblast line (V79-4, ATCC number CCL-39). Approximately 17 hours (2 cell doublings) prior to transfection, exponentially growing cells were trypsinized and plated at 250,000 cells per well into a 6 well petri dish with Dulbecco's Modified Eagle Medium (Life Technologies, Burlington, ON) and supplemented with 10% FBS (Can Sera, Rexdale ON)). At the time of transfection, the number of cells per well was estimated to be approximately 1 million. For transfection, each individual manufacturer's protocol for complexing to naked DNA was followed, with the exception that the amount of transfection agent used was varied, to reflect the different amount and type of DNA present, as well as the different ionic strength of the complexing. One million ACes (in a volume of 800 μl) were typically combined with the transfection agent in a wide range of concentrations (between 5 times and 100 times the lowest manufacturers suggested concentration). The ACes/transfection mixture was allowed to complex for the time recommended by the manufacturer, in volumes ranging from 0.8 ml to 1.9 ml; some manufacturers recommend adding media to the complexing reaction. The complexed mixture was then applied to the recipient cells and transfection allowed to proceed according to the manufacturer's protocol. Details on the various conditions used with different agents are presented in Table 1.

Transfection Procedure for Superfect Agent

Superfect was tested in a Chinese Hamster lung fibroblast line (V79-4, ATCC number CCL-39). Approximately 17 hours (2 cell doublings) prior to transfection; exponentially growing cells were trypsinized and plated at 250,000 cells per well into a 6 well petri dish with Dulbecco's Modified Eagle Medium (Life Technologies, Burlington, ON) and supplemented with 10% FBS (Can Sera, Rexdale ON). One million ACes in 800 μl of sort buffer was complexed to 10 μl of Superfect reagent. Complex was incubated at room temperature for 10 minutes. At the time of transfection, the number of cells per well was estimated to be approximately 1 million. Media was removed from wells and 600 μl of DMEM and 10% FBS was added. Superfect:ACes complex was added to the wells drop-wise and allowed to incubate for 3 hours at 37° C. After incubation, transfected cells were trypsinized and transferred to 15 cm dishes with 25 ml DMEM and 10% FBS and allowed to attach for 24 hours. After 24 hours, selection medium containing of 0.7 mg/ml hygromycin B was added to each well. The selection medium was changed every 2-3 days. After 10-12 days colonies were screened for Beta-galactosidase expression and/or FISHed for detection of intact chromosome.

Example of Application of the Determination of the Chromos Index

Approximately 1×10⁶ V79-4 cells were transfected with 1×10⁶ IdUrd-labeled ACes complexed with a delivery agent (i.e., Lipofectamine PLUS and Lipofectamine or Superfect). The transfected cells were then fixed in ethanol. Fixed cells were denatured and exposed to FITC-conjugated antibody that specifically binds to BrdU/IdUrd-labeled nucleic acids.

The percentage of transfected cells containing IdUrd-labeled ACes was determined using flow cytometry and collecting FITC fluorescence. Data were accumulated to form bivariate channel distribution showing forward scatter versus green fluorescence (IdUrd-FITC). The fluorescence level at which cells were determined to be positive was established by visual inspection of the histogram of negative control cells such that the gate for the negative cells was set such that 1% appeared in the positive region.

The number of cells recovered at 24 hours post-transfection was determined by counting an aliquot using a Coulter Counter. To determine the control plating efficiency of a recipient cell line, the untreated cells were plated at 600-1000 cells per 10 cm petri dish in growth medium and left stationary in a 5% CO₂ incubator at 37° C. for approximately five cell cycles or until average colony was made up of 50 cells. At this point the number of viable colonies was determined. The treated cells were seeded at 1000 cells if the CPE is above 0.1-0.2. If the CPE is low then the seeding density is increased to 5,000-50,000 cells per dish.

### EXAMPLE 5

Ultrasound Mediated Transfection of LMTK(−) Cells with Lipofectamine

LM(tk−) cells were grown at 37° C., 5% CO₂, in DMEM with 4500 mg/L D-glucose, L-glutamine, pyridoxine hydrochloride and 10% Fetal Bovine Serum. The corner wells of a 12-well dish were seeded with 200,000 cells per well (this is to ensure no interference from the ultrasound waves from other wells) 24 hours before use.

The GFP chromosomes were counted to verify approximately 1×10⁶ ACes per ml. The chromosomes were resuspended in the tube by flicking. Ten μl of chromosome suspension was removed and mixed with an equal volume of 30 mg/ml PI (propidium iodide) stain. Eight μl of the stained chromosomes was loaded onto a Petroff Hausser counting chamber and the chromosomes were counted.

The medium was removed from the cells, and the cells were washed twice with HBSS (without phenol red, Gibco BRL) warmed to 37° C. 500 μl of the warmed HBSS was added to each well of cells (1 μl) LipofectAMINE (Gibco BRL) was added to each well. The plates were then sealed with parafilm tape and shaken gently at 20 rpm at room temperature for 30 minutes (Stagger plates-10 minutes for ease of handling).

After incubation Ultrasound gel (Other-Sonic Generic Ultra sound transmission gel, Pharmaceutical Innovations, Inc., Newark, N.J.) was applied to the 2.5 cm sonoporator head. Ultrasound was applied with an ImaRX Sonoporator 100 at an output energy of 2.0 Watt/cm2, for 60 seconds, through the bottom of the plate of cells. After ultrasound of the well one chromosome per seeded cell (2×10⁵) or 200 μl GFP ACes in sheath buffer (15 nM Tris HCl, 0.1 mM EDTA, 20 mM NaCl, 1% hexylene glycol, 100 mM glycine, 20 μM spermine and 50 μM spermidine) are added immediately to the well. (Repeat until all samples on the plate requiring ultrasound have been treated). The plate was then sealed once more with parafilm tape and shaken gently (20 rpm) for 1 hour at room temperature.

After the incubation 1 ml (DMEM with 4500 mg/L D-glucose, L-glutamine and pyridoxine hydrochloride, 10% Fetal Bovine Serum, and a 1× solution of penicillin and streptomycin from a 10000 units/ml penicillin and 10000 mg/ml Streptomycin, 100× stock solution) was added to each well and the cells were incubated 18-24 hours at 37° C.

The cells in the plates were then washed with antibiotic containing medium and 2 ml of medium was placed in each well. The cells continued to be incubated at 37° C. with 5% CO₂ until 48 hours after transfection/sonoporation. The cells were then trypsinised and resuspended at a concentration of 1×10⁶ in DMEM to be analyzed by flow cytometry.

Results: Flow analysis was performed on a FACS Vantage (BDIS, San Jose, Calif.) equipped with a turbo-sort option and two inova 305 lasers (Coherent, Palo Alto, Calif.). The GFP signal excitation is at 488 nm and the emission detected in FL1 using a 500nm long pass filter. Analysis of the transfected cells generated populations of GFP positive cells ranging from 13-27%. Non-sonoporated control value was 5%.

### EXAMPLE 6

Ultrasound Mediated Transfection with Saint-2

A. Ultrasound Mediated Transfection of CHO-KI Cells with Saint-2

CHO-KI cells were grown at 37°, 5% CO₂, in CHO-S-SFM 2 Medium, (Gibco BRL, Paisley, UK). Between 2×10⁵ and 5×10⁵ cells were plated onto sterile glass slides in a 12 well plate 24 h before usage.

Transfection of the cells was performed as follows. The medium was removed from the cells, and the cells were washed twice with HBSS (Hanks balanced salt solution without Phenol Red (Gibco BRL, UK)) at 37° C. Then 500 μl HBSS at 37° C. was added per well, followed by 10 μl of the freshly prepared vesicle solution (prepared in Example 2) to yield a final concentration of 23.3 nmol/ml.

Alternatively, the medium was removed from the cells, and the cells were washed twice with HBSS. 500 μl HBSS/lipid solution at 37° C. was added to each well. The HBSS/lipid solution was prepared by adding 1 μl ethanolic lipid solution (prepared as described above) to 500 μHBSS under vigorous vortexing. The plates were then sealed with parafilm tape and shaken gently at room temperature for 30 min. After incubation, ultrasound was applied at an output energy of 0.5 Watt/cm² for 60 sec through the bottom of the plate to the cells. The ultrasound was mediated by an ultrasound gel (Aquasonic 100, Parker, N.J.) between transducer and plate. The ultrasound was applied with an ImaRx Sonoporator 100. Immediately after applying ultrasound one GFP chromosome per seeded cell (2×10⁵-5×10⁵) (prepared in Example 1) was added. The plate was then sealed again and shaken gently for 1 h at room temperature. After the incubation 1 ml medium (CHO—S—SFM 2 with 10% Fetal Calf Serum, 10000 μg/ml Penicillin and 10000 μg/ml Streptomycin Gibco BRL, Paisley, UK) was added to each well and the cells were incubated for 24 h at 37° C. The cells were then washed with medium and 1 ml medium was added and the cells were incubated at 37° for another 24 h. Detection of expressed genes was then assayed by microscopy or detection of the transferred chromosome by FISH analysis.

The negative control was performed in the same way, but with no chromosomes added to the cells.

Results

After transfection, using visual inspection, 30% of the cells remained on the glass slide of which 10% were positive for green fluorescent protein expression after 48 hours (3% of original population). After culturing for two weeks, FISH was performed on the cells and 1.4% of the cells contained an intact artificial chromosome.

B. Ultrasound Mediated Transfection of Hep-G2 Cells with Saint-2

Hep-G2 cells were grown at 37° C., 5% CO₂, in DMEM with 4500 mg/l Glucose, with Pyridoxine/HCL, 10% Fetal Calf Serum, 10000 μg/ml Streptomycin and 1000 μg/ml Penicillin. Between 2×10⁵ and 5×10⁵ cells were plated onto sterile glass slides in a 12 wells plate 24 hours before usage.

Cells were transfected with GFP chromosomes using the procedure of Example 6A except that the CHO—KI medium was replaced with Hep-G2 medium.

Results

After transfection, 30% of the cells remained on the glass slide. 80% of these cells were positive for green fluorescent protein expression.

C. Ultrasound Mediated Transfection of A9 Cells with Saint-2

A9 cells were grown at 37° C., 5% CO₂, in DMEM with 4500 mg/l Glucose, with Pyridoxine/HCL, 10% Fetal Calf Serum, 10000 μg/ml Streptomycin and 10000 μg/ml Penicillin (GIBCO BRL, Paisley, UK). Between 2×10⁵ and 5×10⁵ cells were plated onto sterile glass slides in a 12 well plate 24 h before usage.

Cells were transfected with GFP chromosomes using the procedure of Example 6A except that CHO-KI medium was replaced with A9 medium.

Results

After transfection, 30% of the cells remained on the glass of which 50% were positive for green fluorescent protein expression.

### EXAMPLE 7

Delivery of ACes into Synoviocytes, Skeletal Muscle Fibroblasts and Skin Fibroblasts

A mammalian (murine) ACes artificial chromosome (˜60 Mb) containing primarily murine pericentric heterochromatin, and including a reporter gene (lacZ) and a hygromycin B selectable marker gene, prepared as described in U.S. Pat. Nos. 6,025,155 and 6,077,697 and PCT Application Publication No. WO 97/40183 was delivered into primary rat fibroblast-like synoviocytes, rat skin fibroblasts and a rat skeletal muscle fibroblast cell line (L8 cells; ATCC Accession No. CRL-1769). Prior to delivery, the ACes were labelled with iododeoxyuridine (IdUrd) as described herein.

Preparation of Cells

Primary fibroblast-like synoviocytes and rat skin fibroblasts were obtained from rats using standard methods [see, e.g., Aupperle et al. (1999) J. Immunol. 163:427-433 and Alvaro-Garcia et al. (1 990) J. Clin. Invest. 86:1790]. Such methods include isolation of synoviocytes from rodent knees generally by removal of skin and muscle, followed by mincing of knee joint tissue. The minced tissue is then incubated with collagenase, filtered through nylon mesh and washed extensively. Cells can be cultured overnight, after which time non-adherent cells are removed. Adherent cells can be cultured and passaged by replating at a dilution when the cultures reach confluence. The cells were plated at 50,000-75,000 cells per 6-well dish in media containing low glucose DMEM, 1-glutamine, penicillin/streptomycin and 20% FBS. The cells were grown in a 5% CO₂ incubator at 37° C. for 3-5 days until approximately 80% confluence or 500,000 cells per well.

Transfection of Cells with ACes

One million IdUrd-labelled ACes were complexed with 2, 5 or 10 μl of Superfect (Qiagen) or Lipofectamine Plus (Life Technologies; Gibco) as follows. Complexing with Superfect was conducted for 10 minutes at room temperature. For complexing with Lipofectamine Plus, the indicated amounts of PLUS reagent were added to 1 million ACes and complexed at room temperature for 15 minutes. Next the indicated amounts of Lipofectamine were added into 200 μl of low glucose DMEM (no FBS) and combined with the ACes/PLUS complex for 15 minutes at room temperature. The complexed ACes were then added dropwise to the cells in 600 μl media (final volume of approximately 1.4 ml). After 3 hrs at 37° C. in a 5% CO₂ incubator, a total volume of 3 ml of culture media (low glucose DMEM, I-glutamine, penicillin/streptomycin and 20% FBS) was added. After 24-48 hrs, the cells were trypsinized to form a single cell suspension, centrifuged to remove the supernatant and then fixed in cold 70% ethanol for a minimum of one hour. An aliquot of the fixed cells was saved for microscopic analysis.

FITC-Conjugated Antibody Labelling of ACes

Following transfection, the ACes were labelled with FITC-conjugated antibody that specifically binds to BrdU- or IdUrd-labelled nucleic acids and the cells were analyzed by FACs for FITC fluorescence and microscopic staining. Fixed cells were denatured in 2N HCl and 0.5% Triton-X for 30 minutes at room temperature. After denaturation, the cells were neutralized by a series of wash steps at 4° C. To minimize background staining, the sample was resuspended in PBS and 4% FBS or BSA and 0.1% Triton-X (blocking buffer) for a minimum of 15 minutes. The cells were then pelleted and exposed for 2 hours at room temperature to FITC-conjugated antibody. After the cells were washed with blocking buffer, the sample was ready for flow cytometry analysis. Samples for microscopic analysis were dried on slides and the above staining protocol was followed, except that BrdU/IdUrd antibody was diluted ⅕ and exposed to cells for 24 hours.

Results

The delivery of intact ACes was detected within 24 to 48 hours post transfection. The number of cells recovered at 24 hours post-transfection was determined by counting an aliquot using a Coulter Counter. To determine the control plating efficiency of a recipient cell line or plating efficiency of the transfected cells, the cells were plated at 1000-10,000 cells per 10 cm petri dish in growth medium and left stationary in a 5% CO₂ incubator at 37° C. for 10 days. At that point, the number of viable colonies was determined. The normalized plating efficiency was calculated as described herein.

When Superfect was used as a delivery agent, the percent delivery into fibroblast-like synoviocytes as determined by flow cytometry ranged from ˜24% to ˜66.3%. The normalized % plating efficiency was ˜36% when 2 μl of Superfect was used and ˜16% when 5 μl of Superfect was used. Higher doses of Superfect were associated with toxicity and multiple ACes per cell as compared to lower doses. When Lipofectamine Plus was used as a delivery agent, the percent delivery into fibroblast-like synoviocytes as determined by flow cytometry ranged from ˜11% to ˜27% with percent delivery increasing with increasing doses of agent.

L8 and rat skin fibroblasts (RSF) that had been transfected with ACes were grown under hygromycin B selection and analyzed for lacZ expression. While in this example, a hygromycin selection gene was included in the ACes, there are numerous other selectable marker genes that can be used in connection with the transfer of heterologous nucleic acids into cells when it is desirable to include such genes. Such selection systems are known to those of skill in the art. A choice of selectable marker gene can, for instance, take into account the level of toxicity of the selection agent on the host cell for transfection. Identification of an appropriate selectable marker gene is routine employing the guidance provided herein.

Clones of L8 cells and RSF cells expressing lacZ were identified. These results demonstrate that IdUrd-labelled ACes can be delivered efficiently into primary cells as well as cell lines and that transgenes contained in the ACes are expressed in the transfected cells.

### EXAMPLE 8

Ex vivo Transfer of Reporter Genes into Rat Joints

To examine transfer of a heterologous gene into an in vivo environment and expression of the gene in vivo, L8 cells transfected with ACes as described above were injected into the ankle joint of rats with adjuvant-induced arthritis. On day 0, adjuvant induction of arthritis was performed on Lewis rats. Methods for adjuvant induction of arthritis in animal models are known in the art [see, e.g., Kong et al. (1999) Nature 4023:304-309]. In one exemplary protocol for adjuvant induction of arthritis, Lewis rats are immunized at the base of the tail with 1 mg Mycobacterium tuberculosis H37 RA (Difco, Detroit, Mich.) in 0.1 ml mineral oil on day 0. Paw swelling typically begins around day 10.

On day 12, intra-articular injection of transfected L8 cells (˜0.7×10⁶ cells) or untransfected control cells into the right ankle joint was performed. On day 14, the rats were sacrificed in order to analyze the joints for the presence of transplanted transfected L8 cells.

Different tissues of the sacrificed rats were examined by RT-PCR analysis for the presence of lacZ mRNA. Total mRNA was extracted from the tissues and RT-PCR was performed using primers specific for the lacZ gene. The an amplification product was detected only in the synovium, and not in the other tissues (liver, kidney, heart, spleen and lung). Synovium from the sacrificed rats was also analyzed by in situ enzymatic staining X-gal staining for β-gal activity. After snap freezing of the synovium, 8 μm and 20 μm sections were cut, counter-stained with Mayer's hematoxylin, and analyzed for blue staining of the cells. Staining was detected in synovium injected with L8 cells that had been transfected with ACes but not in synovium injected with untransfected cells. These results demonstrate successful ex vivo gene transfer in a rat adjuvant arthritis model using ACes containing a marker gene and thus the feasibility of treating arthritis and other connective tissue diseases using ACes as non-viral vectors for gene therapy.

### EXAMPLE 9

A Flow Cytometry Technique for Measuring Delivery of Artificial Chromosomes

Production cells lines (see Example 1) were grown in MEM medium (Gibco BRL) with 10% fetal calf serum (Can Sera, Rexdale ON) with 0.168 μg/ml hygromycin B (Calbiochem, San Diego, Calif.). Iododeoxyuridine or Bromodeoxyuridine was added directly to culture medium of the production cell line (CHO E42019) in the exponential phase of growth. Stock Iododeoxyuridine was made in tris base pH 10. Bromodeoxyuridine stocks in PBS. Final concentrations of 0.05-1 μM for continuous label of 20-24 hours of 5-50 μM with 15 minute pulse. After 24 hours, exponentially growing cells were blocked in mitosis with colchicine (1.0 μg/ml for 7 hours before harvest. Chromosomes were then isolated and stained with Hoechst 33258 (2.5 μg/ml) and chromomycin A3 (50 μg/ml). Purification of artificial chromosomes was performed using a FACS Vantage flow cytometer (Becton Dickinson Immunocytometry systems, San Jose, Calif.). Chromomycin A3 was excited with the primary laser set at 457 nm, with emission detected using 475 nm long pass filter. Hoechst was excited by the secondary UV laser and emission detected using a 420/44 nm band-pass filter. Both lasers had an output of 150 mW. Bivariate distribution showing cell karyotype was accumulated from each sort. ACes were gated from other chromosomes and sorted. Condensing agents (hexylene glycol, spermine, and spermidine) were added to the sheath buffer to maintain condensed intact chromosome after sorting. IdU labeling index of sorted chromosomes was determined microscopically. Aliquot (2-10 μl) of sorted chromosomes was fixed in 0.2% formaldehyde solution for 5 minutes before being dried on clean microscopic slide. Microscope sample was fixed with 70% ethanol. Air-dried slide was denatured in coplin jar with 2N HCl for 30 minutes at room temperature and washed 2-3 times with PBS. Non specific binding was blocked with PBS and 4% BSA or serum for minimum of 10 minutes. A ⅕ dilution of FITC conjugated IdU/BrdU antibody (Becton Dickinson) with a final volume of 60-100 μl was applied to slide. Plastic strips, Durra seal (Diversified Biotech, Boston, Mass.) were overlaid on slides, and slides were kept in dark at 4% C in humidified covered box for 8-24 hours. DAPI (Sigma) 1 μg/ml in Vectorshield was used as counterstain. Fluorescence was detected using Zeiss axioplan 2 microscope equipped for epiflorescence. Minimum of 100 chromosomes was scored for determining % labeled. Unlabeled chromosomes were used as negative control.

The day before the transfection, trypsinize V79-4 (Chinese Hamster Lung fibroblast) cells and plate at 250,000 into a 6 well petri dish in 4 mls DMEM (Dulbecco's Modified Eagle Medium, Life Technologies) and 10% FBS (Can Sera Rexdale ON). The protocol was modified for use with LM (tk−) cell line by plating 500,000 cells. Lipid or dendrimer reagent was added to 1×10⁶ ACes sorted in ˜800 μl sort buffer. Exemplary protocol variations are set forth in Table 1. Chromosome and transfection agents were mixed gently. Complexes added to cells drop-wise and plate swirled to mix. Plates were kept at 37° C. in a 5% CO₂ incubator for specified transfection time. The volume in a well was then made up to 4-5 ml with DMEM and 10% FBS. Recipient cells left for 24 hours at 37° C. in a 5% CO₂ incubator. Trypsinize transfected cells. Samples to be analyzed for IdU labeled chromosome delivery are fixed in cold 70% ethanol and stored at −20° C., to be ready for IdU antibody staining. Samples to be grown for colony selection are counted and then transferred to 10-cm dishes at densities of 10,000 and 100,000 cells in duplicate with remaining cells put in a 1 5 cm dish. After 24 hours, selection medium containing of DMEM and 10% FBS with 0.7 mg/ml hygromycin B, #400051 (Calbiochem San Diego, Calif.) is added. Selection medium is changed every 2-3 days. This concentration of hygromycin B kills the wild type cells after selection for 7 days. At 10-14 days colonies were expanded and then screened by FISH for intact chromosome transfer and assayed for beta galactosidase expression.

TABLE 1
Delivery Transfection Protocols

| | | | | | Medium (ml) | |
|---|---|---|---|---|---|---|
| | | | Complexing | | added to wells | |
| | | Pre treatment | time | Added to | before | Transfection |
| Agent | Dilution Stock | of ACes | (minutes) | complexes | complexes | time (hours) |
| CLONFECTIN | 2-8 μg in | | 20 | 1.8 ml of | | 4 |
| | NaCl-HEPES | | | serum free | | |
| CYTOFECTENE | | | 10-20 | 200 μl of 50% | | 24 |
| | | | | FBS plus | | |
| | | | | DMEM | | |
| ENHANCER + | | Enhancer 5 | 10 | | 1.2 | 3 |
| EFFECTENE | | minutes | | | | |
| (1:5 ratio) | | | | | | |
| EU-FECTIN-1 to | | | 5-10 | | | 6 |
| 11 | | | | | | |
| FUGENE 6 | 0.5-6 μl to | | 15-45 | | | 4 |
| | final volume | | | | | |
| | of 100 μl in | | | | | |
| | serum free | | | | | |
| | medium | | | | | |
| GENEPORTER 2 | 2.5 μl added | | 2-10 | | | 2-4 |
| | to 150 μl of | | | | | |
| | serum free | | | | | |
| | medium | | | | | |
| LIPOFECTAMINE | | | 15 | | | 3 |
| LIPOFECTAMINE | | | 20 | | 2.5 | 5 |
| 2000 | | | | | | |
| METAFECTENE | diluted into | | 15-45 | | 0.8 | 6 |
| | 60 μl serum | | | | | |
| | free medium | | | | | |
| PLUS + | | PLUS and 200 | 15 | | | 3 |
| LIPOFECTAMINE | | μl of DMEM | | | | |
| (1:1 and 3:2 | | for 15 minutes | | | | |
| ratio) | | | | | | |
| SUPERFECT | | | 10 | | 0.6 | 3 |

IDU Antibody Labeling

A standard BrdU staining flow cytometry protocol (Gratzer et al. Cytometry (1981);6:385-393) was used except with some modifications at the neutralization step, the presence of detergent during denaturation and the composition of blocking buffer. Between each step samples are centrifuged at 300 g for 7-10 minutes and supernatant removed. Samples of 1-2 million cells are fixed in 70% cold ethanol. Cells are then denatured in 1-2 ml of 2N HCL plus 0.5% triton X for 30 minutes at room temperature. Sample undergoes 3-4 washes with cold DMEM until indictor is neutral. Final wash with cold DMEM plus 5% FBS. Blocking/permeabilization buffer containing PBS, 0.1% triton X and 4% FBS is added for 10-15 minutes before pelleting sample by centrifugation. Add 20 μl of IdU/BrdU FITC conjugated B44 clone antibody (Becton Dickinson Immunocytometry Systems, San Jose, Calif.) to pellet and leave for 2 hours at room temperature in the dark with agitation every 30 minutes. Wash cells with block/permeabilization buffer and resuspend in PBS for flow analysis.

Flow Cytometry Detection of Fluorescent IDUrdD Labeled ACes

Percentage of transfected cells containing IdU labeled ACes was determined using a flow cytometry with an argon laser turned to 488 nm at 400 mW. FITC fluorescence was collected through a standard FITC 530/30-nm band pass filter. Cell populations were gated on the basis of side scatter versus forward scatter to exclude debris and doublets. Data was accumulated (15,000 events) to form bivariate channel distribution showing forward scatter versus green fluorescence (IdU-FITC). The fluorescence level at which cells were determined to be positive was established by visual inspection of the histogram of negative control cells, such that approximately 1% appeared in the positive region.

Results:

The transfection delivery results of IdU labeled ACes are set forth in Table 2.

TABLE 2

| | DOSE | DELIVERY |
|---|---|---|
| | Microliters agent | % IdU positive |
| COMPOUND | added per 1 | (24 hours) |
| NEGATIVE CONTROL (ACes | 0 | 0.12 |
| only) | | |
| CLONFECTIN | 6 | 0.61 |
| CYTOFECTENE | 8 | 14.67 |
| ENHANCER + EFFECTENE | 1.6, 10 | 17.08 |
| (1:5) | | |
| EU-FECTIN-1 | 10 | 4.57 |
| EU-FECTIN-2 | 5 | 0.14 |
| EU-FECTIN-3 | 10 | 0.69 |
| EU-FECTIN-4 | 10 | 0.24 |
| EU-FECTIN-5 | 10 | 0.41 |
| EU-FECTIN-6 | 10 | 0.46 |
| EU-FECTIN-7 | 10 | 1.21 |
| EU-FECTIN-8 | 10 | 1.58 |
| EU-FECTIN-9 | 10 | 0.6 |
| EU-FECTIN-10 | 10 | 0.77 |
| EU-FECTIN-11 | 5 | 1 |
| FUGENE | 8 | 0.49 |
| GENEPORTER | 5 | 22.12 |
| LIPOFECTAMINE | 25 | 17.81 |
| LIPOFECTAMINE 2000 | 30 | 10.96 |
| PLUS + LIPOFECTAMINE | 12, 12 | 12.2 |
| (1:1) | | |
| PLUS + LIPOFECTAMINE | 24, 16 | 26.97 |
| (3:2) | | |
| METAFECTENE | 10 | 14.14 |
| SUPERFECT | 2 | 27.67 |

### EXAMPLE 10

Delivery of ACes into LM(tk−) and Hff Cells by Cell Synchronization

A mammalian (murine) derived 60 Mb ACes artificial chromosome was used for all transfections. The ACes artificial chromosome primarily contains mouse major satellite DNA, interspersed with blocks of ‘payload’ genetic material, including a reporter gene (lacZ) and a hygromycin B selectable marker gene. The ACes were prepared and purified as previously described herein and delivered into synchronized, immortalized murine LM(tk−) and primary human foreskin fibroblast (Hff) cells. Two common cell cycle arrest agents, thymidine and nocodazole, were chosen for cell synchronization. Thymidine incorporation accumulates cells in G₀-G₁ phase, and nocadozole acts by arresting cells in G₂-M. Removal of the arresting agents allows the cells to continue through the cell cycle in a synchronous manner for approximately 24 hours. Two cationic transfection agents were used to deliver the ACE systems into cells, Superfect (Qiagen, Inc., Mississauga, Ontario) and LipofectAMINE PLUS (Gibco BRL). Transfection with these agents was carried out according to manufacturer's protocols.

Nocodazole Cell Synchronization

Optimization of Nocodozole Cell Synchronization in LM(tk−) Cells

To determine the optimum blocking concentration of nocodazole, nocodazole concentrations were titrated against LM(tk−) cells to generate a dose response. Timing of the optimal blocking concentration (100 ng/ml) was then assessed over 0-1 6 hours using flow cytometry. To determine the cell cycle kinetics of LM(tk−) cell populations at different time points, cells were stained with propidium iodide (PI) at a concentration of 20 pg/ml for 1×10⁶ cells and analyzed with a FACS Vantage SE (Becton Dickinson Immunocytometry Systems, San Jose, Calif.) equiped with an argon laser tuned to 488 nm at 200 mW. Whole intact cells were gated on forward scatter (FSC, representing cell size) versus side scatter (SSC, representing internal granularity) and PI emission was detected with a 630/22 filter. A list mode file was generated with a minimum of 20,000 whole intact cells analyzed. The proportion of cells in G₀-G₁, S and G₂-M were documented for each time point. Optimum cell-cycle arrest at the G₂/M boundary was achieved by incubating cells with 100 ng/ml nocodazole in serum containing DMEM medium for 6 hours. After 6 hours of nocodazole exposure at 100 ng/ml, approximately 20% of cells were in S phase and 50% of cells were at G₂/M. Cell cycle kinetics were again monitored by flow cytometry upon release of the nocodazole block and at frequent intervals over a 16 hour period.

Optimizing the Time Point for Transfection in LM(tk−) Cells

Cells were plated at 7.5×10⁵ cells per well of a 6-well plate 24 hours prior to nocodazole treatment. The cells were incubated with nocodazole for 6 hours. After the 6 hour incubation, wells were hosed with medium to dislodge the suspension fraction of cells. Nocodazole containing medium was removed and the wells were washed with fresh medium. The wash medium was aspirated and fresh medium was replaced in the wells. To determine the optimum time point for transfection, different cell samples were transfected at different time points after release of the nocodazole block. The time points were 0, 2, 4, 6, 8, 12, and 16 hours after release of the nocodazole block. Following transfer, the cells were plated and transfectants identified using hygromycin selection. Highest colony formations were achieved when transfection occurred 2-4 hours post nocodazole block. At these time points, cell population distribution included approximately 10% of cells in G₀/G₁, 20% in S, and 70% in G₂/M. Transfectants were analyzed using FISH to determine the quality of the transferred material. Randomly selected drug resistant colonies were analyzed and the number of colonies where at least 50% of the cells had an intact ACes were scored. Selected colonies derived from nocodazole synchronized transfections had a larger percentage of cells that maintained intact ACes compared with asynchronous control colonies. With both LipofectAMINE PLUS or Superfect, the percentage of colonies meeting this criteria was 4% (2/49) for asynchronous control colonies and 43% (20/47) for nocodazole synchronized colonies. β-galactosidase staining was used to confirm expression of the reporter gene.

Delivery of ACes into LM(tk−) Cells by Cell Synchronization.

Cells were synchronized by nocodazole treatment (100 ng/ml final concentration) for 6 hours prior to transfection. The mitotic cells are gently removed by rinsing the plate with growth medium. Adherent cells were transfected at 2-hour intervals (approximately 1 million cells per interval) with Superfect (10 ul) over an 8-hour time course. Twenty-four hours post-transfection the cells are counted (Coulter counter) and a small fraction are plated for a modified toxicity assay. The remaining cells were plated separately in growth medium and 24 hours later were placed in selection medium (growth medium with 1 mg/ml hygromycin B). The selection medium was changed every 2-3 days until colonies formed. LipofectAMINE PLUS concentrations used were 12 ul for both the LipofectAMINE and PLUS reagent. Transfection conditions were otherwise identical and selection of LM(tk−) cells was acomplished as described above.

Delivery of ACes into Hff Cells by Cell Synchronization.

The protocol for Hff cells differs from that used for LM(tk−) cells in the following details: nocodazole concentration was 45 ng/ml for an 8 hour exposure; 4 ul Superfect was used; Transfection were carried out an half hour intervals over a two hour time frame; colony selection was not done as the Hff cells are hygromycin B sensitive. Cells were transfected with either 4 ul Superfect or 10 ul LipofectAMINE PLUS (10 ul for each component LipofectAMINE and PLUS; Invitrogen). Transfections were carried out every half hour over a 2-hour timeframe (cells returned to logarithmically growing state after this short time). The Hff cells are sensitive to hygromycin B therefore transfections were carried out using the RFP ACes and expression was monitored by flow cytometry.

Thymidine Cell Synchronization

Optimization of Thymidine Cell Synchronization in LM(tk−) Cells

To determine the optimum parameters for cell cycle synchronization, thymidine was administered to cell samples in a dose range of 1-20 mM. Samples were analyzed by flow cytometry to determine the cell cycle kinetics of LM(tk−) cell populations at frequent intervals over a 16 hour time period. Optimum synchronization of cells in G₀/G₁ was achieved by incubating LM(tk−) cells with 10 mM thymidine in serum containing DMEM medium for 16 hours. The thymidine block was released by washing the cells twice in serum-free medium and replacing the medium with complete medium containing 24 μM 2′-deoxycytidine 5′ mono-phosophate (dCMP; Sigma D-7750, St. Louis, Mo.). Cell cycle kinetics were again monitored by flow cytometry upon release of the thymidine block and at frequent intervals over a 16 hour period.

Optimizing the Time Point for Transfection in LM(tk−) Cells

Cells were plated at 7.5×10⁵ cells per well of a 6-well plate 24 hours prior to thymidine treatment. The cells were incubated with medium containing 10 mM thymidine for 16 hours. After the 16 hour incubation, the thymidine block was released by washing the cells twice in serum-free medium and replacing the medium with 24 μM deoxycytidine 5′ mono-phosophate (dCMP) supplemented complete medium. To determine the optimum time point for transfection, different cell samples were transfected at different time points after release of the thymidine block. The time points were 0, 2, 4, and 6 hours after release of the thymidine block. Following transfer, the cells were plated and transfectants identified using hygromycin selection. Transfectants were analyzed using FISH to determine ACE system integrity and β-galactosidase staining to confirm expression of the reporter gene. Optimal transfection was achieved at 4-5 hours post thymidine release.

Hff Cells

A protocol similar to that used with the LM(tk−) cells was employed with the exception of an alteration in the dose of thymidine (5 mM).

Results

Studies indicate successful achievement of reversible arrest of the LM(tk−) and Hff cells with both nocodazole and thymidine. Studies further show that synchronization and release of cells at specific points in the cell cycle increases the efficiency of ACes transfection into LM(tk−) and Hff cells. Using nocodazole or thymidine with either of two transfection agents (Superfect of LipofectAMINE PLUS), transfection efficiencies were enhanced 2.5-6 fold above control transfections on asynchronous cell populations. Transfection efficiency was strongly correlated with the fraction of cells in G₂/M (R2=0.827), regardless of which blocking agent was used suggesting that optimal transfection is achieved when the majority of the cells are in the G₂/M phase of the cell cycle, regardless of where the cell population is originally arrested. Qualitative assessments of the chromosomes also revealed that delivery of ACes to cells during the optimized transfection point led to higher proportions of intact artificial chromosomes compared to asynchronous controls.

Since modifications will be apparent to those of skill in this art, it is intended that this invention be limited only by the scope of the appended claims.

1. A method for introducing a large nucleic acid molecule into a cell, comprising:obtaining a cell that is in a pre-selected phase; and
contacting the cell with the large nucleic acid molecule, whereby the large nucleic acid molecule is delivered into the cell.

2. The method of claim 1, wherein the nucleic acid molecule is greater than about 0.6 megabase.

3. The method of claim 1, wherein the nucleic acid molecule is greater than about 1 megabase.

4. The method of claim 1, wherein the nucleic acid molecule is greater than about 5 megabases.

5. The method of claim 1, wherein the nucleic acid molecule is a natural chromosome, an artificial chromosome, or a fragment of a chromosome that is greater than about 0.6 megabase or naked DNA that is greater than about 0.6 megabase.

6. The method of claim 1, wherein the nucleic acid molecule is an artificial chromosome.

7. The method of claim 1, wherein the nucleic acid molecule is an artificial chromosome expression system (ACes).

8. The method of claim 1, wherein the nucleic acid molecule and/or the cell is exposed to a delivery agent.

9. The method of claim 8, wherein the delivery agent comprises a cationic compound.

10. The method of claim 9, wherein the cationic compound is selected from the group consisting of a cationic lipid, a cationic polymer, a mixture of cationic lipids, a mixture of cationic polymers, a mixture of a cationic lipid and a cationic polymer, a mixture of a cationic lipid and a neutral lipid, polycationic lipids, non-liposomal forming lipids, activated dendrimers, and a pyridinium chloride surfactant.

11. The method of claim 8, wherein the delivery agent is a composition that comprises one or more cationic compounds, wherein the compound is selected from the group consisting of 2,3-dioleyloxy-N-[2(spermine-carboxamido)ethyl]-N,N-dimethyl-1-propanaminiumtrifluoro-acetate (DOSPA), C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O4S₂.8CF₃CO₂H, C₄₁H₇₈NO₈P) C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, and (1-methyl-4-(1-octadec-9-enyl-nonadec-10-enylenyl) pyridinium chloride.

12. The method of claim 1, wherein:
the nucleic acid molecule is exposed to an agent that increases contact between the nucleic acid molecule and the cell; and
the cell is exposed to an agent that enhances permeability of the cell.

13. The method of claim 1 2, wherein the exposure of the cell to an agent that enhances permeability comprises applying ultrasound or electrical energy to the cell.

14. The method of claim 1, wherein the cell is selected from the group consisting of a nuclear transfer donor cell, a stem cell, a primary cell, a cell from an immortalized cell line, an embyronic cell, a tumor cell, a transformed cell and a cell capable of the generation of a specific organ.

15. The method of claim 1, wherein the cell is selected from the group consisting of a primary cell, an immortalized cell, an embryonic cell, a stem cell, a transformed cell and a tumor cell.

16. The method of claim 1, wherein the cell is selected from the group consisting of a nuclear transfer donor cell, a stem cell, and a cell capable of the generation of a specific organ.

17. The method of claim 1, wherein the cell is a mammalian cell.

18. The method of claim 1, wherein the cell is a rodent cell or a human cell.

19. The method of claim 1, wherein the cell is a fibroblast.

20. The method of claim 1, wherein the cell is a synoviocyte.

21. The method of claim 20, wherein the cell is a fibroblast-like synoviocyte.

22. The method of claim 1, wherein the pre-selected phase of the cell is determined by a method comprising:
introducing a large nucleic acid into cells at different phases;
determining and comparing the efficiency of delivery and/or of transfection of the nucleic acid into the cells at different phases; and
selecting a phase at which the efficiency of delivery and/or of transfection is increased relative to efficiency of delivery and/or transfection at other phases.

23. The method of claim 1, wherein the cell that is in a pre-selected phase is obtained by a method comprising:
exposing one or more cells to a cell cycle arrest agent;
exposing the one or more cells to conditions that permit cell cycling;
selecting a cell that is in the pre-selected phase.

24. The method of claim 23, wherein the arrest agent is an anti-microtubule agent.

25. The method of claim 23, wherein the arrest agent is nocodazole or thymidine.

26. The method of claim 1, wherein the pre-selected phase is G2/M.

27. The method of claim 23, wherein exposure of the one or more cells to an arrest agent results in arrest of the one or more cells in mitosis or G0/G1.

28. The method of claim 1, wherein the nuclear membrane of the cell is absent.

29. The method of claim 1, wherein a plurality of cells in a pre-selected phase is obtained.

30. The method of claim 29, wherein the plurality of cells is in a synchronous population of cells.

31. The method of claim 30, wherein the efficiency of transfection of the large nucleic acid molecule to cells in a synchronous population of cells is selected from among at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5 and at least 10-fold greater than the efficiency of transfection of the large nucleic acid molecule to cells in an asynchronous population of cells.

32. The method of claim 30, wherein the large nucleic acid molecule is a chromosome or artificial chromosome and the percentage of cells comprising an intact delivered chromosome after contact of a synchronous population of cells with the chromosome or artificial chromosome is greater than the percentage of cells comprising an intact delivered chromosome after contact of an asynchronous population of cells with the chromosome or artificial chromosome.

33. The method of claim 32 wherein the artificial chromosome is an ACes.

34. A method for introducing a chromosome or functional fragment thereof into a cell, comprising contacting the cell with a chromosome or fragment thereof that is in the same or similar phase as the cell, whereby the chromosome or functional fragment thereof is delivered into the cell.

35. The method of claim 34 wherein the chromosome is an artificial chromosome.

36. The method of claim 35, wherein the chromosome is an ACes.

37. A method for ex vivo gene therapy, comprising:
obtaining a cell that is in a pre-selected phase;
contacting the cell with a nucleic acid molecule, whereby the nucleic acid molecule is delivered into the cell; and introducing the cell into a subject.

38. The method of claim 37, wherein the nucleic acid molecule is a large nucleic acid molecule.

39. The method of claim 37, wherein the nucleic acid molecule is greater than about 0.6 megabase.

40. The method of claim 37, wherein the nucleic acid molecule is greater than about 1 megabase.

41. The method of claim 37, wherein the nucleic acid molecule is greater than about 5 megabases.

42. The method of claim 37, wherein the nucleic acid molecule is a natural chromosome, an artificial chromosome, or a fragment of a chromosome that is greater than about 0.6 megabase or naked DNA that is greater than about 0.6 megabase.

43. The method of claim 37, wherein the nucleic acid molecule is an artificial chromosome.

44. The method of claim 37, wherein the nucleic acid molecule is an artificial chromosome expression system (ACes).

45. The method of claim 37, wherein the nucleic acid molecule and/or the cell is exposed to a delivery agent.

46. The method of claim 45, wherein the delivery agent comprises a cationic compound.

47. The method of claim 46, wherein the cationic compound is selected from the group consisting of a cationic lipid, a cationic polymer, a mixture of cationic lipids, a mixture of cationic polymers, a mixture of a cationic lipid and a cationic polymer, a mixture of a cationic lipid and a neutral lipid, polycationic lipids, non-liposomal forming lipids, activated dendrimers, and a pyridinium chloride surfactant.

48. The method of claim 45, wherein the delivery agent is a composition that comprises one or more cationic compounds, wherein the compound is selected from the group consisting of 2,3-dioleyloxy-N-[2(spermine-carboxamido)ethyl]-N,N-dimethyl-1-propanaminiumtrifluoroacetate (DOSPA), C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O₄S₂.8CF₃CO₂H, C₄₁H₇₈NO₈P) C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, and (1-methyl-4-(1-octadec-9-enyl-nonadec-10-enylenyl) pyridinium chloride.

49. The method of claim 37, wherein:
the nucleic acid molecule is exposed to an agent that increases contact between the nucleic acid molecule and the cell; and
the cell is exposed to an agent that enhances permeability of the cell.

50. The method of claim 49, wherein the exposure of the cell to an agent that enhances permeability comprises applying ultrasound or electrical energy to the cell.

51. The method of claim 37, wherein the cell is a mammalian cell.

52. The method of claim 37, wherein the cell is a human cell.

53. The method of claim 37, wherein the cell is a synoviocyte.

54. The method of claim 37, wherein the cell is a fibroblast-like synoviocyte.

55. The method of claim 37, wherein the pre-selected phase is determined by a method comprising:
introducing nucleic acid into cells at different phases;
determining and comparing the efficiency of delivery and/or transfection of the nucleic acid into the cells at different phases; and
selecting a phase at which the efficiency of delivery and/or transfection is increased relative to efficiency of delivery and/or transfection at other phases.

56. The method of claim 37, wherein the cell that is in a pre-selected phase is obtained by a method comprising:
exposing one or more cells to a cell cycle arrest agent;
exposing the one or more cells to conditions that permit cell cycling;
selecting a cell that is in the pre-selected phase.

57. The method of claim 56, wherein the arrest agent is an anti-microtubule compound.

58. The method of claim 56, wherein the arrest agent is nocodazole or thymidine.

59. The method of claim 56, wherein the pre-selected phase cycle is G2/M.

60. The method of claim 56, wherein exposure of the one or more cells to an arrest agent results in arrest of the one or more cells in mitosis or G0/G1.

61. The method of claim 37, wherein the nuclear membrane of the cell is absent.

62. The method of claim 37, wherein a plurality of cells in a pre-selected phase is obtained and the plurality of cells is contacted with one or more nucleic acid molecules, whereby the nucleic acid molecule(s) is delivered into one or more cells, and one or more cells are introduced into the subject.

63. The method of claim 62, wherein the plurality of cells is in a synchronous population of cells.

64. The method of claim 63, wherein the efficiency of transfection of the nucleic acid molecule to cells in a synchronous population of cells is selected from among at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5 and at least 10-fold greater than the efficiency of delivery of the nucleic acid molecule to cells in an asynchronous population of cells.

65. The method of claim 63, wherein the nucleic acid molecule is a chromosome or artificial chromosome and the percentage of cells comprising an intact delivered chromosome after contact of a synchronous population of cells with the chromosome or artificial chromosome is greater than the percentage of cells comprising an intact delivered chromosome after contact of an asynchronous population of cells with the chromosome or artificial chromosome.

66. The method of claim 65 wherein the artificial chromosome is an ACes.

67. A method for ex vivo gene therapy, comprising:
contacting a cell with a chromosome or fragment thereof that is in the same or similar phase as the cell, whereby the chromosome or functional fragment thereof is delivered into the cell; and
introducing the cell into a subject.

68. The method of claim 67 wherein the chromosome is an artificial chromosome.

69. The method of claim 68, wherein the chromosome is an ACes.

70. A kit for delivering large nucleic acids into cells, comprising:
a delivery agent;
a cell cycle arrest agent; and
optionally instructions for delivering large nucleic acids into cells.

71. A method for selecting a host cell for receiving a large nucleic acid molecule, comprising:
introducing a large nucleic acid molecule into cells at different phases;
determining and comparing the efficiency of delivery and/or transfection of the nucleic acid into the cells at different phases; and
selecting a cell that is in a phase that provides for increased efficiency of delivery and/or transfection relative to the efficiency of delivery and/or transfection at other phases.

72. The method of claim 71, wherein the large nucleic acid molecule is labeled.

73. The method of claim 72, wherein the labelled cells are detected by flow cytometry, fluorimetry, cell imaging or fluorescence spectroscopy.

74. The method of claim 73, wherein the labelled cells are detected by flow cytometry.

75. The method of claim 73, wherein the label is iododeoxyuridine (IdU or IdUrd) or bromodeoxyuridine (BrdU).

76. The method of claim 73, wherein the large nucleic acid molecule is a chromosome or functional fragment thereof.

77. The method of claim 73, wherein the large nucleic acid molecule is an artificial chromosome.

78. The method of claim 77, wherein the artificial chromosome is an ACes.

79. The method of claim 1, wherein the pre-selected phase of the cell is selected from the group consisting of G1, S, G2, G2/M, M and any of the stages of the M phase.

80. The method of claim 1, wherein the pre-selected phase of the cell cycle is not G0.

81. The method of claim 1, wherein the large nucleic acid is associated with one or more proteins.

82. The method of claim 34, wherein the chromosome or functional fragment thereof is associated with one or more proteins.

83. The method of claim 23, wherein the pre-selected phase is G2/M.

84. The method of claim 37, wherein the pre-selected phase cycle is G2/M.
